(19) United States
(12) Patent Application Publication
(10) Pub. No.: US 2025/0368623 A1
(43) Pub. Date: Dec. 4, 2025

(54) PYRIDAZINONE-DERIVED COMPOUNDS FOR THE MODULATION OF MYC AND FOR MEDICAL USES

(71) Applicant: GENETIC INTELLIGENCE, INC, New York, NY (US)

(72) Inventors: Bertrand ADANVE, New York, NY (US); Yao Zong NG, Singapore (SG); Jonathan LAI, Smithtown, NY (US)

(21) Appl. No.: 18/875,028

(22) PCT Filed: Jun. 15, 2023
(86) PCT No.: PCT/US2023/068478

§ 371 Date: Dec. 13, 2024

Related U.S. Application Data
(60) Provisional application No. 63/352,514, filed on Jun. 15, 2022.

(51) Int. Cl. C07D 403/06; A61K 31/501; A61P 35/04; C07D 401/06

(52) U.S. Cl. CPC C07D 403/06; A61K 31/501; A61P 35/04; C07D 401/06; C07B 2200/05

(57) ABSTRACT

Compositions, systems, and methods are described herein for the modulation, and in particular the reduction or inhibition, of expression or activity of MYC in a cell, animal or human subject. Such compositions, systems, and methods are useful to prevent, ameliorate, or treat diseases, including cell proliferation diseases and disorders such as cancer, particularly MYC-driven cancer. Compositions are described comprising a compound of formula (I), as well as pharmaceutical compositions or medicaments thereof. Also described are methods of use of such compositions to treat, prevent, or ameliorate diseases, including cell proliferation diseases and disorders such as cancer, in particular MYC-driven cancer. Compositions comprising conjugates and complexes of a compound of formula (I) are also described, which are also useful in the methods described herein. Methods are described comprising the use of a compound of formula (I), or pharmaceutical compositions thereof, for the reduction or inhibition of MYC expression or activity, and/or for achieving one or more desirable phenotypic outcomes such as, for example, decrease in cancer cell growth or proliferation, decrease in cancer cell viability, decrease in tumor volume (i.e., tumor regression), decrease in cancer metastasis, increase in animal or human survival, or other desired outcome with respect to particular phenotypes (e.g., body weight, metabolism, etc.). Related medicaments, kits, and methods of delivery of such compositions are described. Methods are also described for the development, manufacture, and/or synthesis of a compound of formula (I), as well as pharmaceutical compositions thereof. Furthermore, methods for diagnostics and testing comprising detecting MYC expression or activity levels, as well as compositions comprising kits for diagnostics and testing, are described.

### CROSS-REFERENCE TO RELATED APPLICATIONS

This application claims priority to U.S. Provisional Application 63/352,514 (filed on Jun. 15, 2022), the disclosure of which is incorporated by reference in its entirety.

### SEQUENCE LISTING

The instant application contains a Sequence Listing, which has been filed electronically in ASCII format and is hereby incorporated by reference in its entirety. Said ASCII copy, created Jun. 9, 2023, is named 20230609-MYC.xml and is 23,426 bytes in size.

### FIELD OF THE INVENTION

This invention relates to compositions, systems, and methods for modulating, in particular reducing or inhibiting, the expression and/or activity of MYC in a cell, an animal or human subject. Such compositions, systems, and methods are useful to treat, prevent, or ameliorate diseases including cell proliferation diseases and disorders such as cancer, particularly MYC-driven cancer.

### BACKGROUND

In the following discussion certain articles and processes will be described for background and introductory purposes. Nothing contained herein is to be construed as an “admission” of prior art. Applicant expressly reserves the right to demonstrate, where appropriate, that the articles and processes referenced herein do not constitute prior art under the applicable statutory provisions.

The MYC family of proto-oncogenes consists of C-MYC (otherwise known as MYC, MYCC, V-Myc myelocytomatosis viral oncogene homolog, BHLHe39, or MRTL), N-MYC (otherwise known as MYCN, BHLHe37, V-Myc myelocytomatosis viral oncogene neuroblastoma derived homolog, MYCNOT, MODED or ODED) and L-MYC (otherwise known as MYCL, LMYC, BHLHe38, MYCL1 or V-Myc myelocytomatosis viral oncogene lung carcinoma derived homolog). In some embodiments, MYC refers to the MYC family of proto-oncogenes. In a specific embodiment, MYC refers to C-MYC. In another specific embodiment, MYC refers to N-MYC. In some embodiments, MYC refers to a polymorph, isoform, homolog, pseudogene, or mutant form of MYC. As used herein, MYC can refer to genes, RNA transcripts or protein products obtained from the expression of nucleic acids encoding MYC, unless specified or indicated otherwise.

The MYC family of genes encodes for transcription factors that play important roles in regulating cell proliferation, cell cycle, cell growth, differentiation, angiogenesis, apoptosis, immunity, stress response and oncogenesis etc. Some examples of the roles of MYC are described in Ahmadi et al. (Ahmadi et al., Journal of Hematology and Oncology, 14, 121 (2021)), Shrestha et al. (Shrestha et al., Front. Oncol., 11, Article 694320 (2021)), Eilers et al. (Eilers et al., Genes Dev., 22(20): 2755-2766 (2008)), Holzel et al. (Holzel et al., FMBO Reports, 21: 1125-1132 (2001)), Greasley et al. (Greasley et al., Nucleic Acids Res., 28: 446-453 (2000)), Trumpp et al. (Trumpp et al., Nature, 414: 768-773 (2001)), Bouchard et al. (Bouchard et al., Genes Devel., 15: 2042-2047 (2001)), Menssen et al. (Menssen et al., Proc. Nat. Acad. Sci. USA, 59: 6274-6279 (2002)), and Nesbit et al. (Nesbit et al., Blood, 92: 1003-1010 (1998)), the disclosures of which, along with their references, are incorporated herein in their entirety.

The MYC family contributes to the pathogenesis of almost all cancers. Gain-of-function of MYC is commonly observed in cancers, which can be a result of mutations, chromosomal rearrangements, gene amplification or increased expression etc. Some examples of various mechanisms and pathways by which MYC can contribute to the pathogenesis of cancers are described in Dhanasekaran et al. (Dhanasekaran et al., Nature Reviews Clinical Oncology, 19: 23-36 (2022)), Gabay et al. (Gabay et al., Cold Spring Harb. Perspect. Med., 4(6): a014241 (2014)), Dang et al. (Dang et al., Cell, 149(1): 22-35 (2012)), He et al. (He et al., Science, 281: 1509-1512 (1998)), and Rochlitz et al. (Rochlitz et al., Oncology, 53: 448-454 (1996)), the disclosures of which, along with their references, are incorporated herein in their entirety.

The MYC family is known to be drivers of multiple different cancer types. C-MYC is known to drive cancers such as, for example, breast cancer, Burkitt's lymphoma, cervical cancer, colorectal cancer (e.g., colon adenocarcinoma, rectum adenocarcinoma), esophageal carcinoma, gastric cancer (e.g., stomach adenocarcinoma), glioblastoma (e.g., glioblastoma multiforme), head and neck squamous cell carcinoma, leukemia (e.g., myeloid leukemia), liver cancer, lung cancer (e.g., non-small cell lung cancer, small cell lung carcinoma, lung squamous cell carcinoma), non-Burkitt's lymphoma, medulloblastoma, melanoma (e.g., skin cutaneous melanoma, uveal melanoma), mesothelioma, multiple myeloma, neuroblastoma, osteosarcoma, ovarian cancer, pancreatic cancer, prostate cancer, renal cancer (e.g., clear cell renal cell carcinoma), and rhabdomyosarcoma, etc. N-MYC is known to drive cancers such as, for example, astrocytoma, brain lower grade glioma, breast cancer, glioblastoma, lung cancer (e.g., small cell lung cancer), medullary thyroid carcinoma, medulloblastoma, neuroblastoma, ovarian cancer, pancreatic cancer, pheochromocytoma and paraganglioma, prostate cancer, retinoblastoma, rhabdomyosarcoma (e.g., alveolar rhabdomyosarcoma), and testicular cancer, etc. L-MYC is known to drive cancers such as, for example, small cell lung cancer, etc. The different types of cancers that are driven by C-MYC, N-MYC and L-MYC are described by Schaub et al. (Schaub et al., Cell Systems, 6(3): 282-300 (2018)), Nesbit et al. (Nesbit et al., Oncogene, 18: 3004-3016 (1999)), Faskhoudi et al. (Faskhoudi et al., Pathol. Res. Pract., 233: 153851 (2022)), Liao et al. (Liao et al., Endocrine-Related Cancer, 7(3): 143-164 (2000)), Schneider et al. (Schneider et al., EJNMMIRes., 11(1): 104 (2021)), Shrestha et al. (Shrestha et al., Front. Oncol., 11: 694320 (2021)), Zinmmierman et al. (Zimmerman et al., Cancer Discov., 8(3): 320-335 (2018)), Feng et al. (Feng et al., Ther. Adv. Med. Oncol., 12: 1-16 (2020)), Tang et al. (Tang et al., Cancer Lett., 273(1): 35-43 (2009)), Shroff et al. (Shroff et al., PNAS, 112(21): 6539-6544 (2015)), Durbin et al. (Durbin et al., Cancer Res., 80 (14_Supplement): B10 (2020)), and Chanvorachote et al. (Chanvorachote et al., Anticancer Research, 40: 609-618 (2020)), the disclosures of which, along with their references, are incorporated herein in their entirety.

Because of its broad pathogenic significance, MYC is an important target for cell proliferation diseases and disorders such as cancer, as well as other diseases characterized by gain-of-function of MYC. Studies show that for many cancer types, such as MYC-driven cancers, reducing the expression and/or activity of MYC leads to significant slowing of tumor growth, decrease in tumor size (i.e., tumor regression), and/or decreased metastasis in multiple models (e.g., cancer cell lines, animal models such as cell-line derived xenograft (CDX) models and patient-derived xenograft (PDX) models, etc.). Accordingly, there is a need to discover modulators that are capable of reducing or inhibiting the expression and/or activity of MYC, which are useful as therapeutic agents, as well as research tools.

Compared to other types of modulators (e.g., nucleic acids, siRNA, antisense oligonucleotides, CRISPR, gene therapy, antibodies, etc.), small molecule compounds offer distinct advantages such as, for example, ease of administration (most can be administered orally), ability to cross cell membranes to reach intracellular targets, tunability to allow for systemic distribution with or without distribution in the central nervous system (CNS), ability to engage biological targets via various modes of action, and/or lower cost of development and manufacturing in most cases. However, MYC has been a challenging target and is currently regarded as “undruggable” by small molecule compounds, as the MYC protein lacks pockets or grooves that could serve as good binding sites for small molecules. Small molecule compounds known in the art to target MYC often do so indirectly (e.g., inhibitors of the MYC-MAX protein-protein interaction) and lack the potency and appropriate pharmacokinetic properties for in vivo applications.

Thus, there is a need in the art for better means of reducing the expression and/or activity of MYC, as well as more effective therapies to treat, prevent, or ameliorate cell proliferation diseases and disorders such as cancer. The present disclosure addresses this and other unfulfilled needs in the art. A series of small molecule compounds and pharmaceutical compositions thereof, as well as methods of use thereof, for reducing the expression and/or activity of MYC in a cell, an animal or human subject are disclosed. Such compositions, systems, and methods are useful to treat, prevent, or ameliorate diseases, particularly cell proliferation diseases and disorders such as cancer, as well as other diseases characterized by gain-of-function of MYC.

### SUMMARY

The present invention relates to compounds of formula (I):

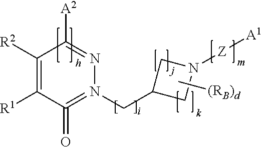

wherein A¹, A², R¹, R², R_{B}, Z, d, h, i, j, k and m, are as described herein, and pharmaceutically acceptable salts thereof.

Compositions are described comprising a compound of formula (I) disclosed herein, as well as pharmaceutical compositions or medicaments thereof, which are useful to treat, prevent, or ameliorate diseases, including cell proliferation diseases and disorders such as cancer, in particular MYC-driven cancer. Also described are methods of use of such compositions to treat, prevent, or ameliorate diseases, including cell proliferation diseases and disorders such as cancer, in particular MYC-driven cancer. Compositions comprising conjugates and complexes of a compound of formula (I) are also described, which are also useful in the methods described herein.

Methods are described comprising the use of a compound of formula (I) disclosed herein, or pharmaceutical compositions thereof, for the reduction or inhibition of MYC expression or activity. Methods are also described comprising the use of a compound of formula (I) disclosed herein, or pharmaceutical compositions thereof, for achieving one or more phenotypic outcomes such as, for example, decrease in cancer cell growth or proliferation, decrease in cancer cell viability, decrease in apoptosis, decrease in tumor volume (i.e., tumor regression), decrease in cancer metastasis, increase in animal or human subject survival, or other desired outcome with respect to particular phenotypes (e.g., body weight, metabolism, etc.). Related medicaments, kits, and methods of delivery of such compositions are described.

Methods are also described for the development, manufacture, and/or synthesis of a compound of formula (I) disclosed herein, as well as pharmaceutical compositions thereof. Furthermore, methods for diagnostics and testing comprising detecting MYC expression or activity levels, as well as compositions comprising kits for diagnostics and testing, are described herein.

Other features and advantages of the invention will be apparent from the detailed description and the examples that follow.

### DETAILED DESCRIPTION

The following description is presented to enable one of ordinary skill in the art to make and use the invention and is provided in the context of a patent application and its requirements. Various modifications to the example embodiments and the genetic principles and features described herein will be readily apparent. The example embodiments are mainly described in terms of particular processes and systems provided in particular implementations. However, the processes and systems will operate effectively in other implementations. Phrases such as “example embodiment”, “one embodiment”, and “another embodiment” can refer to the same or different embodiments.

The example embodiments will be described with respect to methods and compositions having certain components. However, the methods and compositions can include more or less components than those shown, and variations in the arrangement and type of the components can be made without departing from the scope of the invention.

The example embodiments will also be described in the context of methods having certain steps. However, the methods and compositions operate effectively with additional steps and steps in different orders that are not inconsistent with the example embodiments. Thus, the present invention is not intended to be limited to the embodiments shown but is to be accorded the widest scope consistent with the principles and features described herein and as limited only by appended claims.

Unless expressly stated, the terms used herein are intended to have the plain and ordinary meaning as understood by those of ordinary skill in the art. The following definitions are intended to aid the reader in understanding the present invention but are not intended to vary or otherwise limit the meaning of such terms unless specifically indicated. All publications mentioned herein are incorporated by reference for the purpose of describing and disclosing the formulations and processes that are described in the publication and which might be used in connection with the presently described invention.

Those skilled in the art will recognize or be able to ascertain using no more than routine experimentation, many equivalents to the specific embodiments of the invention described herein in the detailed description and FIGURES. Such equivalents are intended to be encompassed by the claims.

For simplicity, in the present document certain embodiments are described with respect to use of certain methods. It will become apparent to one skilled in the art upon reading this disclosure that the invention is not intended to be limited to a specific use and can be used in a wide array of implementations.

For clarity of disclosure, and not by way of limitation, the detailed description of the invention is divided into subsections that describe or illustrate certain features, embodiments, or applications of the present invention.

### General Definitions

Unless defined otherwise, all technical and scientific terms used herein have the same meaning as commonly understood by the ordinary person skilled in the art to which the embodiments pertain.

All publications, patent applications, patents, and other references mentioned herein are incorporated by reference in their entirety.

The term “optional” or “optionally” denotes that a subsequently described event or circumstance can but need not occur, and that the description includes instances where the event or circumstance occurs and instances in which it does not.

It should be noted that as used herein and in the appended claims, the singular forms “a”, “and”, and “the” include plural referents unless the context clearly dictates otherwise. Thus, for example, reference to the effect of “a compound” can refer to the effect of one or more compounds, and reference to “a method” includes reference to equivalent steps and processes known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those limits are also included in the invention.

“Nucleobases”, or “bases”, are used interchangeably and refer to nitrogen-containing compounds that form nucleosides, which in turn are components of nucleotides. The five primary or natural nucleobases are adenine (A), cytosine (C), guanine (G), thymine (T), and uracil (U). Other nucleobases, such as synthetic or modified nucleobases, are included herein and detailed below.

“Nucleotide” refers to a compound comprising a nucleoside and a linkage group, commonly a phosphate linkage group. Nucleotides include both natural and modified nucleotides.

“Motif” refers to a region or subsequence within the sequence of an oligonucleotide, or polypeptide, that has a specific functional or biological significance. Examples of motifs include nucleobase sequences within an oligonucleotide, such as DNA or RNA, which are recognized by a DNA or RNA-binding protein, or by functional RNAs (e.g., miRNAs). Other examples of motifs include nucleobase sequences within an RNA that are responsible for a specific function of the RNA, or amino acid sequences within a polypeptide that are responsible for a specific function of the polypeptide. A motif can also refer to a target site for a modulator on a DNA, RNA or polypeptide target.

“Nucleic acid sequence”, “nucleobase sequence”, “nucleotide sequence”, or simply “sequence” is used interchangeably and refer to the sequence of nucleobases on a nucleic acid molecule or oligonucleotide. A nucleic acid molecule can refer to a deoxyribonucleic acid (DNA) molecule or ribonucleic acid (RNA) molecule.

The term “gene” as used herein, refers to a DNA sequence that is transcribed to mRNA and subsequently translated to a polypeptide, and/or a DNA sequence that is transcribed to a functional RNA that is not translated to a polypeptide.

The term “RNA” as used herein, refers to a ribonucleic acid molecule. The process of transcription initially results in the formation of precursor mRNA (pre-mRNA). In the case of protein-coding genes, pre-mRNA is subsequently processed into mature mRNA by splicing to remove introns, as well as addition of a 5′ cap and poly-A tail. Mature mRNA is used as a template by ribosomes for translation into polypeptides. The term “RNA” as used herein, includes pre-mRNA (sometimes also referred to as heterogeneous nuclear RNA), mature mRNA, as well as RNA in any stage of processing. The term “RNA” as used herein, includes coding RNAs that are translated to polypeptides, and non-coding RNAs (e.g., miRNAs, tRNAs, rRNAs, etc.).

“Polypeptide”, “oligopeptide”, “peptide” and “protein” are used interchangeably and refer to a polymer of two or more amino acids.

“Oligonucleotide” refers to a polymer comprising two or more nucleotides.

An “allele”, also referred to as a “variant”, or “polymorphism”, refers to one of at least two different nucleotide sequence variations at a given position (locus) in the genome. Thus, a specific allele of a polymorphic site refers to a specific version of the sequence with respect to a polymorphic site. A “variant” or “polymorphism” can also refer to a specific allele of a polymorphic site that differs from a reference genome.

“Polymorphic marker”, also referred to as “polymorphic site” or simply as “marker”, refers to a genomic site with at least two sequence variants, or at least two alleles. Thus, genetic association with a polymorphic marker, refers to association with at least one specific allele of that polymorphic marker. “A marker” can also refer to a specific allele of a polymorphic marker. A polymorphic marker can refer to any type of sequence variation found in the genome, including but not limited to single nucleotide polymorphisms (SNPs), curated SNPs (cSNPs), insertions, deletions, copy number variations (CNVs), codon expansions, methylation status, translocations, duplications, repeat expansions, rearrangements, multi-base polymorphisms, splice variants, microsatellite polymorphisms etc. A “marker” can also refer to a “biomarker”.

A “single nucleotide polymorphism” or “SNP” is a type of variation of DNA where a single nucleotide at a specific location in a genome differs between two or more individuals, or two or more populations. Most SNPs have two alleles; in such cases, an individual is either homozygous for one allele at the polymorphic site, or heterozygous for both alleles.

An “insertion” or “deletion” is a variant with additional nucleotides or fewer nucleotides respectively compared to a reference DNA sequence.

A “microsatellite” is a type of polymorphic marker where there are multiple small repeats of bases that are 2-8 nucleotides in length.

The term “associated with” refers to and can be used interchangeably with “within”, or “correlated with”, or “in linkage disequilibrium with”, or “functionally related with”, or any combination of the terms. “Linkage disequilibrium” refers to the non-random association of alleles at different loci in a given population.

“Susceptibility” refers to the tendency, propensity or risk of an individual to develop a particular phenotype (e.g., a trait or a disease), or to being more or less able to resist developing a particular phenotype. The term encompasses decreased susceptibility to, or decreased risk of, or a protection against a disease. The term also encompasses an increased susceptibility to, or increased risk of developing, a disease.

The term “and/or” indicates “one or the other or both”. In other words, the term indicates that both or either of the items are involved.

The term “biomarker” refers to a biological molecule such as a protein, a polypeptide, a small molecule, a metabolite or a nucleic acid sequence that is associated with a phenotype such as a disease, and whose measurement can be used for determining a susceptibility to the disease, or prognosis for the disease, or diagnosis for the disease, or determining a response to a therapy for the disease.

The term “look-up table” is a table that links one form of data to another, or one or more forms of data to a predicted outcome (e.g., a trait, a disease, or other phenotype). Look-up tables can contain information about expression or activity levels of one or more targets, or one or more polymorphic markers, and a correlation between expression or activity levels of one or more targets, or between alleles for a polymorphic marker, and a particular phenotype (e.g., a trait or a disease).

A “computer-readable medium” is a medium for storage of information that is accessible by a computer interface that is custom-built or available commercially. Some examples of computer-readable media include, but are not limited to, optical storage media, magnetic storage media, memory, punch cards, or other commercially available media.

A “nucleic acid sample” refers to a DNA or RNA sample obtained from an individual. Nucleic acid samples can be obtained from any source that contains DNA or RNA, such as blood, saliva, tissue sample, cerebrospinal fluid, amniotic fluid etc.

A “sample” in general refers to any sample, such as a biological sample, obtained from an individual.

A “subject” may be interchangeable with “patient” or “individual” and refers to a living multi-cellular vertebrate organism, which includes both human and non-human mammals, unless otherwise indicated.

A “subject in need of treatment” may include a subject having a disease, disorder, or condition that is responsive to therapy with a compound disclosed herein. For example, a “subject in need of treatment” may include a subject having a cell proliferative disease, disorder, or condition such as cancer described herein. A “subject in need of treatment” may include a subject having a cell proliferative disease, disorder, or condition such as cancer that is associated with MYC expression or activity.

The term “therapeutic agent” refers to an agent that can be used for preventing, treating, or ameliorating symptoms associated with a disease.

The term “response to a therapeutic method”, “response to a therapy”, or “response to administration of a modulator” refers to the result of any kind of treatment on an individual, and includes beneficial, neutral, and adverse effects.

The term “therapeutically effective amount” refers to an amount of a therapeutic agent, which when administered alone or together with one or more additional therapeutic agents, induces the desired response, such as decreasing signs and symptoms associated with disease. Often, the therapeutically effective amount provides the desired response without causing significant side effects to the administered subject. An effective amount of a drug that is administered to a particular subject in a particular instance will not always be effective in treating the conditions/diseases described herein, even though such dosage is deemed to be a therapeutically effective amount by those of skill in the art.

The term “modulator” refers to a compound that affects the signaling, activity or expression of polypeptides or nucleic acid sequences (also referred to as “modulates”) and includes both activators and inhibitors. A modulator that increases or upregulates the signaling, activity or expression of polypeptides or nucleic acid sequences is referred to as an “activator”. A modulator that inhibits, reduces, decreases or downregulates the signaling, activity or expression of polypeptides or nucleic acid sequences is referred to as an “inhibitor”. “Modulation” refers to the act of modulating as defined above and can be performed with a modulator. Unless specified otherwise, “modulate” or “modulation” refers to the act of modulating as defined above, and includes both increasing or upregulating the signaling, activity or expression of polypeptides or nucleic acid sequences, as well as inhibiting, reducing, decreasing or downregulating the signaling, activity or expression of polypeptides or nucleic acid sequences.

The term “antisense modulator” refers to a modulator that affects the signaling, activity or expression of at least one nucleic acid sequence through some form of complementary binding or hybridization to the nucleic acid molecule. Common forms of antisense modulators include antisense oligonucleotides (ASOs) as well as nucleic acids used in the RNAi mechanism for gene modulation, including, but not limited to, miRNA, siRNA, and short hairpin RNA (shRNA), etc.

The term “amplification” or to “amplify” refers to increasing the number of copies of a sequence of nucleotides. An example of amplification is the “polymerase chain reaction”, in which a sample containing sequences of nucleotides is contacted with a pair of oligonucleotide primers. The primers hybridize with a nucleotide sequence, are extended under suitable conditions, and then are dissociated from the nucleotide sequence. This process is repeated to increase the number of copies of a sequence of nucleotides. Other methods can be used for amplification and are known to a person with ordinary skill in the art.

The term “isolated” refers to a purified, enriched or concentrated population of molecules. “Isolated” also refers to the act of enriching or concentrating a particular molecule, compound or complex such that its purity is increased.

The term “tissue” refers to an aggregate of cells that form a specific physiological function in an organism.

The term “delivery”, when used in the context of drugs, agents, or pharmaceutical compositions, refers to the administration of a drug, agent, or pharmaceutical composition to an assay mixture, a cell in culture, an animal, or a human subject or patient.

A “carrier”, also referred to as a “vehicle” or “excipient”, when used in the context of drugs, agents, or pharmaceutical compositions, is one or more molecules that is used to aid the delivery of one or more other molecules. Examples of a carrier include but are not limited to gelatin, cellulose, cellulose derivatives, polyvinylpyrrolidone, starch, sucrose and polyethylene glycol, etc.

“Cancer” refers to an abnormal growth of cells which tend to proliferate in an uncontrolled way and, in some cases, to metastasize. The term “cancer” or “cancers” include but are not limited to astrocytoma, breast cancer, brain lower grade glioma. Burkitt's lymphoma, cervical cancer, colorectal cancer (e.g., colon adenocarcinoma, rectum adenocarcinoma), esophageal carcinoma, gastric cancer (e.g., stomach adenocarcinoma), glioblastoma (e.g., glioblastoma multiforme), head and neck squamous cell carcinoma, leukemia (e.g., myeloid leukemia), liver cancer, lung cancer (e.g., non-small cell lung cancer, small cell lung carcinoma, lung squamous cell carcinoma), non-Burkitt's lymphoma, medullary thyroid carcinoma, medulloblastoma, melanoma (e.g., skin cutaneous melanoma, uveal melanoma), mesothelioma, multiple myeloma, neuroblastoma, osteosarcoma, ovarian cancer, pancreatic cancer, paraganglioma, pheochromocytoma, prostate cancer, renal cancer (e.g., clear cell renal cell carcinoma), retinoblastoma, rhabdomyosarcoma, and testicular cancer etc.

“Ameliorating” is the lessening of severity of a disease, as measured by at least one indicator of that disease. Indicators can be symptoms of that disease, or a marker associated with the disease and can be objectively or subjectively evaluated. In certain embodiments, to “ameliorate” can mean to slow, halt, or reverse the progression of a disease.

A “dose” is a specified unit of a pharmaceutical composition that is provided for administration. In some embodiments, dose can refer to a specified amount of a pharmaceutical composition that is administered over a period of time. The dose can refer to the total amount of the pharmaceutical composition administered, or the amount of pharmaceutical composition administered per unit of time.

### Chemical Definitions

New chemical entities and uses for chemical entities are disclosed herein. The chemical entities may be described using terminology known in the art and further discussed below.

The nomenclature used herein is based on IUPAC systematic nomenclature, or other nomenclature that is commonly used and understood by the ordinary person skilled in the art.

Any open valency appearing on a carbon, oxygen, sulfur, or nitrogen atom in the structures herein indicates the presence of a hydrogen, unless indicated otherwise.

The term “substituent” denotes an atom or a group of atoms replacing a hydrogen atom on the parent molecule.

The term “substituted” denotes that a specified group bears one or more substituents. Where any group can carry multiple substituents and a variety of possible substituents are provided, the substituents are independently selected and can be, but need not be, the same. The term “unsubstituted” means that the specified group bears no substituents. The term “optionally substituted” means that the specified group is unsubstituted or substituted by one or more substituents, independently chosen from the group of possible substituents.

When indicating the number of substituents, the term “one or more” refers to the range from one substituent to the highest possible number of substitutions, i.e., replacement of one hydrogen up to replacement of all hydrogens by substituents.

The term “moiety” refers to an atom or group of chemically bonded atoms that is attached to another atom or molecule by one or more chemical bonds thereby forming part of a molecule. For example, the variables A¹, A², R¹, R² R_{B}, and Z of formula (I) refer to moieties that are attached to the core structure of formula (I) by one or more covalent bonds.

As used herein, an asterisk “*”, a plus sign “+”, or a dotted line, may be used to designate the point of attachment for any radical group, moiety and/or substituent group.

The term “alkyl” refers to a straight-chain or branched saturated hydrocarbon group in all of its isomeric forms. In particular embodiments, alkyl refers to a straight or branched group of 1 to 12, 1 to 10, 1 to 8, 1 to 7, 1 to 6, 1 to 4, or 1 to 2 carbon atoms, which are also referred to herein as C₁₋₁₂-alkyl, C₁₋₁₀-alkyl, C₁₋₈-alkyl, C₁₋₇-alkyl, C₁₋₆-alkyl, C₁₋₄-alkyl, or C₁₋₂-alkyl, respectively. Some particular examples of alkyl groups include methyl, ethyl, propyl, isopropyl, n-butyl, iso-butyl, sec-butyl, or tert-butyl, etc.

The term “alkylene” refers to a diradical of an alkyl group (e.g., —(CH₂)ₙ— where n is an integer, such as an integer between 1 and 20). An exemplary alkylene group is —CH₂CH₂—. Other examples of alkylene groups include methylene, ethylene, propylene, 2-methylpropylene, butylene, 2-ethylbutylene, pentylene, hexylene, and the like.

The terms “halo”, “halogen”, and “halide” are used interchangeably herein and denote fluoro, chloro, bromo, or iodo. One particular example of halogen is fluoro.

The term “haloalkyl” refers to an alkyl group wherein at least one of the hydrogen atoms of the alkyl group is substituted by the same or different halogens. Examples of haloalkyl include monofluoromethyl (—CH₂F), difluoromethyl (—CHF₂), trifluoromethyl (—CF₃), 2,2,2-trifluoroethyl (—CH₂CF₃), and the like. “Perhaloalkyl” denotes an alkyl group where all hydrogen atoms of the alkyl group have been replaced by the same or different halogens.

The term “heteroalkyl” as used herein refers to an “alkyl” group in which at least one carbon atom has been replaced with a heteroatom (e.g., an O, N, or S atom). One example of a heteroalkyl group is an “alkoxy” group.

The term “alkenyl” as used herein refers to an unsaturated straight or branched hydrocarbon having at least one carbon-carbon double bond, such as a straight or branched group of 2 to 12, 2 to 10, 2 to 8, 2 to 7, 2 to 6, or 2 to 4 carbon atoms, which is also referred to herein as C₂₋₁₂-alkenyl, C₂₋₁₀-alkenyl, C₂₋₇-alkenyl, C₂₋₆-alkenyl, or C₂₋₄-alkenyl, respectively.

The term “alkynyl” as used herein refers to an unsaturated straight or branched hydrocarbon having at least one carbon-carbon triple bond, such as a straight or branched group of 2 to 12, 2 to 10, 2 to 8, 2 to 6, or 2 to 4 carbon atoms, which is also referred to herein as C₂₋₁₂-alkynyl, C₂₋₁₀-alkynyl, C₂₋₈-alkynyl, C₂₋₆-alkynyl, or C₂₋₄-alkynyl, respectively.

The terms “amine” and “amino” refer to a group of the formula —NR^{a}R^{c}, wherein R^{a} and R^{c} are independently hydrogen, alkyl, alkoxy, cycloalkyl, heterocycloalkyl, aryl, heteroaryl, heterocyclyl, or another group. Alternatively, R^{a} and R^{c}, together with the nitrogen to which they are attached, can form a heterocycloalkyl. The term “primary amino” denotes a group wherein both R^{a} and R^{c} are hydrogen. The term “secondary amino” denotes a group wherein R^{a} is hydrogen and R^{c} is a group other than hydrogen. The term “tertiary amino” denotes a group wherein both R^{a} and R^{c} are groups other than hydrogen. Examples of secondary and tertiary amino groups include methylamino, ethylamino, propylamino, isopropylamino, phenylamino, benzylamino, dimethylamino, diethylamino, dipropylamino, diisopropylamino, and the like.

The terms “alkoxy”, “alkoxyl” or “—O-alkyl” refer to an alkyl group as defined above, which is linked by an oxygen atom to the rest of the molecule. Examples of alkoxy groups include methoxy, ethoxy, tert-butoxy and the like. Where other moieties such as alkenyl, alkynyl, heteroalkyl etc., are linked by an oxygen atom to the rest of the molecule, it may be represented by —O-alkenyl, —O-alkynyl, —O-heteroalkyl etc., respectively.

The term “ether” refers to two hydrocarbons covalently linked by an oxygen atom.

The term “carbonyl” as used herein refers to the radical —C(O)—.

The term “oxo” refers to an oxygen atom doubly bonded to carbon or another element.

The term “carboxamido” as used herein refers to the radical —C(O)NRR^{f}, where R and R^{f} may be the same or different substituents. R and R^{f}, for example, may be independently alkyl, aryl, arylalkyl, cycloalkyl, formyl, haloalkyl, heteroaryl, or heterocyclyl etc.

The term “carboxy” or “carboxyl” as used herein refers to the radical —COOH or its corresponding salts, e.g. —COONa, etc.

The term “amide” or “amido” or “amidyl” as used herein refers to a radical of the form —R^{g}C(O)N(R^{d})—, —R^{g}C(O)N(R^{d})R^{e}—, —C(O)NR^{d}R^{e}, or —C(O)NH₂, wherein R^{g}, R^{d} and R^{e}, for example, are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro etc.

The term “bicyclic ring system” can refer to two rings fused to each other via a common single or double bond (annelated bicyclic ring system), or alternatively two rings fused together via a sequence of three or more common atoms (bridged bicyclic ring system), or alternatively two rings fused together via a common single atom (spiro bicyclic ring system). Bicyclic ring systems can be saturated, partially unsaturated, unsaturated, or aromatic. Bicyclic ring systems can comprise rings wherein one or more carbon atoms are substituted by the same or different heteroatoms selected from N, O and S.

The term “cycloalkyl” refers to a saturated cyclic, bicyclic, or bridged cyclic (e.g., adamantyl) hydrocarbon group of 3 to 12, 3 to 10, 3 to 8, 3 to 6, 4 to 8, or 4 to 6 carbons per ring, which is also referred to herein as C₃₋₁₂-cycloalkyl, C₃₋₁₀-cycloalkyl, C₃₋₈-cycloalkyl, C₃₋₆-cycloalkyl, C₄₋₈-cycloalkyl, or C₄₋₆-cycloalkyl respectively. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl, etc. In certain embodiments, the cycloalkyl group is not substituted, i.e., it is unsubstituted.

The term “partially unsaturated carbocyclyl” refers to a cyclic hydrocarbon that contains at least one double bond between ring atoms wherein at least one ring of the carbocyclyl is not aromatic. The partially unsaturated carbocyclyl may be characterized according to the number of ring carbon atoms. For example, the partially unsaturated carbocyclyl may contain 5 to 14, 5 to 12, 5 to 10, 5 to 8, or 5 to 6 ring carbon atoms, and accordingly be referred to as a 5-14-, 5-12-, 5-10-, 5-8-, or 5-6-membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Examples of partially unsaturated carbocyclyl groups also include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “cycloheteroalkyl” or “heterocycloalkyl” refers to a saturated or partly unsaturated, non-aromatic, mono- or polycyclic (e.g., having 2, 3 or 4 fused rings), or bridged cyclic ring system comprising 3 to 12, 3 to 10, 3 to 8, 3 to 6, 4 to 8, or 4 to 6 carbons per ring, wherein one or more of the ring-forming atoms is a heteroatom such as, for example, N, O, or S. Cycloheteroalkyl or heterocycloalkyl groups can include spirocycles. Examples of monocyclic saturated cycloheteroalkyl or heterocycloalkyl groups include aziridinyl, oxiranyl, azetidinyl, oxetanyl, pyrrolidinyl, tetrahydrofuranyl, tetrahydrothienyl, pyrazolidinyl, imidazolidinyl, oxazolidinyl, isoxazolidinyl, thiazolidinyl, isothiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, oxazepanyl, and the like. Examples of bicyclic saturated cycloheteroalkyl or heterocycloalkyl groups include 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, 3-thia-9-aza-bicyclo[3.3.1]nonyl, and the like. Examples of partly unsaturated cycloheteroalkyl or heterocycloalkyl groups include dihydrofuryl, imidazolinyl, dihydro-oxazolyl, tetrahydro-pyridinyl, dihydropyranyl, and the like. Also included in the definition of cycloheteroalkyl or heterocycloalkyl are moieties that have one or more aromatic rings fused (i.e., having a bond in common with) to the nonaromatic heterocyclic ring, for example phthalimidyl, naphthalimidyl, and benzo derivatives of heterocycles, such as for example 2,3-dihydrobenzofuryl, 1,3-benzodioxole, benzo-1,4-dioxane, and the like. A cycloheteroalkyl or heterocycloalkyl group having one or more fused aromatic rings can be attached though either the aromatic or non-aromatic portion. Also included in the definition of cycloheteroalkyl or heterocycloalkyl are moieties where one or more ring-forming atoms are substituted by 1 or 2 oxo or sulfido groups. In some embodiments, the cycloheteroalkyl or heterocycloalkyl group has between 1 to 6, 1 to 5, 1 to 4, 1 to 3, or 1 to 2 heteroatoms (e.g., N, O, or S). In some embodiments, the cycloheteroalkyl or heterocycloalkyl group contains 0, 1, 2, 3, 4, or 5 double bonds. In some embodiments, the cycloheteroalkyl or heterocycloalkyl group contains 0, 1 or 2 triple bonds.

The term “N-heterocycloalkyl” refers to a heterocycloalkyl group containing at least one nitrogen ring atom and where the point of attachment of the heterocycloalkyl radical to the rest of the molecule is through a nitrogen ring atom. Examples of N-heterocycloalkyl include 1,4-diazepanyl, hexahydropyrrolo[1,2-a]pyrazinyl, piperidinyl, piperazinyl, pyrrolidinyl, and the like, wherein the point of attachment of the heterocycloalkyl radical to the rest of the molecule is through a nitrogen ring atom.

The term “cycloalkylene” refers to a cycloalkyl group that is unsaturated at one or more ring bonds.

The term “aryl” refers to a carbocyclic aromatic group. Representative aryl groups include phenyl, naphthyl, anthracenyl, and the like. The term “aryl” also includes polycyclic ring systems having two or more carbocyclic rings in which two or more carbons are common to two adjoining rings (the rings are “fused rings”) wherein at least one of the rings is aromatic and the other ring(s) may be e.g., cycloalkyls, cycloalkenyls, cycloalkynyls, heterocycloalkyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6 to 10 membered ring structure.

The terms “heterocyclyl” and “heterocyclic group” refer to saturated, partially unsaturated, or aromatic ring systems, or a combination thereof, comprising 3 to 12, 3 to 10, 3 to 8, 3 to 6, 4 to 8, or 4 to 6 carbons per ring, wherein one or more of the ring-forming atoms is a heteroatom such as, for example, N, O, or S. As used herein, “heterocyclyl” includes “aryl” groups wherein at least 1 ring-forming atom is a heteroatom such as, for example, N, O, or S. In some embodiments, the heterocyclyl group has between 1 to 6, 1 to 5, 1 to 4, 1 to 3, or 1 to 2 heteroatoms (e.g., N, O, or S). The number of ring atoms in the heterocyclyl group can be specified using Cx-y nomenclature, wherein x and y are integers specifying the number of ring atoms. For example, a C₃₋₆ heterocyclyl group refers to a saturated or partially unsaturated 3- to 6-membered ring structure, wherein one or more ring atoms are heteroatoms (e.g., N, O, or S). The designation “C₃₋₆” indicates that the heterocyclic ring contains a total of between 3 to 6 ring atoms, inclusive of any heteroatoms that are ring atoms.

It is contemplated that the terms described herein can be appended to form chemically relevant combinations, such as for example, “carboxyheterocycloalkyl”, “arylalkylheteroaryl”, or “aninoalkylheterocyclyl”. The definitions described herein apply irrespective of whether the terms in question appear alone or in combination. The last member of the combination is the radical which is binding to the rest of the molecule. The other members of the combination are attached to the binding radical in reversed order in respect of the literal sequence. For example, the combination “aminoalkylheterocyclyl” refers to a heterocyclyl radical that is substituted by an alkyl, which is substituted by an amino group.

The terms “compound(s) of this invention”, “compound(s) of this disclosure”, “compound(s) of the disclosure” and “compound(s) of the present invention” refer to compounds as disclosed herein and stereoisomers, tautomers, solvates, and salts (e.g., pharmaceutically acceptable salts) thereof.

When the compounds of the invention are solids, it is understood by those skilled in the art that these compounds, and their solvates and salts, may exist in different solid forms, particularly different crystal forms, all of which are intended to be within the scope of the present invention and specified formulae.

The term “chiral center” denotes a carbon atom bonded to four nonidentical substituents. The term “chiral” refers to embodiments which are non-superimposable with their mirror image, while the term “achiral” refers to embodiments which are superimposable with their mirror image. Chiral molecules are optically active, i.e., they have the ability to rotate the plane of plane-polarized light.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al., Angew. Chem. Inter. Edit., 5: 385; errata 511 (1966)). The prefixes D and L or (+) and (−) are employed to designate the sign of rotation of plane-polarized light by the compound, with (−) or L designating that the compound is levorotatory. A compound prefixed with (+) or D is dextrorotatory.

Compounds of the present invention can have one or more chiral centers, and/or double bonds, and therefore exist as stereoisomers, such as geometric isomers, enantiomers or diastereomers. Whenever a chiral center, and/or double bond, is present in a chemical structure, it is intended that all stereoisomers associated with that chiral center, and/or double bond, are encompassed by the present invention. The term “stereoisomers” when used herein, encompasses all geometric isomers, enantiomers or diastereomers, and mixtures thereof.

Compounds of the present invention can exist in the form of optically pure enantiomers, or mixtures of enantiomers such as, for example, racemates, optically pure diastereoisomers, mixtures of diastereoisomers, diastereoisomeric racemates or mixtures of diastereoisomeric racemates. Also contemplated herein are compositions comprising, consisting essentially of, or consisting of an enantiopure compound, which composition may comprise, consist essentially of, or consist of at least about 10%, 20%, 30%, 40%, 50%, 60%, 70%, 80%, 90%, 95%, 96%, 97%, 98%, 99%, or 100% of a single enantiomer of a compound of formula (I) disclosed herein.

It is understood that graphical depictions of chemical structures, e.g., generic chemical structures, encompass all stereoisomeric forms of the specified compounds, unless indicated otherwise. The skilled artisan will recognize that a structure may denote a chiral center implicitly. Thus, throughout this specification, the compounds of the disclosure include all enantiomers, stereoisomers, racemic mixtures, and optically pure isomer forms.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

### Compounds

The present invention relates to compounds of formula (I):

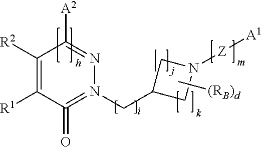

wherein
A¹ is hydrogen, C₁₋₇-alkyl, C₂₋₇-alkenyl, C₃₋₈-cycloalkyl, aryl, or heterocyclyl;
A² is hydrogen, C₁₋₇-alkyl, C₂₋₇-alkenyl, C₃₋₈-cycloalkyl, aryl, or heterocyclyl;
R¹ is hydrogen, halo, C₁₋₇-alkyl, C₂₋₇-alkenyl, C₃₋₈-cycloalkyl, aryl, or heterocyclyl;
R² is hydrogen, halo, C₁₋₇-alkyl, C₂₋₇-alkenyl, C₃₋₈-cycloalkyl, aryl, or heterocyclyl;
or A² and R² together form C₁₋₇-alkylene, or C₂₋₇-alkenylene;
Z is C₁₋₇-alkylene, C₂₋₇-alkenylene, —CH₂—, —(CH₂)₂—, —CH₂(CH)₂—, —C(═O)—, —C(═O)CH₂—, —C(═O)(CH₂)₂—, —C(═O)CH₂O—, —C(═O)(CH₂)₂O—, —C(═O)CH(CH₃)O—, —C(═O)O—, —C(═O)OCH₂—, —C(═O)O(CH₂)₂—, —C(═O)NH—, —C(═O)NHCH₂—, —C(═O)NH(CH₂)₂—, —S(═O)₂—, —S(═O)₂CH₂—, or —S(═O)₂(CH)₂—;
his 1, 2, or 3;
i is 0, 1 or 2;
j is 0, 1, 2 or 3;
k is 0, 1, 2 or 3;
m is 0 or 1;
R_{B} represents non-hydrogen substituent(s), wherein each R_{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein the R_{B} substituent(s), if any, is covalently attached to any ring atom(s) in place of hydrogen, provided the maximum valency of the ring atom(s) to which R_{B} is attached is not exceeded;
d is 0, 1, 2, 3, 4, 5, 6, 7, 8, 9, 10, 11, 12, or 13;
wherein C₁₋₇-alkyl, C₂₋₇-alkenyl, C₁₋₇-alkylene, C₂₋₇-alkenylene, C₃₋₈-cycloalkyl, aryl, and heterocyclyl, are each independently substituted or unsubstituted;
with the proviso that j and k are not both 0 in the same compound;
and pharmaceutically acceptable salts thereof.

Particular embodiments of the present invention are compounds of formula (I) and pharmaceutically acceptable salts, tautomers, N-oxides, and solvates thereof.

It is to be understood that every embodiment relating to a specific A¹, A², R¹, R², R_{B}, Z, d, h, i, j, k and m, as disclosed herein may be combined with any other embodiment relating to another A¹, A², R¹, R², R_{B}, Z, d, h, i, j, k and m, as disclosed herein.

A particular embodiment of the present invention relates to compounds of formula (I) wherein
A¹ is hydrogen, C₁₋₇-alkyl, C₂₋₇-alkenyl, C₃₋₈-cycloalkyl, aryl, or heterocyclyl;
A² is hydrogen, C₁₋₇-alkyl, C₂₋₇-alkenyl, C₃₋₈-cycloalkyl, aryl, or heterocyclyl;
R¹ is hydrogen, halo, C₁₋₇-alkyl, C₂₋₇-alkenyl, C₃₋₈-cycloalkyl, aryl, or heterocyclyl;
R² is hydrogen, halo, C₁₋₇-alkyl, C₂₋₇-alkenyl, C₃₋₈-cycloalkyl, aryl, or heterocyclyl;
or A² and R² together form C₁₋₇-alkylene, or C₂₋₇-alkenylene;
Z is C₁₋₇-alkylene, C₂₋₇-alkenylene, —CH₂—, —(CH₂)₂—, —CH₂(CH)₂—, —C(═O)—, —C(═O)CH₂—, —C(═O)(CH₂)₂—, —C(═O)CH₂O—, —C(═O)(CH₂)₂O—, —C(═O)CH(CH₃)O—, —C(═O)O—, —C(═O)OCH₂—, —C(═O)O(CH₂)₂—, —C(═O)NH—, —C(═O)NHCH₂—, —C(═O)NH(CH₂)₂—, —S(═O)₂—, —S(═O)₂CH₂—, or —S(═O)₂(CH)₂—;
his 1, 2, or 3;
i is 0, 1 or 2;
j is 0, 1, 2 or 3;
k is 0, 1, 2 or 3;
m is 0 or1;
R_{B} represents non-hydrogen substituent(s), wherein each R_{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein the R_{B} substituent(s), if any, is covalently attached to any ring atom(s) in place of hydrogen, provided the maximum valency of the ring atom(s) to which R_{B} is attached is not exceeded;
d is 0, 1, 2, 3, 4, 5, 6, 7, 8, 9, 10, 11, 12, or 13;
wherein C₁₋₇-alkyl, C₂₋₇-alkenyl, C₁₋₇-alkylene, C₂₋₇-alkenylene, C₃₋₈-cycloalkyl, aryl, and heterocyclyl, are each independently substituted or unsubstituted;
with the proviso that j and k are not both 0 in the same compound;
and pharmaceutically acceptable salts, tautomers, N-oxides, and solvates thereof.

A particular embodiment of the present invention relates to compounds of formula (I), wherein R¹ is hydrogen, halo, C₁₋₇-alkyl, C₂₋₇-alkenyl, C₃₋₈-cycloalkyl, aryl, or heterocyclyl, particularly hydrogen or C₁₋₇-alkyl.

A particular embodiment of the present invention relates to compounds of formula (I), wherein R² is hydrogen, halo, C₁₋₇-alkyl, C₂₋₇-alkenyl, C₃₋₈-cycloalkyl, aryl, or heterocyclyl, particularly hydrogen or C₁₋₇-alkyl.

A particular embodiment of the present invention relates to compounds of formula (I), wherein A² and R² together form C₁₋₇-alkylene, or C₂₋₇-alkenylene, particularly propylene or butylene.

A particular embodiment of the present invention relates to compounds of formula (I), wherein R_{B} is bromo, chloro, fluoro, or iodo, particularly fluoro.

A particular embodiment of the present invention relates to compounds of formula (I), wherein Z is C₁₋₇-alkylene, C₂₋₇-alkenylene, —CH₂—, —(CH₂)₂—, —CH₂(CH)₂—, —C(═O)—, —C(═O)CH₂—, —C(═O)(CH₂)₂—, —C(═O)CH₂O—, —C(═O)(CH₂)₂O—, —C(═O)CH(CH₃)O—, —C(═O)O—, —C(═O)OCH₂—, —C(═O)O(CH₂)₂—, —C(═O)NH—, —C(═O)NHCH₂—, —C(═O)NH(CH₂)₂—, —S(═O)₂—, —S(═O)₂CH₂—, or —S(═O)₂(CH)₂—, particularly —CH₂—, —(CH₂)₂—, —C(═O)—, —C(═O)CH₂—, or —C(═O)NH—.

A particular embodiment of the present invention relates to compounds of formula (I), wherein m is 0 or 1, particularly 0. For the avoidance of doubt, if m is 0, the Z moiety bridging A¹ and N in formula (I) is replaced by a direct single covalent bond between A¹ and N.

A particular embodiment of the present invention relates to compounds of formula (I), wherein m is 0 or 1, particularly 1.

A particular embodiment of the present invention relates to compounds of formula (I), wherein d is 0, 1, 2, 3, or 4, particularly 0, 1 or 2.

A particular embodiment of the present invention relates to compounds of formula (I), wherein h is 1, 2, or 3, particularly 1.

A particular embodiment of the present invention relates to compounds of formula (I), wherein i is 0, 1 or 2, particularly 0. For the avoidance of doubt, where i in —[CH₂]ᵢ— of formula (I) is 0, that particular —CH₂— linkage is replaced by a direct single covalent bond.

A particular embodiment of the present invention relates to compounds of formula (I), wherein i is 0, 1 or 2, particularly 1.

A particular embodiment of the present invention relates to compounds of formula (I), wherein j is 0, 1, 2, or 3, particularly 1.

A particular embodiment of the present invention relates to compounds of formula (I), wherein j is 0, 1, 2, or 3, particularly 2.

A particular embodiment of the present invention relates to compounds of formula (I), wherein k is 0, 1, 2, or 3, particularly 1.

A particular embodiment of the present invention relates to compounds of formula (I), wherein k is 0, 1, 2, or 3, particularly 2.

A particular embodiment of the present invention relates to compounds of formula (I), wherein h is 1, i is 1, j is 2, k is 2, and m is 0.

A particular embodiment of the present invention relates to compounds of formula (I), wherein one or more hydrogens is replaced with deuterium, which may confer useful properties to the compound such as, for example, to prolong the residence time of the active drug species in plasma to achieve greater efficacy, and/or to avoid adverse side effects.

A particular embodiment of the present invention relates to compounds of formula (I),
wherein A¹ is

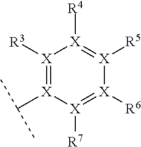

wherein
each X is independently C or N;
R³, R⁴, R⁵, R⁶ and R⁷ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R³, R⁴, R⁵, R⁶ or R⁷) attached to that particular X.

A particular embodiment of the present invention relates to compounds of formula (I), wherein R³ is hydrogen or C₁₋₇-alkyl, particularly hydrogen or methyl.

A particular embodiment of the present invention relates to compounds of formula (I), wherein R⁴ is hydrogen or C₁₋₇-alkyl, particularly hydrogen or methyl.

A particular embodiment of the present invention relates to compounds of formula (I), wherein R⁵ is hydrogen or C₁₋₇-alkyl, particularly hydrogen or methyl.

A particular embodiment of the present invention relates to compounds of formula (I), wherein R⁶ is hydrogen or C₁₋₇-alkyl, particularly hydrogen or methyl.

A particular embodiment of the present invention relates to compounds of formula (I), wherein R⁷ is hydrogen or C₁₋₇-alkyl, particularly hydrogen or methyl.

A particular embodiment of the present invention relates to compounds of formula (I), wherein one of R³, R⁴, R⁵, R⁶ or R⁷, is other than hydrogen.

A particular embodiment of the present invention relates to compounds of formula (I), wherein at least one of R³, R⁴, R⁵, R⁶ or R⁷, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A¹ is selected from the group of

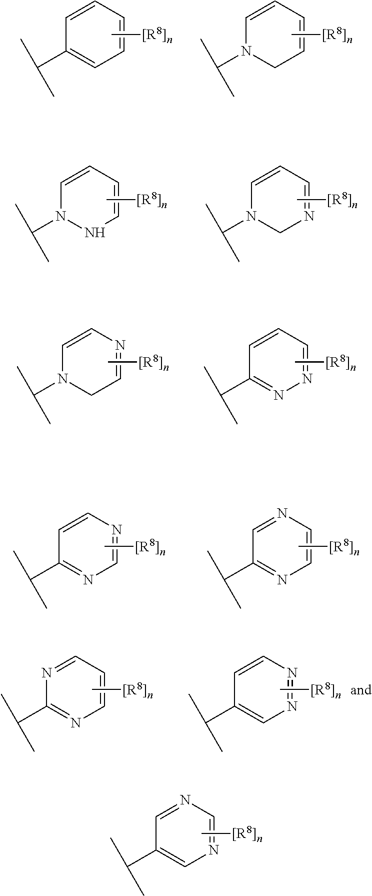

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A¹ in place of hydrogen, provided the maximum valency of the ring atom(s) to which R^{B} is attached is not exceeded;
n is 0, 1, 2, 3, 4, 5 or 6.

A particular embodiment of the present invention relates to compounds of formula (I), wherein n is 0.

A particular embodiment of the present invention relates to compounds of formula (I), wherein n is 1 or 2.

A particular embodiment of the present invention relates to compounds of formula (I), wherein A¹ is selected from the group of

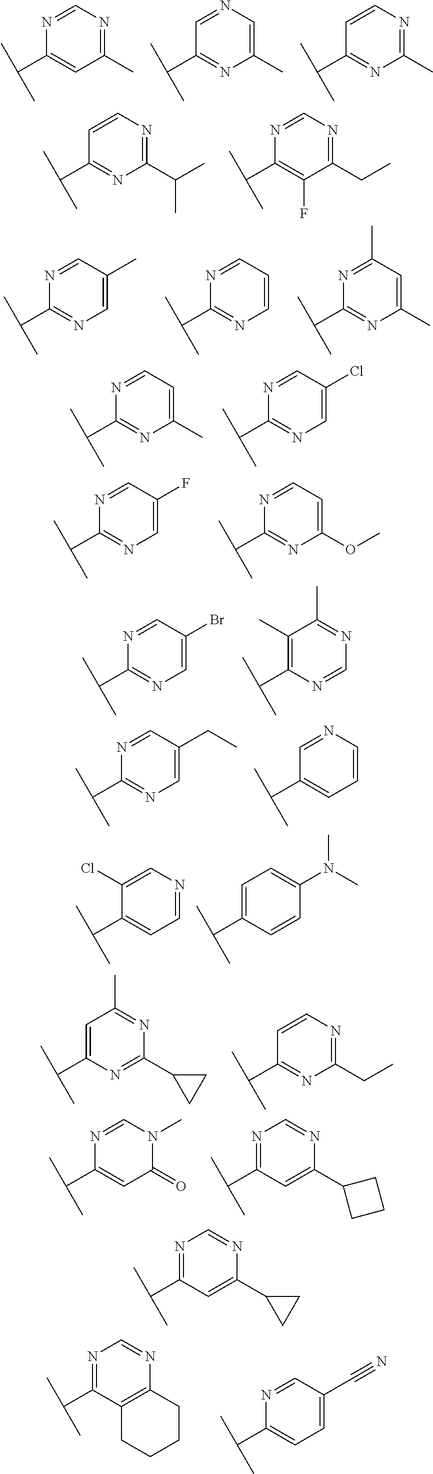

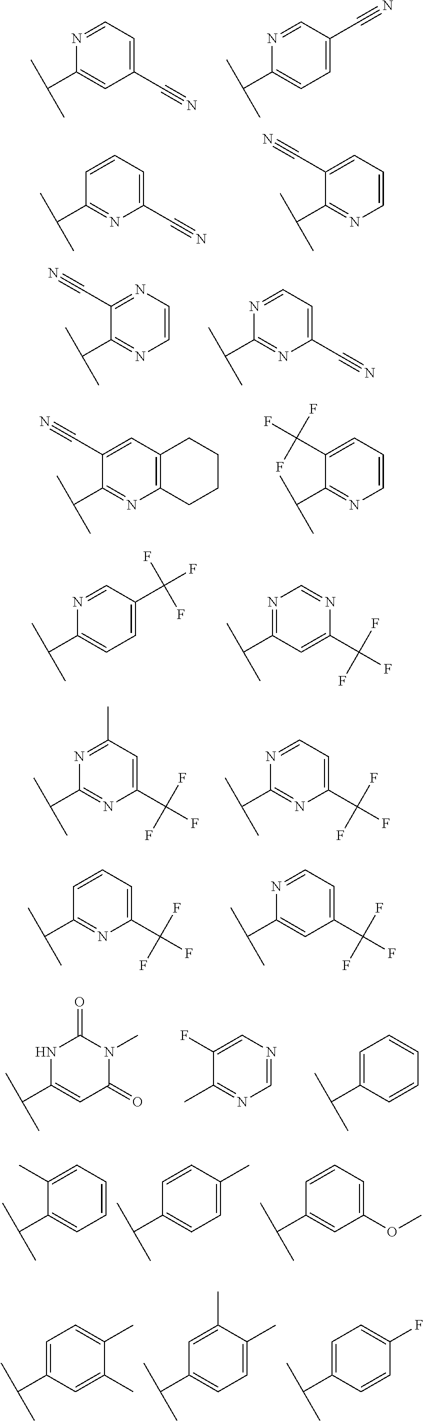

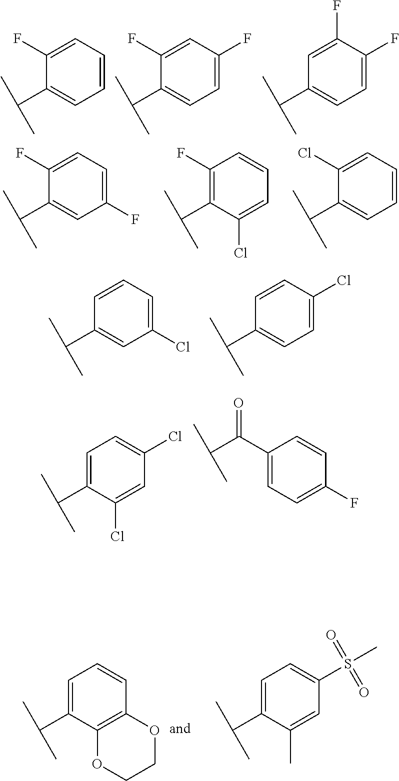

A particular embodiment of the present invention relates to compounds of formula (I),
wherein A¹ is

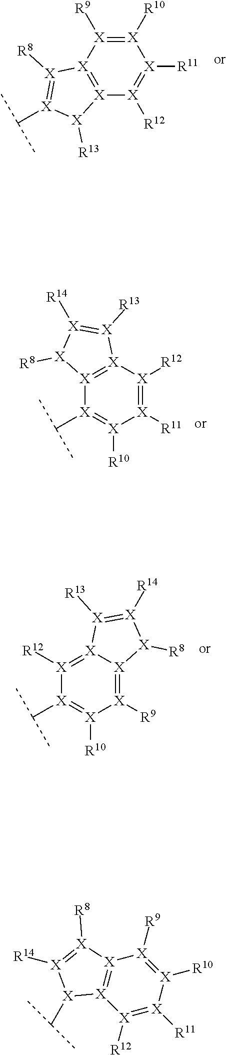

wherein
each X is independently C, N, O or S;
R⁸, R⁹, R¹⁰, R¹¹, R¹², R¹³, and R¹⁴ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R⁸, R⁹, R¹⁰, R¹¹, R¹², R¹³, or R¹⁴) attached to that particular X.

A particular embodiment of the present invention relates to compounds of formula (I), wherein R⁸ is hydrogen or C₁₋₇-alkyl, particularly hydrogen or methyl.

A particular embodiment of the present invention relates to compounds of formula (I), wherein R⁹ is hydrogen or C₁₋₇-alkyl, particularly hydrogen or methyl.

A particular embodiment of the present invention relates to compounds of formula (I), wherein R¹⁰ is hydrogen or C₁₋₇-alkyl, particularly hydrogen or methyl.

A particular embodiment of the present invention relates to compounds of formula (I), wherein R¹¹ is hydrogen or C₁₋₇-alkyl, particularly hydrogen or methyl.

A particular embodiment of the present invention relates to compounds of formula (I), wherein R¹² is hydrogen or C₁₋₇-alkyl, particularly hydrogen or methyl.

A particular embodiment of the present invention relates to compounds of formula (I), wherein R¹³ is hydrogen or C₁₋₇-alkyl, particularly hydrogen or methyl.

A particular embodiment of the present invention relates to compounds of formula (I), wherein R¹⁴ is hydrogen or C₁₋₇-alkyl, particularly hydrogen or methyl.

A particular embodiment of the present invention relates to compounds of formula (I), wherein one of R⁸, R⁹, R¹⁰, R¹¹, R¹², R¹³, or R¹⁴, is other than hydrogen.

A particular embodiment of the present invention relates to compounds of formula (I), wherein at least one of R⁸, R⁹, R¹⁰, R¹¹, R¹², R¹³, or R¹⁴, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A¹ is selected from the group of

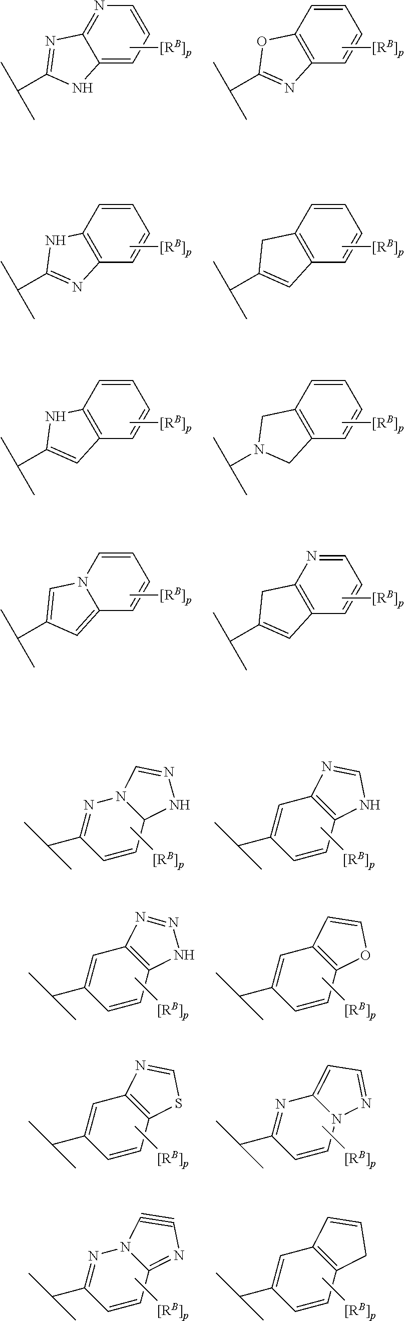

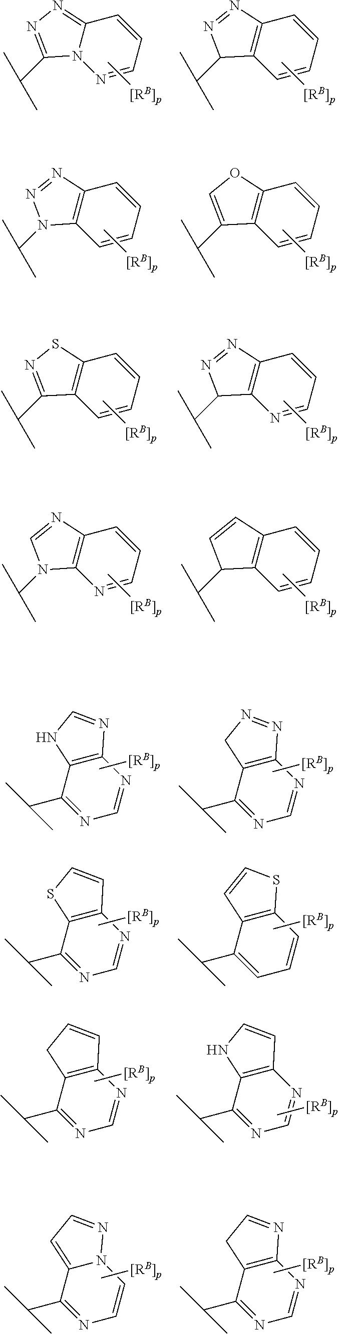

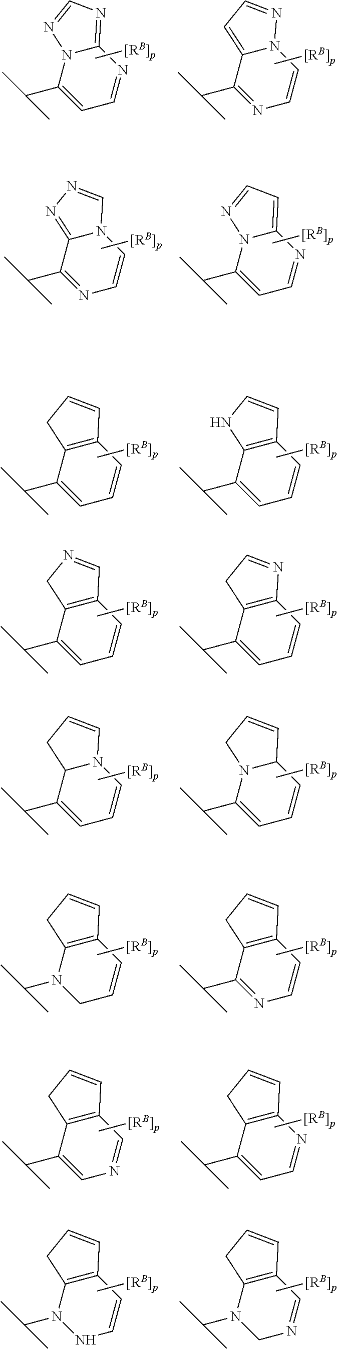

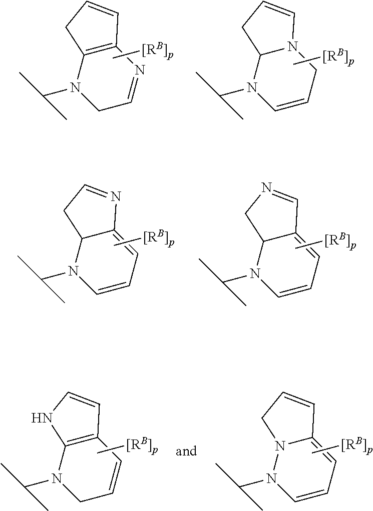

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A¹ in place of hydrogen, including for either or both 5-membered and 6-membered rings, provided the maximum valency of the ring atom(s) to which R^{B} is attached is not exceeded;
p is 0, 1, 2, 3, 4, 5, 6, 7, or 8.

A particular embodiment of the present invention relates to compounds of formula (I), wherein p is 0.

A particular embodiment of the present invention relates to compounds of formula (I), wherein p is 1 or 2.

A particular embodiment of the present invention relates to compounds of formula (I), wherein A¹ is selected from the group of

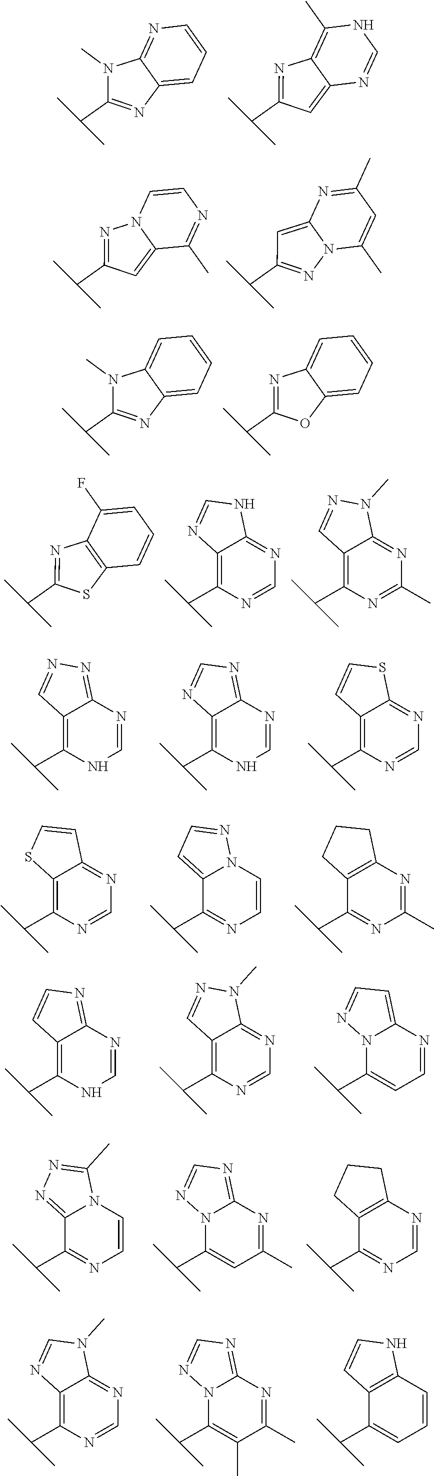

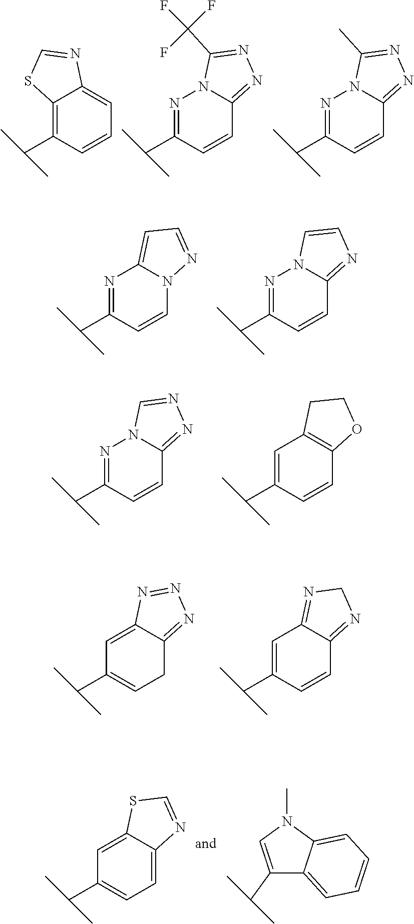

A particular embodiment of the present invention relates to compounds of formula (I),

wherein A¹ is

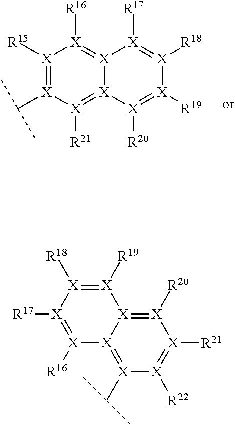

wherein
each X is independently C, N, O, or S;
R¹⁵, R¹⁶, R¹⁷, R¹⁸, R¹⁹, R²⁰, R²¹, and R²² are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R¹⁵, R¹⁶, R¹⁷, R¹⁸, R¹⁹, R²⁰, R²¹, or R²²) attached to that particular X.

A particular embodiment of the present invention relates to compounds of formula (I), wherein R¹⁵ is hydrogen or C₁₋₇-alkyl, particularly hydrogen or methyl.

A particular embodiment of the present invention relates to compounds of formula (I), wherein R¹⁶ is hydrogen or C₁₋₇-alkyl, particularly hydrogen or methyl.

A particular embodiment of the present invention relates to compounds of formula (I), wherein R¹⁷ is hydrogen or C₁₋₇-alkyl, particularly hydrogen or methyl.

A particular embodiment of the present invention relates to compounds of formula (I), wherein R¹⁸ is hydrogen or C₁₋₇-alkyl, particularly hydrogen or methyl.

A particular embodiment of the present invention relates to compounds of formula (I), wherein R¹⁹ is hydrogen or C₁₋₇-alkyl, particularly hydrogen or methyl.

A particular embodiment of the present invention relates to compounds of formula (I), wherein R²⁰ is hydrogen or C₁₋₇-alkyl, particularly hydrogen or methyl.

A particular embodiment of the present invention relates to compounds of formula (I), wherein R²¹ is hydrogen or C₁₋₇-alkyl, particularly hydrogen or methyl.

A particular embodiment of the present invention relates to compounds of formula (I), wherein R²² is hydrogen or C₁₋₇-alkyl, particularly hydrogen or methyl.

A particular embodiment of the present invention relates to compounds of formula (I), wherein one of R¹⁵, R¹⁶, R¹⁷, R¹⁸, R¹⁹, R²⁰, R²¹, or R²², is other than hydrogen.

A particular embodiment of the present invention relates to compounds of formula (I), wherein at least one of R¹⁵, R¹⁶, R¹⁷, R¹⁸, R¹⁹, R²⁰, R²¹, or R²², is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A¹ is selected from the group of

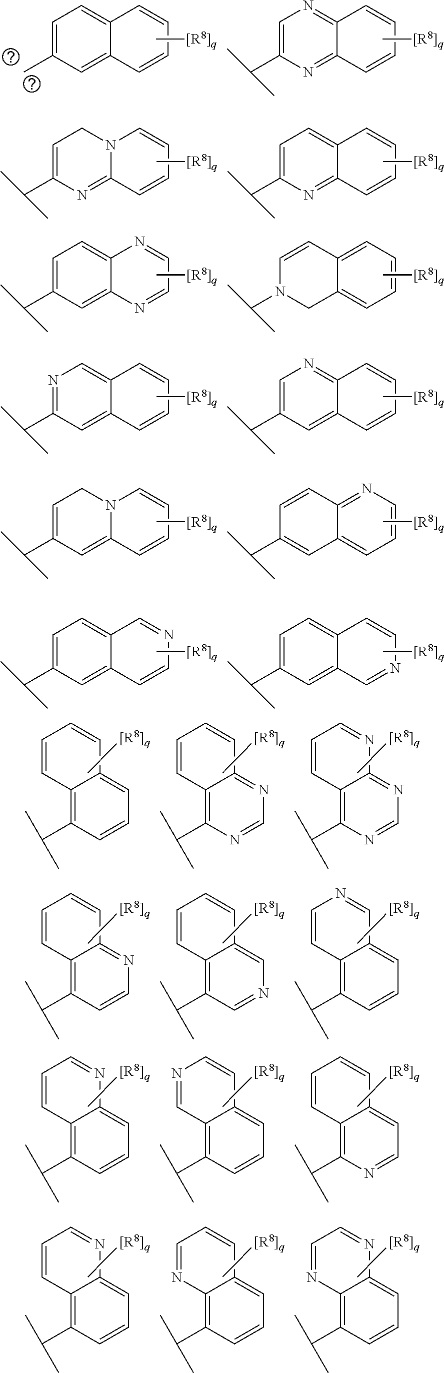

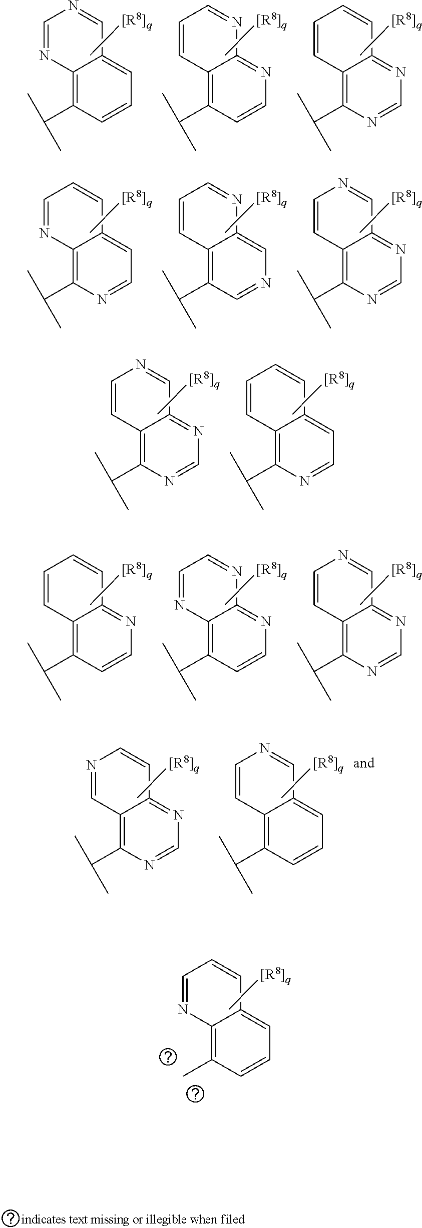

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R_{B} substituent(s) can be covalently attached to any ring atom(s) of A¹ in place of hydrogen, including for either or both of the 6-membered rings, provided the maximum valency of the ring atom(s) to which R^{B} is attached is not exceeded;
q is 0, 1, 2, 3, 4, 5, 6, 7, or 8.

A particular embodiment of the present invention relates to compounds of formula (I), wherein q is 0.

A particular embodiment of the present invention relates to compounds of formula (I), wherein q is 1 or 2.

A particular embodiment of the present invention relates to compounds of formula (I), wherein A¹ is selected from the group of

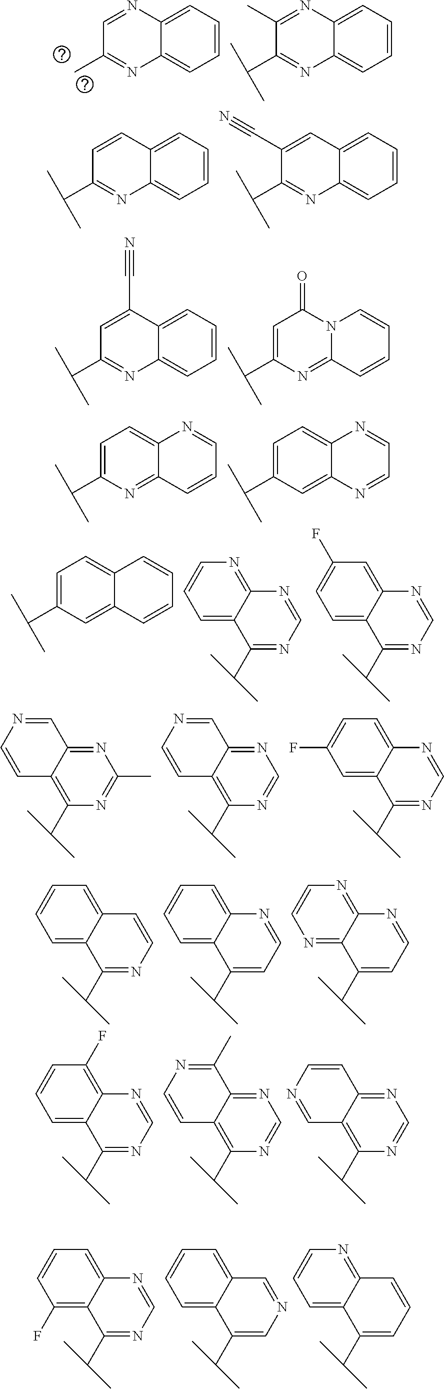

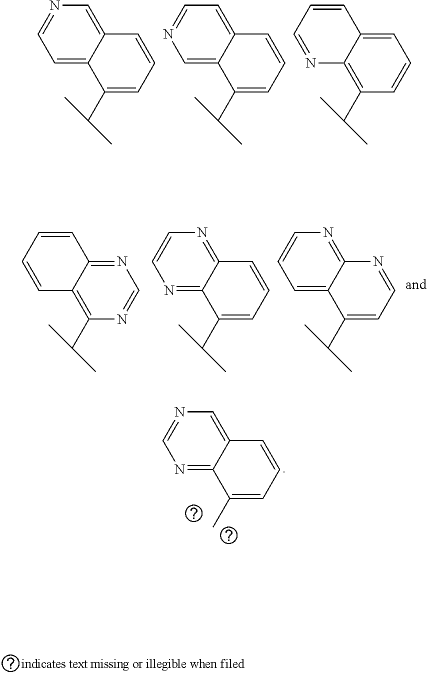

A particular embodiment of the present invention relates to compounds of formula (I),

wherein A¹ is

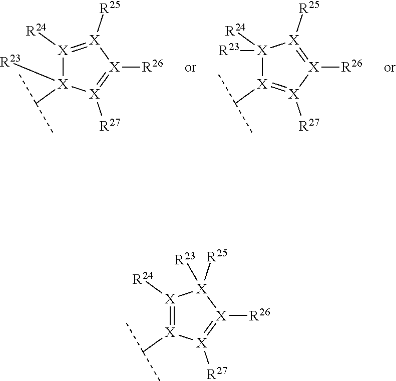

wherein
each X is independently C, N, O or S;
R²³, R²⁴, R²⁵, R²⁶, and R²⁷ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R²³, R²⁴, R²⁵, R²⁶, or R²⁷) attached to that particular X.

A particular embodiment of the present invention relates to compounds of formula (I), wherein R²³ is hydrogen or C₁₋₇-alkyl, particularly hydrogen or methyl.

A particular embodiment of the present invention relates to compounds of formula (I), wherein R²⁴ is hydrogen or C₁₋₇-alkyl, particularly hydrogen or methyl.

A particular embodiment of the present invention relates to compounds of formula (I), wherein R²⁵ is hydrogen or C₁₋₇-alkyl, particularly hydrogen or methyl.

A particular embodiment of the present invention relates to compounds of formula (I), wherein R²⁶ is hydrogen or C₁₋₇-alkyl, particularly hydrogen or methyl.

A particular embodiment of the present invention relates to compounds of formula (I), wherein R²⁷ is hydrogen or C₁₋₇-alkyl, particularly hydrogen or methyl.

A particular embodiment of the present invention relates to compounds of formula (I), wherein one of R²³, R²⁴, R²⁵, R²⁶, or R²⁷, is other than hydrogen.

A particular embodiment of the present invention relates to compounds of formula (I), wherein at least one of R²³, R²⁴, R²⁵, R²⁶, or R²⁷, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A¹ is selected from the group of

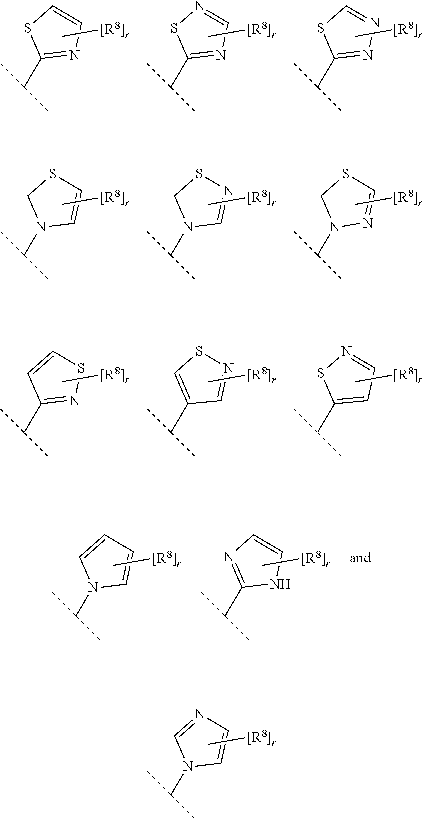

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A¹ in place of hydrogen, provided the maximum valency of the ring atom(s) to which R^{B} is attached is not exceeded;
r is 0, 1, 2, 3, 4, or 5.

A particular embodiment of the present invention relates to compounds of formula (I), wherein r is 0.

A particular embodiment of the present invention relates to compounds of formula (I), wherein r is 1 or 2.

A particular embodiment of the present invention relates to compounds of formula (I), wherein A¹ is selected from the group of

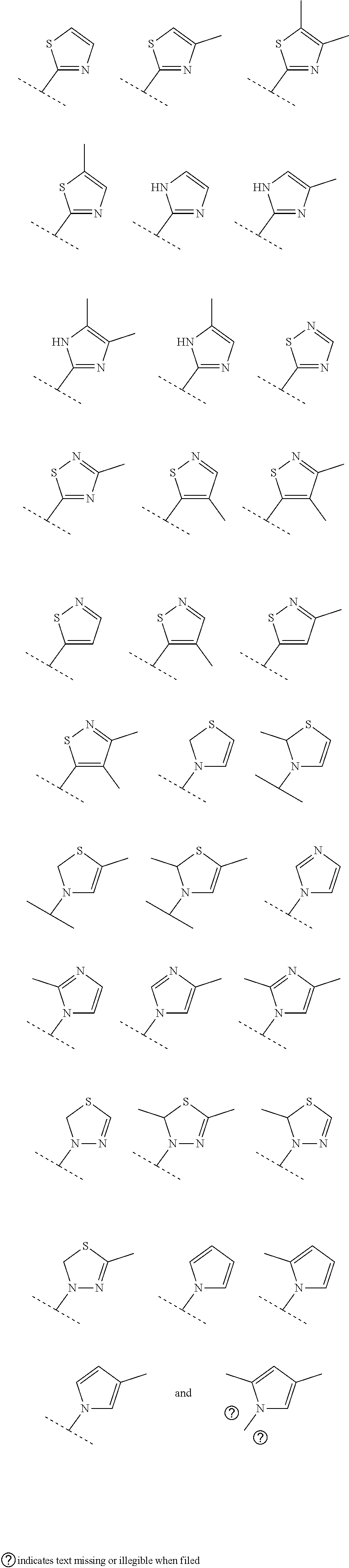

A particular embodiment of the present invention relates to compounds of formula (I),

wherein A¹ is

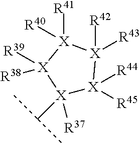

wherein
each X is independently C, N, O or S;
R³⁷, R³⁸, R³⁹, R⁴⁰, R⁴¹, R⁴², R⁴³, R⁴⁴, and R⁴⁵ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R³⁷, R³⁸, R³⁹, R⁴⁰, R⁴¹, R⁴², R⁴³, R⁴⁴, or R⁴⁵) attached to that particular X.

A particular embodiment of the present invention relates to compounds of formula (I), wherein R³⁷ is hydrogen or C₁₋₇-alkyl, particularly hydrogen or methyl.

A particular embodiment of the present invention relates to compounds of formula (I), wherein R³⁸ is hydrogen or C₁₋₇-alkyl, particularly hydrogen or methyl.

A particular embodiment of the present invention relates to compounds of formula (I), wherein R³⁹ is hydrogen or C₁₋₇-alkyl, particularly hydrogen or methyl.

A particular embodiment of the present invention relates to compounds of formula (I), wherein R⁴⁰ is hydrogen or C₁₋₇-alkyl, particularly hydrogen or methyl.

A particular embodiment of the present invention relates to compounds of formula (I), wherein R⁴¹ is hydrogen or C₁₋₇-alkyl, particularly hydrogen or methyl.

A particular embodiment of the present invention relates to compounds of formula (I), wherein R⁴² is hydrogen or C₁₋₇-alkyl, particularly hydrogen or methyl.

A particular embodiment of the present invention relates to compounds of formula (I), wherein R⁴³ is hydrogen or C₁₋₇-alkyl, particularly hydrogen or methyl.

A particular embodiment of the present invention relates to compounds of formula (I), wherein R⁴⁴ is hydrogen or C₁₋₇-alkyl, particularly hydrogen or methyl.

A particular embodiment of the present invention relates to compounds of formula (I), wherein R⁴⁵ is hydrogen or C₁₋₇-alkyl, particularly hydrogen or methyl.

A particular embodiment of the present invention relates to compounds of formula (I), wherein one of R³⁷, R³, R³⁹, R⁴⁰, R⁴¹, R⁴², R⁴³, R⁴⁴, or R⁴⁵, is other than hydrogen.

A particular embodiment of the present invention relates to compounds of formula (I), wherein at least one of R³⁷, R³⁸, R³⁹, R⁴⁰, R⁴¹, R⁴², R⁴³, R⁴⁴, or R⁴⁵, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A¹ is selected from the group of

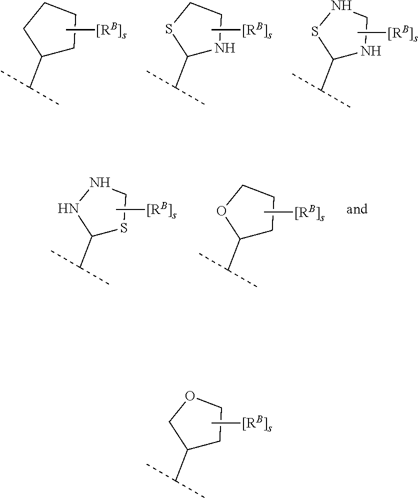

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A¹ in place of hydrogen, provided the maximum valency of the ring atom(s) to which R_{B} is attached is not exceeded;
s is 0, 1, 2, 3, 4, 5, 6, 7, 8, or 9.

A particular embodiment of the present invention relates to compounds of formula (I), wherein s is 0.

A particular embodiment of the present invention relates to compounds of formula (I), wherein s is 1 or 2.

A particular embodiment of the present invention relates to compounds of formula (I), wherein A¹ is selected from the group of

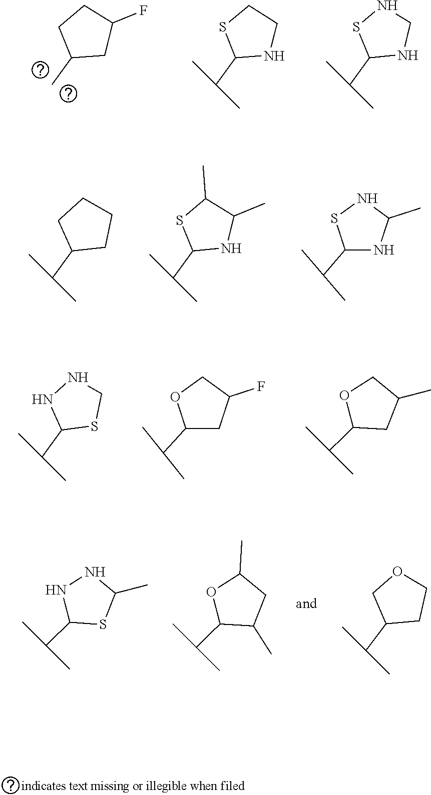

A particular embodiment of the present invention relates to compounds of formula (I),

wherein A¹ is

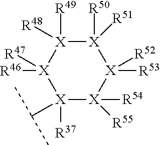

wherein
each X is independently C, N, O or S;
R³⁷, R⁴⁶, R⁴⁷, R⁴⁸, R⁴⁹, R⁵⁰, R⁵¹, R⁵², R⁵³, R⁵⁴, and R⁵⁵ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R³⁷, R⁴⁶, R⁴⁷, R⁴⁸, R⁴⁹, R⁵⁰, R⁵, R⁵², R⁵, R⁵⁴, or R⁵⁵) attached to that particular X.

A particular embodiment of the present invention relates to compounds of formula (I), wherein R⁴⁶ is hydrogen or C₁₋₇-alkyl, particularly hydrogen or methyl.

A particular embodiment of the present invention relates to compounds of formula (I), wherein R⁴⁷ is hydrogen or C₁₋₇-alkyl, particularly hydrogen or methyl.

A particular embodiment of the present invention relates to compounds of formula (I), wherein R⁴⁸ is hydrogen or C₁₋₇-alkyl, particularly hydrogen or methyl.

A particular embodiment of the present invention relates to compounds of formula (I), wherein R⁴⁹ is hydrogen or C₁₋₇-alkyl, particularly hydrogen or methyl.

A particular embodiment of the present invention relates to compounds of formula (I), wherein R⁵¹ is hydrogen or C₁₋₇-alkyl, particularly hydrogen or methyl.

A particular embodiment of the present invention relates to compounds of formula (I), wherein R⁵¹ is hydrogen or C₁₋₇-alkyl, particularly hydrogen or methyl.

A particular embodiment of the present invention relates to compounds of formula (I), wherein R⁵² is hydrogen or C₁₋₇-alkyl, particularly hydrogen or methyl.

A particular embodiment of the present invention relates to compounds of formula (I), wherein R⁵³ is hydrogen or C₁₋₇-alkyl, particularly hydrogen or methyl.

A particular embodiment of the present invention relates to compounds of formula (I), wherein R⁵⁴ is hydrogen or C₁₋₇-alkyl, particularly hydrogen or methyl.

A particular embodiment of the present invention relates to compounds of formula (I), wherein R⁵⁵ is hydrogen or C₁₋₇-alkyl, particularly hydrogen or methyl.

A particular embodiment of the present invention relates to compounds of formula (I), wherein one of R³⁷, R⁴⁶, R⁴⁷, R⁴⁸, R⁴⁹, R⁵⁰, R⁵¹, R⁵², R⁵³, R⁵⁴, or R⁵⁵ is other than hydrogen.

A particular embodiment of the present invention relates to compounds of formula (I), wherein at least one of R³⁷, R⁴⁶, R⁴⁷, R⁴⁸, R⁴⁹, R⁵⁰, R⁵¹, R⁵², R⁵³, R⁵⁴, or R⁵⁵, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A¹ is selected from the group of

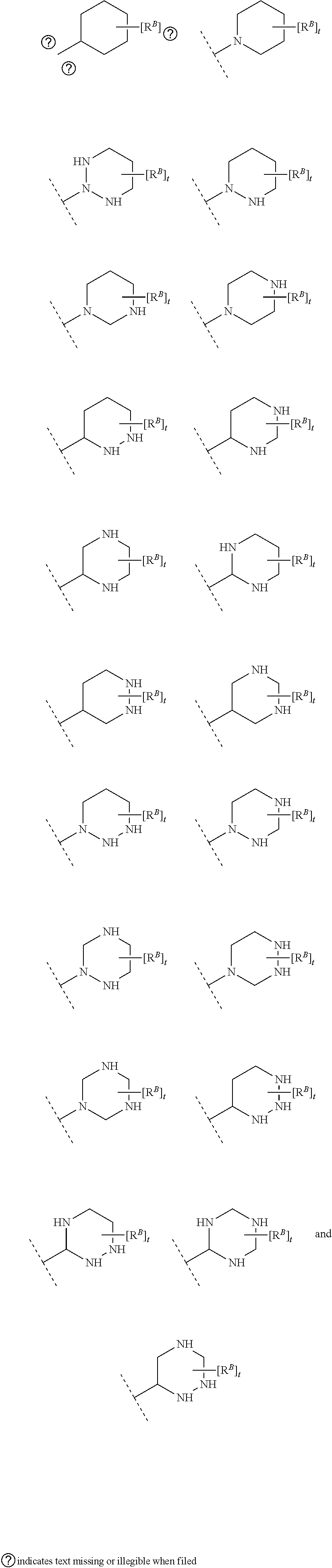

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R_{B} substituent(s) can be covalently attached to any ring atom(s) of A¹ in place of hydrogen, provided the maximum valency of the ring atom(s) to which R^{B} is attached is not exceeded;
t is 0, 1, 2, 3, 4, 5, 6, 7, 8, 9, 10 or 11.

A particular embodiment of the present invention relates to compounds of formula (I), wherein t is 0.

A particular embodiment of the present invention relates to compounds of formula (I), wherein t is 1 or 2.

A particular embodiment of the present invention relates to compounds of formula (I), wherein A¹ is selected from the group of

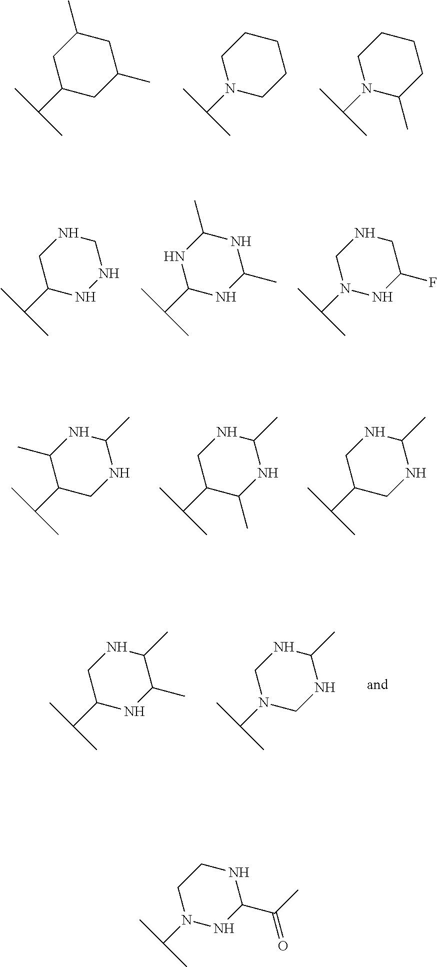

A particular embodiment of the present invention relates to compounds of formula (I), wherein A¹ is selected from the group of hydrogen, deuterium, C₁₋₇-alkyl (e.g., methyl, ethyl, propyl, isopropyl, isobutyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, methoxyacetyl, 2-hydroxyacetyl, tert-butyl acetyl, 2-hydroxypropanal, formamidyl, sulfonyl, methylsulfonyl, dimethylsulfonyl, ethylsulfonyl, 1-methylcarboxamido, N-ethylcarboxamido, N,N-dimethylamino, 1,1,1-trifluoro-2-hydroxybutyl, 5,5,5-trifluoro-2-oxopentyl, or hydroxycyclobutyl, particularly, tert-butyl acetyl, methoxyacetyl, isopropyl, isobutyl, 1,1,1-trifluoro-2-hydroxybutyl, 5,5,5-trifluoro-2-oxopentyl, dimethylsulfonyl, or hydroxycyclobutyl.

A particular embodiment of the present invention relates to compounds of formula (I),

wherein A² is

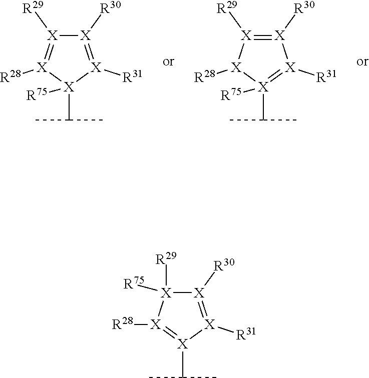

wherein
each X is independently C, N, O or S;
R²⁸, R²⁹, R³⁰, R³¹, and R⁷⁵ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R²⁸, R²⁹, R³⁰, R³¹, or R⁷⁵) attached to that particular X.

A particular embodiment of the present invention relates to compounds of formula (I), wherein R²⁸ is hydrogen or C₁₋₇-alkyl, particularly hydrogen or methyl.

A particular embodiment of the present invention relates to compounds of formula (I), wherein R²⁹ is hydrogen or C₁₋₇-alkyl, particularly hydrogen or methyl.

A particular embodiment of the present invention relates to compounds of formula (I), wherein R³⁰ is hydrogen or C₁₋₇-alkyl, particularly hydrogen or methyl.

A particular embodiment of the present invention relates to compounds of formula (I), wherein R³¹ is hydrogen or C₁₋₇-alkyl, particularly hydrogen or methyl.

A particular embodiment of the present invention relates to compounds of formula (I), wherein R⁷⁵ is hydrogen or C₁₋₇-alkyl, particularly hydrogen or methyl.

A particular embodiment of the present invention relates to compounds of formula (I), wherein one of R²⁸, R²⁹, R³⁰, R³¹, or R⁷⁵ is other than hydrogen.

A particular embodiment of the present invention relates to compounds of formula (I), wherein at least one of R²⁸, R²⁹, R³⁰, R³¹, or R⁷⁵ is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A² is selected from the group of:

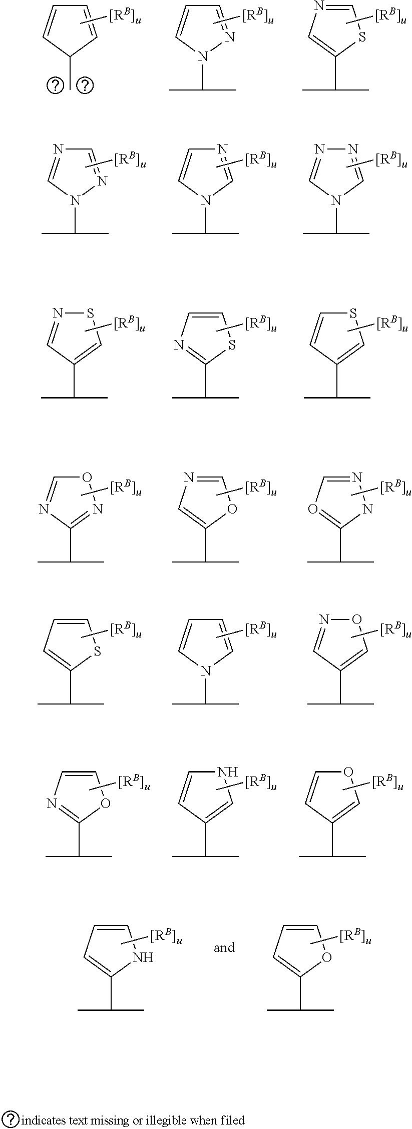

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A² in place of hydrogen, provided the maximum valency of the ring atom(s) to which R^{B} is attached is not exceeded;
u is 0, 1, 2, 3, or 4.

A particular embodiment of the present invention relates to compounds of formula (I), wherein u is 0.

A particular embodiment of the present invention relates to compounds of formula (I), wherein u is 1 or 2.

A particular embodiment of the present invention relates to compounds of formula (I), wherein A² is selected from the group of

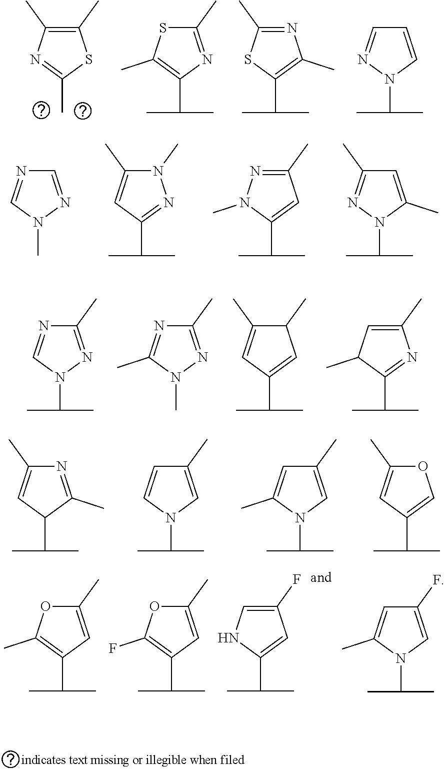

A particular embodiment of the present invention relates to compounds of formula (I),
wherein A² is

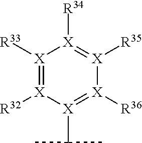

wherein
each X is independently C, N, O or S;
R³², R³³, R³⁴, R³⁵, and R³⁶ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R³², R³³, R³⁴, R³⁵, or R³⁶) attached to that X.

A particular embodiment of the present invention relates to compounds of formula (I), wherein R³² is hydrogen or C₁₋₇-alkyl, particularly hydrogen or methyl.

A particular embodiment of the present invention relates to compounds of formula (I), wherein R³³ is hydrogen or C₁₋₇-alkyl, particularly hydrogen or methyl.

A particular embodiment of the present invention relates to compounds of formula (I), wherein R³¹ is hydrogen or C₁₋₇-alkyl, particularly hydrogen or methyl.

A particular embodiment of the present invention relates to compounds of formula (I), wherein R³⁵ is hydrogen or C₁₋₇-alkyl, particularly hydrogen or methyl.

A particular embodiment of the present invention relates to compounds of formula (I), wherein R³⁶ is hydrogen or C₁₋₇-alkyl, particularly hydrogen or methyl.

A particular embodiment of the present invention relates to compounds of formula (I), wherein one of R³², R³³, R³⁴, R³⁵, or R³⁶, is other than hydrogen.

A particular embodiment of the present invention relates to compounds of formula (I), wherein at least one of R³², R³³, R³⁴, R³⁵, or R³⁶, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A² is selected from the group of

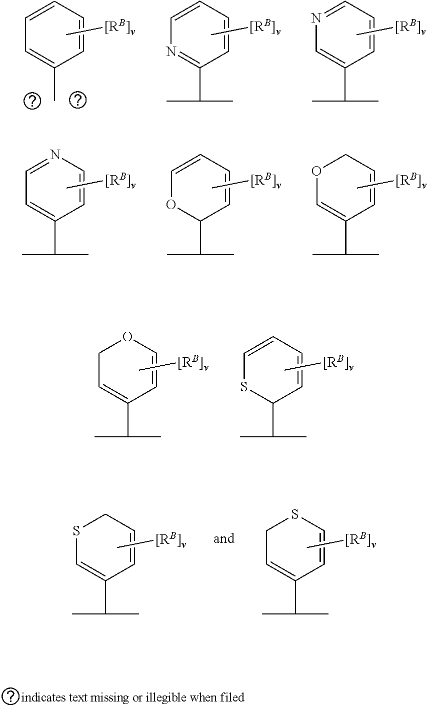

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A² in place of hydrogen, provided the maximum valency of the ring atom(s) to which R^{B} is attached is not exceeded;
v is 0, 1, 2, 3, 4, 5 or 6.

A particular embodiment of the present invention relates to compounds of formula (I), wherein v is 0.

A particular embodiment of the present invention relates to compounds of formula (I), wherein v is 1 or 2.

A particular embodiment of the present invention relates to compounds of formula (I), wherein A² is selected from the group of

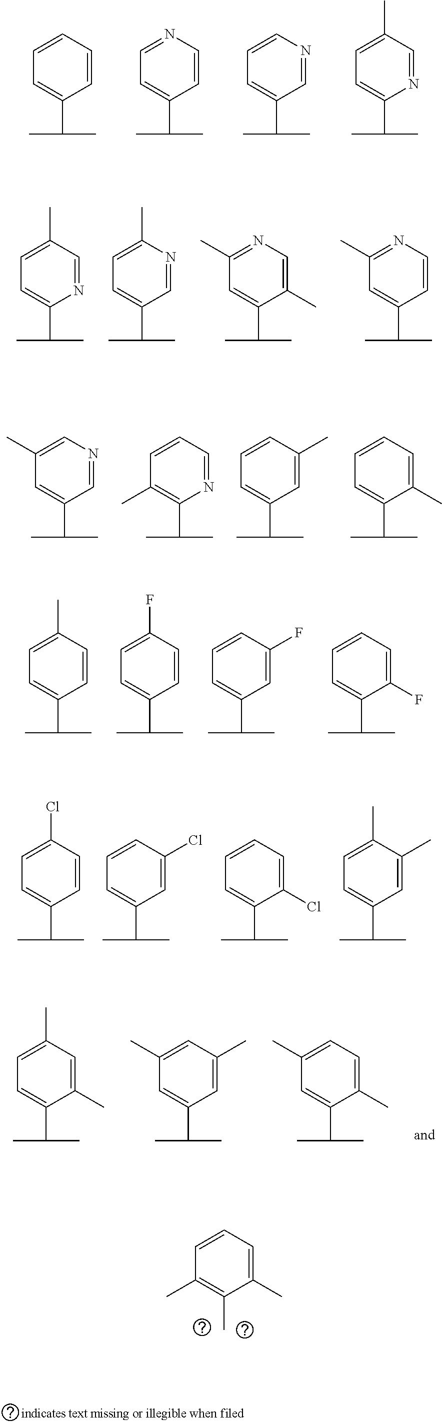

A particular embodiment of the present invention relates to compounds of formula (I),

wherein A² is

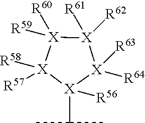

wherein
each X is independently C, N, O or S;
R⁵⁶, R⁵⁷, R⁵⁸, R⁵⁹, R⁶⁰, R⁶¹, R⁶², R⁶³, and R⁶⁴ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R⁵⁶, R⁵⁷, R⁵⁸, R⁵⁹, R⁶⁰, R⁶¹, R⁶², R⁶³, or R⁶⁴) attached to that X.

A particular embodiment of the present invention relates to compounds of formula (I), wherein R⁵⁶ is hydrogen or C₁₋₇-alkyl, particularly hydrogen or methyl.

A particular embodiment of the present invention relates to compounds of formula (I), wherein R⁵⁷ is hydrogen or C₁₋₇-alkyl, particularly hydrogen or methyl.

A particular embodiment of the present invention relates to compounds of formula (I), wherein R⁵⁸ is hydrogen or C₁₋₇-alkyl, particularly hydrogen or methyl.

A particular embodiment of the present invention relates to compounds of formula (I), wherein R⁵⁹ is hydrogen or C₁₋₇-alkyl, particularly hydrogen or methyl.

A particular embodiment of the present invention relates to compounds of formula (I), wherein R⁶⁰ is hydrogen or C₁₋₇-alkyl, particularly hydrogen or methyl.

A particular embodiment of the present invention relates to compounds of formula (I), wherein R⁶¹ is hydrogen or C₁₋₇-alkyl, particularly hydrogen or methyl.

A particular embodiment of the present invention relates to compounds of formula (I), wherein R⁶² is hydrogen or C₁₋₇-alkyl, particularly hydrogen or methyl.

A particular embodiment of the present invention relates to compounds of formula (I), wherein R⁶³ is hydrogen or C₁₋₇-alkyl, particularly hydrogen or methyl.

A particular embodiment of the present invention relates to compounds of formula (I), wherein R⁶⁴ is hydrogen or C₁₋₇-alkyl, particularly hydrogen or methyl.

A particular embodiment of the present invention relates to compounds of formula (I), wherein one of R⁵⁶, R⁵⁷, R⁵⁸, R⁵⁹, R⁶⁰, R⁶¹, R⁶², R⁶³, or R⁶⁴, is other than hydrogen.

A particular embodiment of the present invention relates to compounds of formula (I), wherein at least one of R⁵⁶, R⁵⁷, R⁵⁸, R⁵⁹, R⁶⁰, R⁶¹, R⁶², R⁶³, or R⁶⁴, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino.

A particular embodiment of the present invention relates to compounds of formula (I), wherein A² is selected from the group of

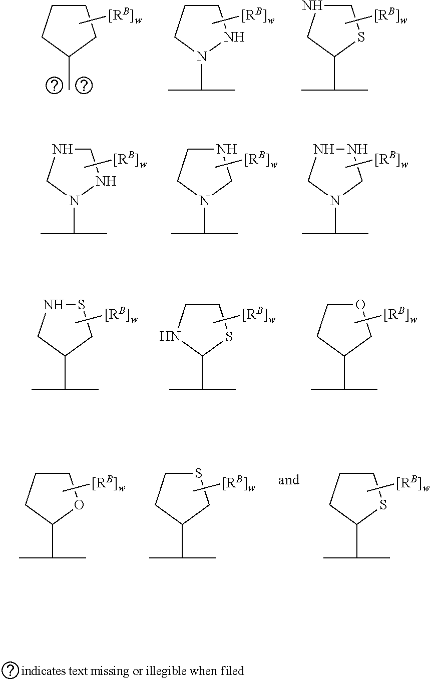

wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A² in place of hydrogen, provided the maximum valency of the ring atom(s) to which R^{B} is attached is not exceeded;
w is 0, 1, 2, 3, 4, 5, 6, 7, 8, or 9.

A particular embodiment of the present invention relates to compounds of formula (I), wherein w is 0.

A particular embodiment of the present invention relates to compounds of formula (I), wherein w is 1 or 2.

A particular embodiment of the present invention relates to compounds of formula (I), wherein A² is selected from the group of

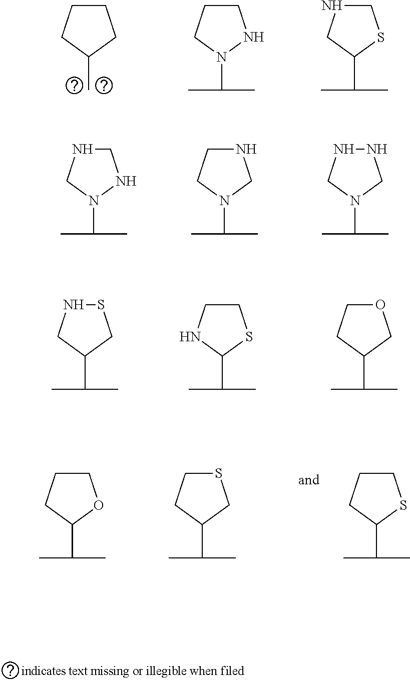

A particular embodiment of the present invention relates to compounds of formula (I),

wherein A² is

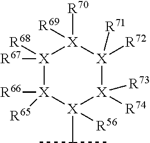

wherein
each X is independently C, N, O or S;
R⁵⁶, R⁶⁵, R⁶⁶, R⁶⁷, R⁶⁸, R⁶⁹, R⁷⁰, R⁷¹, R⁷², R⁷³, and R⁷⁴ are each independently hydrogen, deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
with the proviso that if the maximum valency of a particular X is saturated by the formation of covalent bonds with other ring atoms, then there is no substituent (i.e., R⁵⁶, R⁶⁵, R⁶⁶, R⁶⁷, R⁶⁸, R⁶⁹, R⁷⁰, R⁷¹, R⁷², R⁷³, or R⁷⁴) attached to that X.

A particular embodiment of the present invention relates to compounds of formula (I), wherein R⁶⁵ is hydrogen or C₁₋₇-alkyl, particularly hydrogen or methyl.

A particular embodiment of the present invention relates to compounds of formula (I), wherein R⁶⁶ is hydrogen or C₁₋₇-alkyl, particularly hydrogen or methyl.

A particular embodiment of the present invention relates to compounds of formula (I), wherein R⁶⁷ is hydrogen or C₁₋₇-alkyl, particularly hydrogen or methyl.

A particular embodiment of the present invention relates to compounds of formula (I), wherein R⁶⁸ is hydrogen or C₁₋₇-alkyl, particularly hydrogen or methyl.

A particular embodiment of the present invention relates to compounds of formula (I), wherein R⁶⁹ is hydrogen or C₁₋₇-alkyl, particularly hydrogen or methyl.

A particular embodiment of the present invention relates to compounds of formula (I), wherein R⁷⁰ is hydrogen or C₁₋₇-alkyl, particularly hydrogen or methyl.

A particular embodiment of the present invention relates to compounds of formula (I), wherein R⁷¹ is hydrogen or C₁₋₇-alkyl, particularly hydrogen or methyl.

A particular embodiment of the present invention relates to compounds of formula (I), wherein R⁷² is hydrogen or C₁₋₇-alkyl, particularly hydrogen or methyl.

A particular embodiment of the present invention relates to compounds of formula (I), wherein R⁷³ is hydrogen or C₁₋₇-alkyl, particularly hydrogen or methyl.

A particular embodiment of the present invention relates to compounds of formula (I), wherein R⁷⁴ is hydrogen or C₁₋₇-alkyl, particularly hydrogen or methyl.

A particular embodiment of the present invention relates to compounds of formula (I), wherein one of R⁵⁶, R⁶⁵, R⁶⁶, R⁶⁷, R⁶⁸, R⁶⁹, R⁷⁰, R⁷¹, R⁷², R⁷³, or R⁷⁴, is other than hydrogen.

A particular embodiment of the present invention relates to compounds of formula (I), wherein at least one of R⁶, R⁶⁵, R⁶⁶, R⁶⁷, R⁶⁸, R⁶⁹, R⁷⁰, R⁷¹, R⁷², R⁷³, or R⁷⁴, is deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylanmino.

A particular embodiment of the present invention relates to compounds of formula (I), wherein A² is selected from the group of

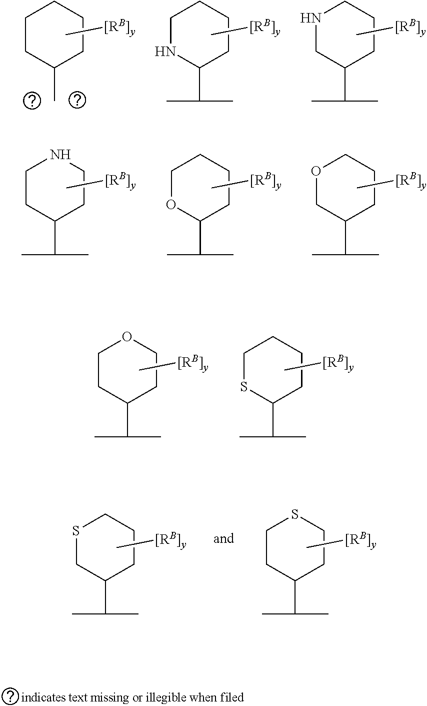

wherein
R^{B} represents non-hydrogen substituent(s), wherein each R^{B} is independently deuterium, C₁₋₇-alkyl (e.g., methyl, ethyl, propyl, isopropyl, etc.), C₃₋₉-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A² in place of hydrogen, provided the maximum valency of the ring atom(s) to which R^{B} is attached is not exceeded;
y is 0, 1, 2, 3, 4, 5, 6, 7, 8, 9, 10 or 11.

A particular embodiment of the present invention relates to compounds of formula (I), wherein y is 0.

A particular embodiment of the present invention relates to compounds of formula (I), wherein y is 1 or 2.

A particular embodiment of the present invention relates to compounds of formula (I), wherein A² is selected from the group of

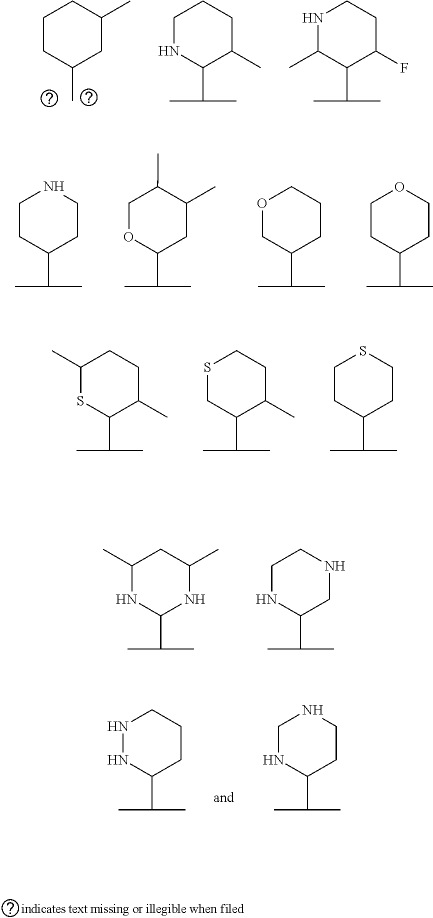

A particular embodiment of the present invention relates to compounds of formula (I), wherein A² is hydrogen.

A particular embodiment of the present invention relates to compounds of formula (I), wherein A² is tert-butyl.

Particular compounds of formula (I) of the present invention are those selected from the group consisting of: 6-(2,4-dimethyl-1,3-thiazol-5-yl)-2-[(1-pyrido[2,3-d]pyrimidin-4-ylpiperidin-4-yl)methyl]pyridazin-3-one; 2-[[1-(7-fluoroquinazolin-4-yl)piperidin-4-yl]methyl]-6-pyrazol-1-ylpyridazin-3-one; 6-(2,4-dimethyl-1,3-thiazol-5-yl)-2-[[1-(6-methylpyrimidin-4-yl)piperidin-4-yl]methyl]pyridazin-3-one; 6-(2,4-dimethyl-1,3-thiazol-5-yl)-2-[[1-(2-methylpyrido[3,4-d]pyrimidin-4-yl)piperidin-4-yl]methyl]pyridazin-3-one; 6-(2,4-dimethyl-1,3-thiazol-5-yl)-2-[[1-(7H-purin-6-yl)piperidin-4-yl]methyl]pyridazin-3-one; 2-[[1-(6-fluoroquinazolin-4-yl)piperidin-4-yl]methyl]-6-(1,2,4-triazol-1-yl)pyridazin-3-one; 2-[[1-(1,6-dimethylpyrazolo[3,4-d]pyrimidin-4-yl)azetidin-3-yl]methyl]-6-pyridin-4-ylpyridazin-3-one; 2-[1-(1,6-dimethylpyrazolo[3,4-d]pyrimidin-4-yl)azetidin-3-yl]-6-pyridin-4-ylpyridazin-3-one; 6-pyrazol-1-yl-2-[[1-[3-(trifluoromethyl)-[1,2,4]triazolo[4,3-b]pyridazin-6-yl]piperidin-4-yl]methyl]pyridazin-3-one; 6-(1,2,4-triazol-1-yl)-2-[[1-[3-(trifluoromethyl)-[1,2,4]triazolo[4,3-b]pyridazin-6-yl]piperidin-4-yl]methyl]pyridazin-3-one; 2-[[1-(7H-purin-6-yl)azetidin-3-yl]methyl]-6-pyridin-4-ylpyridazin-3-one; 2-[[1-(6-methylpyrazin-2-yl)piperidin-4-yl]methyl]-6-(1,2,4-triazol-1-yl)pyridazin-3-one; 6-(3,5-dimethylpyrazol-1-yl)-2-[[1-(3-methyl-[1,2,4]triazolo[4,3-b]pyridazin-6-yl)azetidin-3-yl]methyl]pyridazin-3-one; 6-(3,5-dimethylpyrazol-1-yl)-2-[l-(3-methylimidazo[4,5-b]pyridin-2-yl)piperidin-4-yl]pyridazin-3-one; 2-((1-(5H-pyrazolo[3,4-d]pyrimidin-4-yl)piperidin-4-yl)methyl)-6-(2,4-dimethylthiazol-5-yl)pyridazin-3(2H)-one; 6-(2,4-dimethyl-1,3-thiazol-5-yl)-2-[(1-pyrazolo[1,5-a]pyrimidin-5-ylpiperidin-4-yl)methyl]pyridazin-3-one; 6-(2,4-dimethyl-1,3-thiazol-5-yl)-2-[[1-(2-methylpyrimidin-4-yl)piperidin-4-yl]methyl]pyridazin-3-one; 6-(2,4-dimethyl-1,3-thiazol-5-yl)-2-[[1-(2-propan-2-ylpyrimidin-4-yl)piperidin-4-yl]methyl]pyridazin-3-one; 6-(2,4-dimethyl-1,3-thiazol-5-yl)-2-[[1-(9-methylpurin-6-yl)piperidin-4-yl]methyl]pyridazin-3-one; 6-(2,4-dimethylthiazol-5-yl)-2-((1-(6-methyl-3H-pyrrolo[3,2-d]pyrimidin-4-yl)piperidin-4-yl)methyl)pyridazin-3(2H)-one; 6-(2,4-dimethyl-1,3-thiazol-5-yl)-2-[[1-(6-ethyl-5-fluoropyrimidin-4-yl)piperidin-4-yl]methyl]pyridazin-3-one; 6-(2,4-dimethyl-1,3-thiazol-5-yl)-2-[(1-thieno[2,3-d]pyrimidin-4-ylpiperidin-4-yl)methyl]pyridazin-3-one; 6-(2,4-dimethylthiazol-5-yl)-2-((1-(imidazo[1,2-b]pyridazin-6-yl)piperidin-4-yl)methyl)pyridazin-3(2H)-one; 2-[4-[[3-(2,4-dimethyl-1,3-thiazol-5-yl)-6-oxopyridazin-1-yl]methyl]piperidin-1-yl]pyridine-4-carbonitrile; 6-[4-[[3-(2,4-dimethyl-1,3-thiazol-5-yl)-6-oxopyridazin-1-yl]methyl]piperidin-1-yl]pyridine-3-carbonitrile; 6-(2,4-dimethyl-1,3-thiazol-5-yl)-2-[(1-thieno[3,2-d]pyrimidin-4-ylpiperidin-4-yl)methyl]pyridazin-3-one; 2-[4-[[3-(2,4-dimethyl-1,3-thiazol-5-yl)-6-oxopyridazin-1-yl]methyl]piperidin-1-yl]pyridine-3-carbonitrile; 6-(2,4-dimethylthiazol-5-yl)-2-((1-(6-(trifluoromethyl)pyridin-2-yl)piperidin-4-yl)methyl)pyridazin-3(2H)-one; 6-(2,4-dimethylthiazol-5-yl)-2-((1-(4-(trifluoromethyl)pyridin-2-yl)piperidin-4-yl)methyl)pyridazin-3(2H)-one; 6-(2,4-dimethyl-1,3-thiazol-5-yl)-2-[[1-(5-methylpyrimidin-2-yl)piperidin-4-yl]methyl]pyridazin-3-one; 6-(2,4-dimethyl-1,3-thiazol-5-yl)-2-[(1-pyrimidin-2-ylpiperidin-4-yl)methyl]pyridazin-3-one; 6-(2,4-dimethylthiazol-5-yl)-2-((1-(5-(trifluoromethyl)pyridin-2-yl)piperidin-4-yl)methyl)pyridazin-3(2H)-one; 6-(2,4-dimethyl-1,3-thiazol-5-yl)-2-[[1-(3-methyl-[1,2,4]triazolo[4,3-b]pyridazin-6-yl)piperidin-4-yl]methyl]pyridazin-3-one; 6-(2,4-dimethyl-1,3-thiazol-5-yl)-2-[(1-pyrazolo[1,5-a]pyrazin-4-ylpiperidin-4-yl)methyl]pyridazin-3-one; 6-(2,4-dimethylthiazol-5-yl)-2-((1-(3-(trifluoromethyl)pyridin-2-yl)piperidin-4-yl)methyl)pyridazin-3(2H)-one; 2-[[1-(5-chloropyrimidin-2-yl)piperidin-4-yl]methyl]-6-(2,4-dimethyl-1,3-thiazol-5-yl)pyridazin-3-one; 6-(2,4-dimethyl-1,3-thiazol-5-yl)-2-[[1-(4-methylpyrimidin-2-yl)piperidin-4-yl]methyl]pyridazin-3-one; 2-[[1-(4,6-dimethylpyrimidin-2-yl)piperidin-4-yl]methyl]-6-(2,4-dimethyl-1,3-thiazol-5-yl)pyridazin-3-one; 6-(2,4-dimethylthiazol-5-yl)-2-((1-(5,6,7,8-tetrahydroquinazolin-4-yl)piperidin-4-yl)methyl)pyridazin-3(2H)-one; 6-(2,4-dimethyl-1,3-thiazol-5-yl)-2-[(1-quinoxalin-2-ylpiperidin-4-yl)methyl]pyridazin-3-one; 6-(2,4-dimethyl-1,3-thiazol-5-yl)-2-[[1-(5-fluoropyrimidin-2-yl)piperidin-4-yl]methyl]pyridazin-3-one; 5-[4-[[3-(2,4-dimethyl-1,3-thiazol-5-yl)-6-oxopyridazin-1-yl]methyl]piperidin-1-yl]pyridine-2-carbonitrile; 6-(2,4-dimethyl-1,3-thiazol-5-yl)-2-[[1-(2-methylpyrazolo[1,5-a]pyrazin-4-yl)piperidin-4-yl]methyl]pyridazin-3-one; 2-[4-[[3-(2,4-dimethyl-1,3-thiazol-5-yl)-6-oxopyridazin-1-yl]methyl]piperidin-1-yl]pyrimidine-4-carbonitrile; 6-(2,4-dimethyl-1,3-thiazol-5-yl)-2-[[1-(4-methoxypyrimidin-2-yl)piperidin-4-yl]methyl]pyridazin-3-one; 6-(2,4-dimethylthiazol-5-yl)-2-(piperidin-4-ylmethyl)pyridazin-3(2H)-one; tert-butyl 4-((3-(2,4-dimethylthiazol-5-yl)-6-oxopyridazin-1(6H)-yl)methyl)piperidine-1-carboxylate; 3-(4-((3-(2,4-dimethylthiazol-5-yl)-6-oxopyridazin-1(6H)-yl)methyl)piperidin-1-yl)pyrazine-2-carbonitrile; 4-[[4-[[3-(2,4-dimethyl-1,3-thiazol-5-yl)-6-oxopyridazin-1-yl]methyl]piperidin-1-yl]methyl]benzonitrile; 6-(2,4-dimethylthiazol-5-yl)-2-((1-(2-fluorobenzyl)piperidin-4-yl)methyl)pyridazin-3(2H)-one; 6-(2,4-dimethylthiazol-5-yl)-2-((1-(4-fluorobenzyl)piperidin-4-yl)methyl)pyridazin-3(2H)-one; 6-(2,4-dimethylthiazol-5-yl)-2-((1-(3-fluorobenzyl)piperidin-4-yl)methyl)pyridazin-3(2H)-one; 2-((1-(2-chlorobenzyl)piperidin-4-yl)methyl)-6-(2,4-dimethylthiazol-5-yl)pyridazin-3(2H)-one; 2-((1-(4-chlorobenzyl)piperidin-4-yl)methyl)-6-(2,4-dimethylthiazol-5-yl)pyridazin-3(2H)-one; 2-((1-(3-chlorobenzyl)piperidin-4-yl)methyl)-6-(2,4-dimethylthiazol-5-yl)pyridazin-3(2H)-one; 6-(2,4-dimethyl-1,3-thiazol-5-yl)-2-[[1-(3-methylquinoxalin-2-yl)piperidin-4-yl]methyl]pyridazin-3-one; 6-(2,4-dimethyl-1,3-thiazol-5-yl)-2-[[1-(2-methyl-6,7-dihydro-5H-cyclopenta[d]pyrimidin-4-yl)piperidin-4-yl]methyl]pyridazin-3-one; 2-((1-(3,5-difluorobenzyl)piperidin-4-yl)methyl)-6-(2,4-dimethylthiazol-5-yl)pyridazin-3(2H)-one; 2-[[1-[(3,4-difluorophenyl)methyl]piperidin-4-yl]methyl]-6-(2,4-dimethyl-1,3-thiazol-5-yl)pyridazin-3-one; 2-[[1-[(2-chloro-6-fluorophenyl)methyl]piperidin-4-yl]methyl]-6-(2,4-dimethyl-1,3-thiazol-5-yl)pyridazin-3-one; 2-[[1-[(2,5-difluorophenyl)methyl]piperidin-4-yl]methyl]-6-(2,4-dimethyl-1,3-thiazol-5-yl)pyridazin-3-one; 2-[[1-[(2,4-difluorophenyl)methyl]piperidin-4-yl]methyl]-6-(2,4-dimethyl-1,3-thiazol-5-yl)pyridazin-3-one; 2-[[1-(6-fluoroquinazolin-4-yl)piperidin-4-yl]methyl]-6-pyrazol-1-ylpyridazin-3-one; 6-(3,5-dimethylpyrazol-1-yl)-2-[[1-(7-fluoroquinazolin-4-yl)azetidin-3-yl]methyl]pyridazin-3-one; 6-(3,5-dimethylpyrazol-1-yl)-2-[[1-(6-fluoroquinazolin-4-yl)azetidin-3-yl]methyl]pyridazin-3-one; 6-(3,5-dimethylpyrazol-1-yl)-2-(1-pyrido[3,4-d]pyrimidin-4-ylpiperidin-4-yl)pyridazin-3-one; 6-(3,5-dimethylpyrazol-1-yl)-2-(1-quinolin-4-ylpiperidin-4-yl)pyridazin-3-one; 6-(3,5-dimethylpyrazol-1-yl)-2-(1-quinolin-2-ylpiperidin-4-yl)pyridazin-3-one; 2-[[1-(7-fluoroquinazolin-4-yl)piperidin-4-yl]methyl]-6-(1,2,4-triazol-1-yl)pyridazin-3-one; 6-(3,5-dimethylpyrazol-1-yl)-2-[1-(2,6-dimethylpyrimidin-4-yl)piperidin-4-yl]pyridazin-3-one; 2-[1-(2-cyclopropylpyrimidin-4-yl)piperidin-4-yl]-6-(3,5-dimethylpyrazol-1-yl)pyridazin-3-one; 6-(3,5-dimethylpyrazol-1-yl)-2-[1-(1-methyl-6-oxopyrimidin-4-yl)piperidin-4-yl]pyridazin-3-one; 2-[4-[3-(3,5-dimethylpyrazol-1-yl)-6-oxopyridazin-1-yl]piperidin-1-yl]quinoline-3-carbonitrile; 6-(3,5-dimethylpyrazol-1-yl)-2-[1-(5,6-dimethylpyrimidin-4-yl)piperidin-4-yl]pyridazin-3-one; 2-[[1-(6-methyl-5H-pyrrolo[3,2-d]pyrimidin-4-yl)piperidin-4-yl]methyl]-6-pyrazol-1-ylpyridazin-3-one; 2-[4-[3-(3,5-dimethylpyrazol-1-yl)-6-oxopyridazin-1-yl]piperidin-1-yl]quinoline-4-carbonitrile; 6-[4-[3-(3,5-dimethylpyrazol-1-yl)-6-oxopyridazin-1-yl]piperidin-1-yl]pyridine-2-carbonitrile; 6-[4-[3-(3,5-dimethylpyrazol-1-yl)-6-oxopyridazin-1-yl]piperidin-1-yl]-3-methyl-1H-pyrimidine-2,4-dione; 6-(3,5-dimethylpyrazol-1-yl)-2-(1-thieno[3,2-d]pyrimidin-4-ylpiperidin-4-yl)pyridazin-3-one; 6-(3,5-dimethylpyrazol-1-yl)-2-(1-thieno[2,3-d]pyrimidin-4-ylpiperidin-4-yl)pyridazin-3-one; 2-[[1-(7H-purin-6-yl)piperidin-4-yl]methyl]-6-pyrazol-1-ylpyridazin-3-one; 6-(3,5-dimethylpyrazol-1-yl)-2-[[1-(7H-pyrrolo[2,3-d]pyrimidin-4-yl)azetidin-3-yl]methyl]pyridazin-3-one; 6-(3,5-dimethylpyrazol-1-yl)-2-[1-(3-methylquinoxalin-2-yl)piperidin-4-yl]pyridazin-3-one; 2-[1-(6-cyclobutylpyrimidin-4-yl)piperidin-4-yl]-6-(3,5-dimethylpyrazol-1-yl)pyridazin-3-one; 6-(3,5-dimethylpyrazol-1-yl)-2-[1-(9-methylpurin-6-yl)piperidin-4-yl]pyridazin-3-one; 6-(3,5-dimethylpyrazol-1-yl)-2-[1-[2-methyl-6-(trifluoromethyl)pyrimidin-4-yl]piperidin-4-yl]pyridazin-3-one; 2-[4-[(6-oxo-3-pyrazol-1-ylpyridazin-1-yl)methyl]piperidin-1-yl]pyrido[1,2-a]pyrimidin-4-one; 6-(3,5-dimethylpyrazol-1-yl)-2-[[1-(5-fluoropyrimidin-4-yl)azetidin-3-yl]methyl]pyridazin-3-one; 6-pyrazol-1-yl-2-[(1-thieno[3,2-d]pyrimidin-4-ylpiperidin-4-yl)methyl]pyridazin-3-one; 6-pyrazol-1-yl-2-[(1-thieno[2,3-d]pyrimidin-4-ylpiperidin-4-yl)methyl]pyridazin-3-one; 2-[[1-(1-methylpyrazolo[3,4-d]pyrimidin-4-yl)piperidin-4-yl]methyl]-6-pyrazol-1-ylpyridazin-3-one; 6-pyrazol-1-yl-2-[[1-[6-(trifluoromethyl)pyrimidin-4-yl]piperidin-4-yl]methyl]pyridazin-3-one; 6-(3,5-dimethylpyrazol-1-yl)-2-[(1-thieno[3,2-d]pyrimidin-4-ylazetidin-3-yl)methyl]pyridazin-3-one; 6-(3,5-dimethylpyrazol-1-yl)-2-[[1-(7H-purin-6-yl)azetidin-3-yl]methyl]pyridazin-3-one; 6-(3,5-dimethylpyrazol-1-yl)-2-[[1-[6-(trifluoromethyl)pyrimidin-4-yl]azetidin-3-yl]methyl]pyridazin-3-one; 6-(3,5-dimethylpyrazol-1-yl)-2-[1-(4,6-dimethylpyrimidin-2-yl)piperidin-4-yl]pyridazin-3-one; 6-(3,5-dimethylpyrazol-1-yl)-2-[(1-thieno[2,3-d]pyrimidin-4-ylazetidin-3-yl)methyl]pyridazin-3-one; 6-(3,5-dimethylpyrazol-1-yl)-2-[1-(5,6-dimethyl-[1,2,4]triazolo[1,5-a]pyrimidin-7-yl)piperidin-4-yl]pyridazin-3-one; 6-(3,5-dimethylpyrazol-1-yl)-2-[[1-(5-methylpyrazolo[1,5-a]pyrimidin-7-yl)azetidin-3-yl]methyl]pyridazin-3-one; 2-[[1-(1,6-dimethylpyrazolo[3,4-d]pyrimidin-4-yl)piperidin-4-yl]methyl]-6-pyrazol-1-ylpyridazin-3-one; 2-[[1-(2,5-dimethylpyrazolo[1,5-a]pyrimidin-7-yl)azetidin-3-yl]methyl]-6-(3,5-dimethylpyrazol-1-yl)pyridazin-3-one; 2-[4-[(6-oxo-3-pyrazol-1-ylpyridazin-1-yl)methyl]piperidin-1-yl]pyridine-3-carbonitrile; 2-[[1-(7H-pyrrolo[2,3-d]pyrimidin-4-yl)piperidin-4-yl]methyl]-6-(1,2,4-triazol-1-yl)pyridazin-3-one; 6-(3,5-dimethylpyrazol-1-yl)-2-[1-(3-methyl-[1,2,4]triazolo[4,3-a]pyrazin-8-yl)piperidin-4-yl]pyridazin-3-one; 6-(3,5-dimethylpyrazol-1-yl)-2-[1-(4-methoxypyrimidin-2-yl)piperidin-4-yl]pyridazin-3-one; 2-[[1-(3-chloropyridin-4-yl)piperidin-4-yl]methyl]-6-pyrazol-1-ylpyridazin-3-one; 3-[3-[[3-(3,5-dimethylpyrazol-1-yl)-6-oxopyridazin-1-yl]methyl]azetidin-1-yl]pyrazine-2-carbonitrile; 6-(3,5-dimethylpyrazol-1-yl)-2-[1-(1-methylbenzimidazol-2-yl)piperidin-4-yl]pyridazin-3-one; 2-[4-[3-(3,5-dimethylpyrazol-1-yl)-6-oxopyridazin-1-yl]piperidin-1-yl]pyridine-4-carbonitrile; 2-[3-[[3-(3,5-dimethylpyrazol-1-yl)-6-oxopyridazin-1-yl]methyl]azetidin-1-yl]pyrido[1,2-a]pyrimidin-4-one; 6-(3,5-dimethylpyrazol-1-yl)-2-[1-(2-methylpyrazolo[1,5-a]pyrazin-4-yl)piperidin-4-yl]pyridazin-3-one; 6-(3,5-dimethylpyrazol-1-yl)-2-[1-(5-fluoropyrimidin-2-yl)piperidin-4-yl]pyridazin-3-one; 6-(3,5-dimethylpyrazol-1-yl)-2-[1-(5-ethylpyrimidin-2-yl)piperidin-4-yl]pyridazin-3-one; 6-(3,5-dimethylpyrazol-1-yl)-2-[1-(2-fluorophenyl)sulfonylpiperidin-4-yl]pyridazin-3-one; N-(2-chlorophenyl)-4-[3-(3,5-dimethylpyrazol-1-yl)-6-oxopyridazin-1-yl]piperidine-1-carboxamide; N-(2,6-difluorophenyl)-4-[3-(3,5-dimethylpyrazol-1-yl)-6-oxopyridazin-1-yl]piperidine-1-carboxamide; 6-(3,5-dimethylpyrazol-1-yl)-2-[[1-(1-methylpyrazolo[3,4-d]pyrimidin-4-yl)azetidin-3-yl]methyl]pyridazin-3-one; 2-[3-[[3-(3,5-dimethylpyrazol-1-yl)-6-oxopyridazin-1-yl]methyl]azetidin-1-yl]pyridine-3-carbonitrile; 6-(3,5-dimethylpyrazol-1-yl)-2-[[1-[3-(trifluoromethyl)pyridin-2-yl]azetidin-3-yl]methyl]pyridazin-3-one; 6-(3,5-dimethylpyrazol-1-yl)-2-[[1-(3-methylquinoxalin-2-yl)azetidin-3-yl]methyl]pyridazin-3-one; 2-[1-(5-bromopyrimidin-2-yl)piperidin-4-yl]-6-(3,5-dimethylpyrazol-1-yl)pyridazin-3-one; 4-[3-(3,5-dimethylpyrazol-1-yl)-6-oxopyridazin-1-yl]-N-(2-phenylethyl)piperidine-1-carboxamide; 6-(3,5-dimethylpyrazol-1-yl)-2-[1-[2-(4-fluorophenoxy)acetyl]piperidin-4-yl]pyridazin-3-one; 6-(3,5-dimethylpyrazol-1-yl)-2-[1-(pyridin-3-ylmethyl)piperidin-4-yl]pyridazin-3-one; 6-(3,5-dimethylpyrazol-1-yl)-2-piperidin-4-ylpyridazin-3-one; 6-(3,5-dimethylpyrazol-1-yl)-2-[[1-(5-methyl-[1,2,4]triazolo[1,5-a]pyrimidin-7-yl)azetidin-3-yl]methyl]pyridazin-3-one; 2-[1-(3-cyclopropyl-1,2,4-thiadiazol-5-yl)piperidin-4-yl]-6-(3,5-dimethylpyrazol-1-yl)pyridazin-3-one; 4-[3-(3,5-dimethylpyrazol-1-yl)-6-oxopyridazin-1-yl]-N-[(4-fluorophenyl)methyl]piperidine-1-carboxamide; 2-[1-(1,3-benzoxazol-2-yl)piperidin-4-yl]-6-(3,5-dimethylpyrazol-1-yl)pyridazin-3-one; 6-(3,5-dimethylpyrazol-1-yl)-2-[1-(1,3,4-thiadiazol-2-yl)piperidin-4-yl]pyridazin-3-one; 6-(3,5-dimethylpyrazol-1-yl)-2-[1-(2-pyridin-3-ylacetyl)piperidin-4-yl]pyridazin-3-one; 6-[3-[[3-(3,5-dimethylpyrazol-1-yl)-6-oxopyridazin-1-yl]methyl]azetidin-1-yl]pyridine-3-carbonitrile; 2-[i-[2-(2-chloro-6-fluorophenyl)acetyl]piperidin-4-yl]-6-(3,5-dimethylpyrazol-1-yl)pyridazin-3-one; N-(3,4-dimethylphenyl)-4-[3-(3,5-dimethylpyrazol-1-yl)-6-oxopyridazin-1-yl]piperidine-1-carboxamide; 6-(3,5-dimethylpyrazol-1-yl)-2-[1-(1,3-thiazol-2-yl)piperidin-4-yl]pyridazin-3-one; 6-(3,5-dimethylpyrazol-1-yl)-2-[1-[2-(2-fluorophenoxy)propanoyl]piperidin-4-yl]pyridazin-3-one; 2-[1-(3,4-dimethylphenyl)sulfonylpiperidin-4-yl]-6-(3,5-dimethylpyrazol-1-yl)pyridazin-3-one; 3-[4-[(6-oxo-3-pyrazol-1-ylpyridazin-1-yl)methyl]piperidin-1-yl]pyrazine-2-carbonitrile; 6-[4-[(6-oxo-3-pyrazol-1-ylpyridazin-1-yl)methyl]piperidin-1-yl]pyridine-3-carbonitrile; 6-pyrazol-1-yl-2-[[1-([1,2,4]triazolo[4,3-b]pyridazin-6-yl)piperidin-4-yl]methyl]pyridazin-3-one; 2-[[1-(6-methyl-5H-pyrrolo[3,2-d]pyrimidin-4-yl)piperidin-4-yl]methyl]-6-(1,2,4-triazol-1-yl)pyridazin-3-one; 2-[1-[2-(2-chlorophenyl)acetyl]piperidin-4-yl]-6-(3,5-dimethylpyrazol-1-yl)pyridazin-3-one; 6-(3,5-dimethylpyrazol-1-yl)-2-[1-[2-(1-methylindol-3-yl)acetyl]piperidin-4-yl]pyridazin-3-one; 6-(3,5-dimethylpyrazol-1-yl)-2-[1-(isoquinoline-1-carbonyl)piperidin-4-yl]pyridazin-3-one; 6-(3,5-dimethylpyrazol-1-yl)-2-[1-[(E)-3-phenylprop-2-enyl]piperidin-4-yl]pyridazin-3-one; 6-(3,5-dimethylpyrazol-1-yl)-2-[1-(4,4,4-trifluorobutanoyl)piperidin-4-yl]pyridazin-3-one; 6-(3,5-dimethylpyrazol-1-yl)-2-[1-(2-methoxyacetyl)piperidin-4-yl]pyridazin-3-one; 6-(3,5-dimethylpyrazol-1-yl)-2-[1-(4-fluoro-1,3-benzothiazol-2-yl)piperidin-4-yl]pyridazin-3-one; 6-(3,5-dimethylpyrazol-1-yl)-2-[1-(2-phenylethylsulfonyl)piperidin-4-yl]pyridazin-3-one; 2-[(1-quinolin-4-ylpiperidin-4-yl)methyl]-6-(1,2,4-triazol-1-yl)pyridazin-3-one; 2-[[1-[(2,5-difluorophenyl)methyl]piperidin-4-yl]methyl]-6-pyrazol-1-ylpyridazin-3-one; 2-[[1-(5-fluoropyrimidin-4-yl)piperidin-4-yl]methyl]-6-(1,2,4-triazol-1-yl)pyridazin-3-one; 6-pyrazol-1-yl-2-[[1-[3-(trifluoromethyl)pyridin-2-yl]piperidin-4-yl]methyl]pyridazin-3-one; 2-[[1-(2-methylpyrimidin-4-yl)piperidin-4-yl]methyl]-6-(1,2,4-triazol-1-yl)pyridazin-3-one; 2-[[1-(6-tert-butylpyridazin-3-yl)piperidin-4-yl]methyl]-6-pyrazol-1-ylpyridazin-3-one; 2-[[1-(2-phenylethyl)piperidin-4-yl]methyl]-6-pyrazol-1-ylpyridazin-3-one; 2-[[1-(2-indol-1-ylacetyl)piperidin-4-yl]methyl]-6-pyrazol-1-ylpyridazin-3-one; 2-[[1-(6-methylpyrimidin-4-yl)piperidin-4-yl]methyl]-6-(1,2,4-triazol-1-yl)pyridazin-3-one; 2-[[1-(6,7-dihydro-5H-cyclopenta[d]pyrimidin-4-yl)piperidin-4-yl]methyl]-6-pyrazol-1-ylpyridazin-3-one; 2-[[1-[6-(dimethylamino)pyrimidin-4-yl]piperidin-4-yl]methyl]-6-(1,2,4-triazol-1-yl)pyridazin-3-one; 2-[(1-imidazo[1,2-b]pyridazin-6-ylpiperidin-4-yl)methyl]-6-pyrazol-1-ylpyridazin-3-one; 2-[[1-(5-fluoropyrimidin-2-yl)piperidin-4-yl]methyl]-6-pyrazol-1-ylpyridazin-3-one; 2-[[1-(3-methyl-[1,2,4]triazolo[4,3-b]pyridazin-6-yl)piperidin-4-yl]methyl]-6-pyrazol-1-ylpyridazin-3-one; 2-((1-(3aH-indole-6-carbonyl)piperidin-4-yl)methyl)-6-(1H-pyrazol-1-yl)pyridazin-3(2H)-one; 2-[(1-thieno[3,2-d]pyrimidin-4-ylpiperidin-4-yl)methyl]-6-(1,2,4-triazol-1-yl)pyridazin-3-one; 2-[(1-propan-2-ylpiperidin-4-yl)methyl]-6-pyrazol-1-ylpyridazin-3-one; 2-[[1-(1-phenylethyl)piperidin-4-yl]methyl]-6-pyrazol-1-ylpyridazin-3-one; 2-[4-[[6-oxo-3-(1,2,4-triazol-1-yl)pyridazin-1-yl]methyl]piperidin-1-yl]pyrido[1,2-a]pyrimidin-4-one; 2-[[1-(5-chloropyrimidin-2-yl)piperidin-4-yl]methyl]-6-pyrazol-1-ylpyridazin-3-one; 2-[(1-thieno[2,3-d]pyrimidin-4-ylpiperidin-4-yl)methyl]-6-(1,2,4-triazol-1-yl)pyridazin-3-one; 2-[[1-(2-hydroxycyclohexyl)piperidin-4-yl]methyl]-6-pyrazol-1-ylpyridazin-3-one; 2-[[1-[2-(2-fluorophenyl)acetyl]piperidin-4-yl]methyl]-6-pyrazol-1-ylpyridazin-3-one; 2-[(1-cyclopentylpiperidin-4-yl)methyl]-6-pyrazol-1-ylpyridazin-3-one; 2-[[1-(2-hydroxyethyl)piperidin-4-yl]methyl]-6-pyrazol-1-ylpyridazin-3-one; 2-[[1-[(1-hydroxycyclohexyl)methyl]piperidin-4-yl]methyl]-6-pyrazol-1-ylpyridazin-3-one; 2-[[1-[(1-hydroxycyclopentyl)methyl]piperidin-4-yl]methyl]-6-pyrazol-1-ylpyridazin-3-one; 2-[[1-(5-bromopyrimidin-2-yl)piperidin-4-yl]methyl]-6-pyrazol-1-ylpyridazin-3-one; 2-((1-(5H-indole-4-carbonyl)piperidin-4-yl)methyl)-6-(1H-pyrazol-1-yl)pyridazin-3(2H)-one; 2-[[1-(cyclopentylmethyl)piperidin-4-yl]methyl]-6-pyrazol-1-ylpyridazin-3-one; 2-[[1-(2-piperidin-1-ylethyl)piperidin-4-yl]methyl]-6-pyrazol-1-ylpyridazin-3-one; 6-pyrazol-1-yl-2-[[1-[5-(trifluoromethyl)pyridin-2-yl]piperidin-4-yl]methyl]pyridazin-3-one; 2-[[1-(2-methylpropyl)piperidin-4-yl]methyl]-6-pyrazol-1-ylpyridazin-3-one; 2-[(1-benzylpiperidin-4-yl)methyl]-6-pyrazol-1-ylpyridazin-3-one; 6-pyrazol-1-yl-2-[[1-(quinoline-2-carbonyl)piperidin-4-yl]methyl]pyridazin-3-one; 2-((1-(2H-benzo[d]imidazole-5-carbonyl)piperidin-4-yl)methyl)-6-(1H-pyrazol-1-yl)pyridazin-3(2H)-one; 2-[[1-(2-cyclopropyl-6-methylpyrimidin-4-yl)piperidin-4-yl]methyl]-6-(1,2,4-triazol-1-yl)pyridazin-3-one; 6-pyrazol-1-yl-2-[[1-(2,2,2-trifluoroethyl)piperidin-4-yl]methyl]pyridazin-3-one; 2-[[1-(2-methyl-6,7-dihydro-5H-cyclopenta[d]pyrimidin-4-yl)piperidin-4-yl]methyl]-6-pyrazol-1-ylpyridazin-3-one; 6-pyrazol-1-yl-2-[[1-(3,3,3-trifluoro-2-hydroxypropyl)piperidin-4-yl]methyl]pyridazin-3-one; 2-(piperidin-4-ylmethyl)-6-pyrazol-1-ylpyridazin-3-one; 2-[[1-(oxan-2-ylmethyl)piperidin-4-yl]methyl]-6-pyrazol-1-ylpyridazin-3-one; 2-[[1-(2-fluorobenzoyl)piperidin-4-yl]methyl]-6-pyrazol-1-ylpyridazin-3-one; 2-[[1-[(4-hydroxyoxan-4-yl)methyl]piperidin-4-yl]methyl]-6-pyrazol-1-ylpyridazin-3-one; 2-[[1-(2-hydroxycyclobutyl)piperidin-4-yl]methyl]-6-pyrazol-1-ylpyridazin-3-one; 2-[[1-(7H-purin-6-yl)piperidin-4-yl]methyl]-6-(1,2,4-triazol-1-yl)pyridazin-3-one; 2-[[1-[2-(4-chlorophenyl)acetyl]piperidin-4-yl]methyl]-6-pyrazol-1-ylpyridazin-3-one; 4-[[4-[(6-oxo-3-pyrazol-1-ylpyridazin-1-yl)methyl]piperidin-1-yl]methyl]benzonitrile; 2-[4-[(6-oxo-3-pyrazol-1-ylpyridazin-1-yl)methyl]piperidin-1-yl]-5,6,7,8-tetrahydroquinoline-3-carbonitrile; 2-[[1-(oxan-4-ylmethyl)piperidin-4-yl]methyl]-6-pyrazol-1-ylpyridazin-3-one; 6-pyrazol-1-yl-2-[[1-(quinoxaline-6-carbonyl)piperidin-4-yl]methyl]pyridazin-3-one; 2-[[1-(isoquinoline-1-carbonyl)piperidin-4-yl]methyl]-6-pyrazol-1-ylpyridazin-3-one; 2-[[1-[2-(2-chlorophenyl)acetyl]piperidin-4-yl]methyl]-6-pyrazol-1-ylpyridazin-3-one; 2-[[1-(6-cyclopropylpyrimidin-4-yl)piperidin-4-yl]methyl]-6-(1,2,4-triazol-1-yl)pyridazin-3-one; 2-[(1-methylsulfonylpiperidin-4-yl)methyl]-6-pyrazol-1-ylpyridazin-3-one; 2-[[1-(4-fluorobenzoyl)piperidin-4-yl]methyl]-6-pyrazol-1-ylpyridazin-3-one; 6-(1,2,4-triazol-1-yl)-2-[[1-[6-(trifluoromethyl)pyrimidin-4-yl]piperidin-4-yl]methyl]pyridazin-3-one; 6-(2,4-dimethylthiazol-5-yl)-2-((1-(pyrido[2,3-d]pyrimidin-4-yl)piperidin-4-yl)methyl)pyridazin-3(2H)-one; 2-((1-(7-fluoroquinazolin-4-yl)piperidin-4-yl)methyl)-6-(1H-pyrazol-1-yl)pyridazin-3(2H)-one; 6-(2,4-dimethylthiazol-5-yl)-2-((1-(6-methylpyrimidin-4-yl)piperidin-4-yl)methyl)pyridazin-3(2H)-one; 6-(2,4-dimethylthiazol-5-yl)-2-((1-(2-methylpyrido[3,4-d]pyrimidin-4-yl)piperidin-4-yl)methyl)pyridazin-3(2H)-one; 2-((1-(9H-purin-6-yl)piperidin-4-yl)methyl)-6-(2,4-dimethylthiazol-5-yl)pyridazin-3(2H)-one; 2-((1-(6-fluoroquinazolin-4-yl)piperidin-4-yl)methyl)-6-(1H-1,2,4-triazol-1-yl)pyridazin-3(2H)-one; 2-((1-(1,6-dimethyl-1H-pyrazolo[3,4-d]pyrimidin-4-yl)azetidin-3-yl)methyl)-6-(pyridin-4-yl)pyridazin-3(2H)-one; 2-(1-(1,6-dimethyl-1H-pyrazolo[3,4-d]pyrimidin-4-yl)azetidin-3-yl)-6-(pyridin-4-yl)pyridazin-3(2H)-one; 6-(1H-pyrazol-1-yl)-2-((1-(3-(trifluoromethyl)-[1,2,4]triazolo[4,3-b]pyridazin-6-yl)piperidin-4-yl)methyl)pyridazin-3(2H)-one; 6-(1H-1,2,4-triazol-1-yl)-2-((1-(3-(trifluoromethyl)-[1,2,4]triazolo[4,3-b]pyridazin-6-yl)piperidin-4-yl)methyl)pyridazin-3(2H)-one; 2-((1-(9H-purin-6-yl)azetidin-3-yl)methyl)-6-(pyridin-4-yl)pyridazin-3(2H)-one; 2-((1-(6-methylpyrazin-2-yl)piperidin-4-yl)methyl)-6-(1H-1,2,4-triazol-1-yl)pyridazin-3(2H)-one; 6-(3,5-dimethyl-1H-pyrazol-1-yl)-2-((1-(3-methyl-[1,2,4]triazolo[4,3-b]pyridazin-6-yl)azetidin-3-yl)methyl)pyridazin-3(2H)-one; 6-(3,5-dimethyl-1H-pyrazol-1-yl)-2-(1-(3-methyl-3H-imidazo[4,5-b]pyridin-2-yl)piperidin-4-yl)pyridazin-3(2H)-one; 6-(2,4-dimethyl-1,3-thiazol-5-yl)-2-[[1-[(5-methyl-1,2-oxazol-3-yl)methyl]piperidin-4-yl]methyl]pyridazin-3-one; N-[2-(7-cyclohexyl-6-imino-13-methyl-2-oxo-1,7,9-triazatricyclo[8.4.0.03,8]tetradeca-3(8),4,9,11,13-pentaen-5-yl)-4-phenyl-1,3-thiazol-5-yl]-4-methoxybenzamide; 2-[[1-(1,3-benzoxazol-2-yl)piperidin-4-yl]methyl]-6-(2,4-dimethyl-1,3-thiazol-5-yl)pyridazin-3-one; 2-(3-cyclopropyl-6-oxopyridazin-1-yl)-N-[2-[3-(2,4-dimethyl-1,3-thiazol-5-yl)-6-oxopyridazin-1-yl]ethyl]acetamide; 6-(2,4-dimethylthiazol-5-yl)-2-((1-(6-fluorobenzo[d]oxazol-2-yl)piperidin-4-yl)methyl)pyridazin-3(2H)-one; N-[2-[3-(2,4-dimethyl-1,3-thiazol-5-yl)-6-oxopyridazin-1-yl]ethyl]-2-(4-oxoquinazolin-3-yl)acetamide; N-[2-[3-(2,4-dimethyl-1,3-thiazol-5-yl)-6-oxopyridazin-1-yl]ethyl]-2-(1-methyltetrazol-5-yl)sulfanylacetamide; N-[2-[3-(2,4-dimethyl-1,3-thiazol-5-yl)-6-oxopyridazin-1-yl]ethyl]thiophene-2-sulfonamide; N-[2-[3-(2,4-dimethyl-1,3-thiazol-5-yl)-6-oxopyridazin-1-yl]ethyl]-2,3-dihydro-1,4-benzodioxine-6-sulfonamide; N-[2-[3-(2,4-dimethyl-1,3-thiazol-5-yl)-6-oxopyridazin-1-yl]ethyl]-3,5-dimethyl-1,2-oxazole-4-sulfonamide; 2-(benzimidazol-1-yl)-N-[2-[3-(2,4-dimethyl-1,3-thiazol-5-yl)-6-oxopyridazin-1-yl]ethyl]acetamide; N-[2-[3-(2,4-dimethyl-1,3-thiazol-5-yl)-6-oxopyridazin-1-yl]ethyl]-1-methylsulfonylpiperidine-4-carboxamide; 3-(3,5-dimethyl-1,2-oxazol-4-yl)-N-[2-[3-(2,4-dimethyl-1,3-thiazol-5-yl)-6-oxopyridazin-1-yl]ethyl]propenamide; 2-(1,2-benzoxazol-3-yl)-N-[2-[3-(2,4-dimethyl-1,3-thiazol-5-yl)-6-oxopyridazin-1-yl]ethyl]acetamide; 2-(1,3-benzothiazol-2-ylsulfanyl)-N-[2-[3-(2,4-dimethyl-1,3-thiazol-5-yl)-6-oxopyridazin-1-yl]ethyl]acetamide; N-[2-[3-(2,4-dimethyl-1,3-thiazol-5-yl)-6-oxopyridazin-1-yl]ethyl]-4-methylthiadiazole-5-carboxamide; N-[2-[3-(2,4-dimethyl-1,3-thiazol-5-yl)-6-oxopyridazin-1-yl]ethyl]-1-thiophen-2-ylcyclopentane-1-carboxamide; 3-(benzenesulfonyl)-N-[2-[3-(2,4-dimethyl-1,3-thiazol-5-yl)-6-oxopyridazin-1-yl]ethyl]propenamide; 1-[2-[3-(2,4-dimethyl-1,3-thiazol-5-yl)-6-oxopyridazin-1-yl]ethyl]-3-(naphthalen-1-ylmethyl)urea; 1-[2-[3-(2,4-dimethyl-1,3-thiazol-5-yl)-6-oxopyridazin-1-yl]ethyl]-3-(2-methoxyphenyl)urea; N-[2-[3-(2,4-dimethyl-1,3-thiazol-5-yl)-6-oxopyridazin-1-yl]ethyl]furan-2-carboxamide; and pharmaceutically accepted salts thereof.

Particular compounds of formula (I) of the present invention are those selected from the group consisting of: 2-((1-(5H-pyrazolo[3,4-d]pyrimidin-4-yl)piperidin-4-yl)methyl)-6-(2,4-dimethylthiazol-5-yl)pyridazin-3(2H)-one; 6-(2,4-dimethyl-1,3-thiazol-5-yl)-2-[[1-(7H-purin-6-yl)piperidin-4-yl]methyl]pyridazin-3-one; 6-(2,4-dimethyl-1,3-thiazol-5-yl)-2-[[1-(6-methylpyrimidin-4-yl)piperidin-4-yl]methyl]pyridazin-3-one; 6-(2,4-dimethyl-1,3-thiazol-5-yl)-2-[[1-(2-methylpyrido[3,4-d]pyrimidin-4-yl)piperidin-4-yl]methyl]pyridazin-3-one; 6-(2,4-dimethyl-1,3-thiazol-5-yl)-2-[(1-pyrazolo[1,5-a]pyrimidin-5-ylpiperidin-4-yl)methyl]pyridazin-3-one; 6-(2,4-dimethyl-1,3-thiazol-5-yl)-2-[[1-(2-methylpyrimidin-4-yl)piperidin-4-yl]methyl]pyridazin-3-one; 6-(2,4-dimethyl-1,3-thiazol-5-yl)-2-[[1-(2-propan-2-ylpyrimidin-4-yl)piperidin-4-yl]methyl]pyridazin-3-one; 6-(2,4-dimethyl-1,3-thiazol-5-yl)-2-[[1-(9-methylpurin-6-yl)piperidin-4-yl]methyl]pyridazin-3-one; 6-(2,4-dimethylthiazol-5-yl)-2-((1-(6-methyl-3H-pyrrolo[3,2-d]pyrimidin-4-yl)piperidin-4-yl)methyl)pyridazin-3(2H)-one; 6-(2,4-dimethyl-1,3-thiazol-5-yl)-2-[[1-(6-ethyl-5-fluoropyrimidin-4-yl)piperidin-4-yl]methyl]pyridazin-3-one; 6-(2,4-dimethyl-1,3-thiazol-5-yl)-2-[(1-thieno[2,3-d]pyrimidin-4-ylpiperidin-4-yl)methyl]pyridazin-3-one; 6-(2,4-dimethylthiazol-5-yl)-2-((1-(imidazo[1,2-b]pyridazin-6-yl)piperidin-4-yl)methyl)pyridazin-3(2H)-one; 2-[4-[[3-(2,4-dimethyl-1,3-thiazol-5-yl)-6-oxopyridazin-1-yl]methyl]piperidin-1-yl]pyridine-4-carbonitrile; 6-[4-[[3-(2,4-dimethyl-1,3-thiazol-5-yl)-6-oxopyridazin-1-yl]methyl]piperidin-1-yl]pyridine-3-carbonitrile; 6-(2,4-dimethyl-1,3-thiazol-5-yl)-2-[(1-thieno[3,2-d]pyrimidin-4-ylpiperidin-4-yl)methyl]pyridazin-3-one; 2-[4-[[3-(2,4-dimethyl-1,3-thiazol-5-yl)-6-oxopyridazin-1-yl]methyl]piperidin-1-yl]pyridine-3-carbonitrile; 6-(2,4-dimethylthiazol-5-yl)-2-((1-(6-(trifluoromethyl)pyridin-2-yl)piperidin-4-yl)methyl)pyridazin-3(2H)-one; 6-(2,4-dimethylthiazol-5-yl)-2-((1-(4-(trifluoromethyl)pyridin-2-yl)piperidin-4-yl)methyl)pyridazin-3(2H)-one; 6-(2,4-dimethyl-1,3-thiazol-5-yl)-2-[[1-(5-methylpyrimidin-2-yl)piperidin-4-yl]methyl]pyridazin-3-one; 6-(2,4-dimethyl-1,3-thiazol-5-yl)-2-[(1-pyrimidin-2-ylpiperidin-4-yl)methyl]pyridazin-3-one; 6-(2,4-dimethylthiazol-5-yl)-2-((1-(5-(trifluoromethyl)pyridin-2-yl)piperidin-4-yl)methyl)pyridazin-3(2H)-one; 6-(2,4-dimethyl-1,3-thiazol-5-yl)-2-[[1-(3-methyl-[1,2,4]triazolo[4,3-b]pyridazin-6-yl)piperidin-4-yl]methyl]pyridazin-3-one; 6-(2,4-dimethyl-1,3-thiazol-5-yl)-2-[(1-pyrazolo[1,5-a]pyrazin-4-ylpiperidin-4-yl)methyl]pyridazin-3-one; 6-(2,4-dimethylthiazol-5-yl)-2-((1-(3-(trifluoromethyl)pyridin-2-yl)piperidin-4-yl)methyl)pyridazin-3(2H)-one; 2-[[1-(5-chloropyrimidin-2-yl)piperidin-4-yl]methyl]-6-(2,4-dimethyl-1,3-thiazol-5-yl)pyridazin-3-one; 6-(2,4-dimethyl-1,3-thiazol-5-yl)-2-[[1-(4-methylpyrimidin-2-yl)piperidin-4-yl]methyl]pyridazin-3-one; 6-(2,4-dimethyl-1,3-thiazol-5-yl)-2-[(1-pyrido[2,3-d]pyrimidin-4-ylpiperidin-4-yl)methyl]pyridazin-3-one; 2-[[1-(4,6-dimethylpyrimidin-2-yl)piperidin-4-yl]methyl]-6-(2,4-dimethyl-1,3-thiazol-5-yl)pyridazin-3-one; 6-(2,4-dimethyl-1,3-thiazol-5-yl)-2-[[1-[(5-methyl-1,2-oxazol-3-yl)methyl]piperidin-4-yl]methyl]pyridazin-3-one; 6-(2,4-dimethylthiazol-5-yl)-2-((1-(5,6,7,8-tetrahydroquinazolin-4-yl)piperidin-4-yl)methyl)pyridazin-3(2H)-one; 6-(2,4-dimethyl-1,3-thiazol-5-yl)-2-[(1-quinoxalin-2-ylpiperidin-4-yl)methyl]pyridazin-3-one; 6-(2,4-dimethyl-1,3-thiazol-5-yl)-2-[[1-(5-fluoropyrimidin-2-yl)piperidin-4-yl]methyl]pyridazin-3-one; 5-[4-[[3-(2,4-dimethyl-1,3-thiazol-5-yl)-6-oxopyridazin-1-yl]methyl]piperidin-1-yl]pyridine-2-carbonitrile; 6-(2,4-dimethyl-1,3-thiazol-5-yl)-2-[[1-(2-methylpyrazolo[1,5-a]pyrazin-4-yl)piperidin-4-yl]methyl]pyridazin-3-one; 2-[4-[[3-(2,4-dimethyl-1,3-thiazol-5-yl)-6-oxopyridazin-1-yl]methyl]piperidin-1-yl]pyrimidine-4-carbonitrile; 6-(2,4-dimethyl-1,3-thiazol-5-yl)-2-[[1-(4-methoxypyrimidin-2-yl)piperidin-4-yl]methyl]pyridazin-3-one; 6-(2,4-dimethyl-1,3-thiazol-5-yl)-2-(piperidin-4-ylmethyl)pyridazin-3-one; N-[2-(7-cyclohexyl-6-imino-13-methyl-2-oxo-1,7,9-triazatricyclo[8.4.0.03,8]tetradeca-3(8),4,9,11,13-pentaen-5-yl)-4-phenyl-1,3-thiazol-5-yl]-4-methoxybenzamide; 3-(4-((3-(2,4-dimethylthiazol-5-yl)-6-oxopyridazin-1(6H)-yl)methyl)piperidin-1-yl)pyrazine-2-carbonitrile; 4-[[4-[[3-(2,4-dimethyl-1,3-thiazol-5-yl)-6-oxopyridazin-1-yl]methyl]piperidin-1-yl]methyl]benzonitrile; 6-(2,4-dimethylthiazol-5-yl)-2-((1-(2-fluorobenzyl)piperidin-4-yl)methyl)pyridazin-3(2H)-one; 6-(2,4-dimethylthiazol-5-yl)-2-((1-(4-fluorobenzyl)piperidin-4-yl)methyl)pyridazin-3(2H)-one; 6-(2,4-dimethylthiazol-5-yl)-2-((1-(3-fluorobenzyl)piperidin-4-yl)methyl)pyridazin-3(2H)-one; 2-((1-(2-chlorobenzyl)piperidin-4-yl)methyl)-6-(2,4-dimethylthiazol-5-yl)pyridazin-3(2H)-one; 2-((1-(4-chlorobenzyl)piperidin-4-yl)methyl)-6-(2,4-dimethylthiazol-5-yl)pyridazin-3(2H)-one; 2-((1-(3-chlorobenzyl)piperidin-4-yl)methyl)-6-(2,4-dimethylthiazol-5-yl)pyridazin-3(2H)-one; 6-(2,4-dimethyl-1,3-thiazol-5-yl)-2-[[1-(3-methylquinoxalin-2-yl)piperidin-4-yl]methyl]pyridazin-3-one; 6-(2,4-dimethyl-1,3-thiazol-5-yl)-2-[[1-(2-methyl-6,7-dihydro-5H-cyclopenta[d]pyrindin-4-yl)piperidin-4-yl]methyl]pyridazin-3-one; 2-[[1-[(2,5-difluorophenyl)methyl]piperidin-4-yl]methyl]-6-(2,4-dimethyl-1,3-thiazol-5-yl)pyridazin-3-one; 2-[[1-[(2,4-difluorophenyl)methyl]piperidin-4-yl]methyl]-6-(2,4-dimethyl-1,3-thiazol-5-yl)pyridazin-3-one; 2-[[1-[(3,4-difluorophenyl)methyl]piperidin-4-yl]methyl]-6-(2,4-dimethyl-1,3-thiazol-5-yl)pyridazin-3-one; 2-((1-(3,5-difluorobenzyl)piperidin-4-yl)methyl)-6-(2,4-dimethylthiazol-5-yl)pyridazin-3(2H)-one; 2-[[1-[(2-chloro-6-fluorophenyl)methyl]piperidin-4-yl]methyl]-6-(2,4-dimethyl-1,3-thiazol-5-yl)pyridazin-3-one; 2-[[1-(1,3-benzoxazol-2-yl)piperidin-4-yl]methyl]-6-(2,4-dimethyl-1,3-thiazol-5-yl)pyridazin-3-one; 2-(3-cyclopropyl-6-oxopyridazin-1-yl)-N-[2-[3-(2,4-dimethyl-1,3-thiazol-5-yl)-6-oxopyridazin-1-yl]ethyl]acetamide; tert-butyl 4-[[3-(2,4-dimethyl-1,3-thiazol-5-yl)-6-oxopyridazin-1-yl]methyl]piperidine-1-carboxylate; 6-(2,4-dimethylthiazol-5-yl)-2-((1-(6-fluorobenzo[d]oxazol-2-yl)piperidin-4-yl)methyl)pyridazin-3(2H)-one; N-[2-[3-(2,4-dimethyl-1,3-thiazol-5-yl)-6-oxopyridazin-1-yl]ethyl]-2-(4-oxoquinazolin-3-yl)acetamide; N-[2-[3-(2,4-dimethyl-1,3-thiazol-5-yl)-6-oxopyridazin-1-yl]ethyl]-2-(1-methyltetrazol-5-yl)sulfanylacetamide; N-[2-[3-(2,4-dimethyl-1,3-thiazol-5-yl)-6-oxopyridazin-1-yl]ethyl]thiophene-2-sulfonamide; N-[2-[3-(2,4-dimethyl-1,3-thiazol-5-yl)-6-oxopyridazin-1-yl]ethyl]-2,3-dihydro-1,4-benzodioxine-6-sulfonamide; N-[2-[3-(2,4-dimethyl-1,3-thiazol-5-yl)-6-oxopyridazin-1-yl]ethyl]-3,5-dimethyl-1,2-oxazole-4-sulfonamide; 2-(benzimidazol-1-yl)-N-[2-[3-(2,4-dimethyl-1,3-thiazol-5-yl)-6-oxopyridazin-1-yl]ethyl]acetamide; N-[2-[3-(2,4-dimethyl-1,3-thiazol-5-yl)-6-oxopyridazin-1-yl]ethyl]-1-methylsulfonylpiperidine-4-carboxamide; 3-(3,5-dimethyl-1,2-oxazol-4-yl)-N-[2-[3-(2,4-dimethyl-1,3-thiazol-5-yl)-6-oxopyridazin-1-yl]ethyl]propanamide; 2-(1,2-benzoxazol-3-yl)-N-[2-[3-(2,4-dimethyl-1,3-thiazol-5-yl)-6-oxopyridazin-1-yl]ethyl]acetamide; 2-(1,3-benzothiazol-2-ylsulfanyl)-N-[2-[3-(2,4-dimethyl-1,3-thiazol-5-yl)-6-oxopyridazin-1-yl]ethyl]acetamide; N-[2-[3-(2,4-dimethyl-1,3-thiazol-5-yl)-6-oxopyridazin-1-yl]ethyl]-4-methylthiadiazole-5-carboxamide; N-[2-[3-(2,4-dimethyl-1,3-thiazol-5-yl)-6-oxopyridazin-1-yl]ethyl]-1-thiophen-2-ylcyclopentane-1-carboxamide; 3-(benzenesulfonyl)-N-[2-[3-(2,4-dimethyl-1,3-thiazol-5-yl)-6-oxopyridazin-1-yl]ethyl]propanamide; 1-[2-[3-(2,4-dimethyl-1,3-thiazol-5-yl)-6-oxopyridazin-1-yl]ethyl]-3-(naphthalen-1-ylmethyl)urea; 1-[2-[3-(2,4-dimethyl-1,3-thiazol-5-yl)-6-oxopyridazin-1-yl]ethyl]-3-(2-methoxyphenyl)urea; N-[2-[3-(2,4-dimethyl-1,3-thiazol-5-yl)-6-oxopyridazin-1-yl]ethyl]furan-2-carboxamide; and pharmaceutically accepted salts thereof.

Particular compounds of formula (I) of the present invention are those selected from the group consisting of: 6-(2,4-dimethylthiazol-5-yl)-2-((1-(pyrido[2,3-d]pyrimidin-4-yl)piperidin-4-yl)methyl)pyridazin-3(2H)-one; 2-((1-(7-fluoroquinazolin-4-yl)piperidin-4-yl)methyl)-6-(1H-pyrazol-1-yl)pyridazin-3(2H)-one; 6-(2,4-dimethylthiazol-5-yl)-2-((1-(6-methylpyrimidin-4-yl)piperidin-4-yl)methyl)pyridazin-3(2H)-one; 6-(2,4-dimethylthiazol-5-yl)-2-((1-(2-methylpyrido[3,4-d]pyrimidin-4-yl)piperidin-4-yl)methyl)pyridazin-3(2H)-one; 2-((1-(9H-purin-6-yl)piperidin-4-yl)methyl)-6-(2,4-dimethylthiazol-5-yl)pyridazin-3(2H)-one; 2-((1-(6-fluoroquinazolin-4-yl)piperidin-4-yl)methyl)-6-(1H-1,2,4-triazol-1-yl)pyridazin-3(2H)-one; 2-((1-(1,6-dimethyl-TH-pyrazolo[3,4-d]pyrimidin-4-yl)azetidin-3-yl)methyl)-6-(pyridin-4-yl)pyridazin-3(2H)-one; 2-(1-(1,6-dimethyl-1H-pyrazolo[3,4-d]pyrimidin-4-yl)azetidin-3-yl)-6-(pyridin-4-yl)pyridazin-3(2H)-one; 6-(1H-pyrazol-1-yl)-2-((1-(3-(trifluoromethyl)-[1,2,4]triazolo[4,3-b]pyridazin-6-yl)piperidin-4-yl)methyl)pyridazin-3(2H)-one; 6-(1H-1,2,4-triazol-1-yl)-2-((1-(3-(trifluoromethyl)-[1,2,4]triazolo[4,3-b]pyridazin-6-yl)piperidin-4-yl)methyl)pyridazin-3(2H)-one; 2-((1-(9H-purin-6-yl)azetidin-3-yl)methyl)-6-(pyridin-4-yl)pyridazin-3(2H)-one; 2-((1-(6-methylpyrazin-2-yl)piperidin-4-yl)methyl)-6-(1H-1,2,4-triazol-1-yl)pyridazin-3(2H)-one; 6-(3,5-dimethyl-1H-pyrazol-1-yl)-2-((1-(3-methyl-[1,2,4]triazolo[4,3-b]pyridazin-6-yl)azetidin-3-yl)methyl)pyridazin-3(2H)-one; 6-(3,5-dimethyl-1H-pyrazol-1-yl)-2-(1-(3-methyl-3H-imidazo[4,5-b]pyridin-2-yl)piperidin-4-yl)pyridazin-3(2H)-one; and pharmaceutically accepted salts thereof.

Particular embodiments of the invention relate to compounds of formula (I) wherein j is 2 and k is 2, which is represented by formula (II):

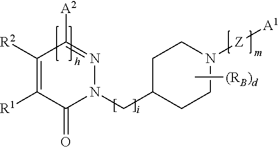

wherein
A¹ is hydrogen, C₁₋₇-alkyl, C₂₋₇-alkenyl, C₃₋₈-cycloalkyl, aryl, or heterocyclyl;
A² is hydrogen, C₁₋₇-alkyl, C₂₋₇-alkenyl, C₃₋₈-cycloalkyl, aryl, or heterocyclyl;
R¹ is hydrogen, halo, C₁₋₇-alkyl, C₂₋₇-alkenyl, C₃₋₈-cycloalkyl, aryl, or heterocyclyl;
R² is hydrogen, halo, C₁₋₇-alkyl, C₂₋₇-alkenyl, C₃₋₈-cycloalkyl, aryl, or heterocyclyl;
or A² and R² together form C₁₋₇-alkylene, or C₂₋₇-alkenylene;
Z is C₁₋₇-alkylene, C₂₋₇-alkenylene, CH₂—, (CH₂)₂—, —CH₂(CH)₂—, —C(═O)—, —C(═O)CH₂—, —C(═O)(CH₂)₂—, —C(═O)CH₂O—, —C(═O)(CH₂)₂O—, —C(═O)CH(CH₃)O—, —C(═O)O—, —C(═O)OCH₂—, —C(═O)O(CH₂)₂—, —C(═O)NH—, —C(═O)NHCH₂—, —C(═O)NH(CH₂)₂—, —S(═O)₂—, —S(═O)₂CH₂—, or —S(═O)₂(CH)₂—;
h is 1, 2, or 3;
i is 0, 1 or 2;
m is 0 or 1;
R_{B} represents non-hydrogen substituent(s), wherein each R_{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein the R_{B} substituent(s), if any, is covalently attached to any ring atom(s) in place of hydrogen, provided the maximum valency of the ring atom(s) to which R_{B} is attached is not exceeded;
d is 0, 1, 2, 3, 4, 5, 6, 7, 8, or 9;
wherein C₁₋₇-alkyl, C₂₋₇-alkenyl, C₁₋₇-alkylene, C₂₋₇-alkenylene, C₃₋₈-cycloalkyl, aryl, and heterocyclyl, are each independently substituted or unsubstituted;
and pharmaceutically acceptable salts thereof.

A particular embodiment of the present invention relates to compounds of formula (II), wherein

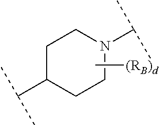

is selected from the group of:

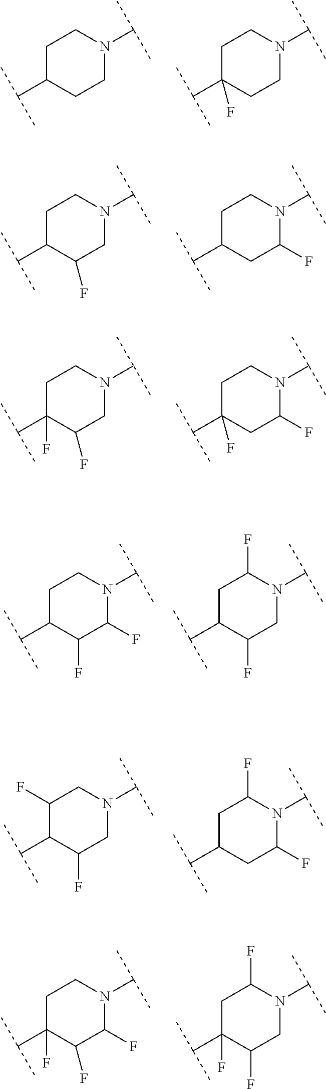

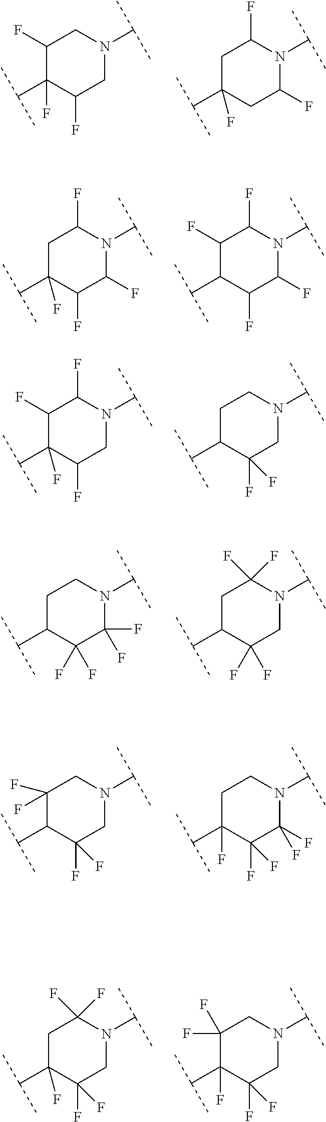

Particular compounds of formula (I) of the present invention are those selected from the group consisting of
6-(2,4-dimethyl-1,3-thiazol-5-yl)-2-(3-fluoro-1-pyrido[2,3-d]pyrimidin-4-ylpiperidin-4-yl)methylpyridazin-3-one;
6-(2,4-dimethyl-1,3-thiazol-5-yl)-2-(3,3-difluoro-1-pyrido[2,3-d]pyrimidin-4-ylpiperidin-4-yl)methylpyridazin-3-one;
6-(2,4-dimethyl-1,3-thiazol-5-yl)-2-(2-fluoro-1-pyrido[2,3-d]pyrimidin-4-ylpiperidin-4-yl)methylpyridazin-3-one;
6-(2,4-dimethyl-1,3-thiazol-5-yl)-2-(2,2-difluoro-1-pyrido[2,3-d]pyrimidin-4-ylpiperidin-4-yl)methylpyridazin-3-one;
6-(2,4-dimethyl-1,3-thiazol-5-yl)-2-(2,3-difluoro-1-pyrido[2,3-d]pyrimidin-4-ylpiperidin-4-yl)methylpyridazin-3-one;
6-(2,4-dimethyl-1,3-thiazol-5-yl)-2-(2,4-difluoro-1-pyrido[2,3-d]pyrimidin-4-ylpiperidin-4-yl)methylpyridazin-3-one;
6-(2,4-dimethyl-1,3-thiazol-5-yl)-2-(2,5-difluoro-1-pyrido[2,3-d]pyrimidin-4-ylpiperidin-4-yl)methylpyridazin-3-one;
6-(2,4-dimethyl-1,3-thiazol-5-yl)-2-(2,6-difluoro-1-pyrido[2,3-d]pyrimidin-4-ylpiperidin-4-yl)methylpyridazin-3-one;
6-(2,4-dimethyl-1,3-thiazol-5-yl)-2-(3,4-difluoro-1-pyrido[2,3-d]pyrimidin-4-ylpiperidin-4-yl)methylpyridazin-3-one;
6-(2,4-dimethyl-1,3-thiazol-5-yl)-2-(3,5-difluoro-1-pyrido[2,3-d]pyrimidin-4-ylpiperidin-4-yl)methylpyridazin-3-one;
   and pharmaceutically accepted salts thereof.

The formulae of the compounds disclosed herein should be interpreted as encompassing all possible stereoisomers, enantiomers, or epimers of the compounds unless the formula indicates a specific stereoisomer, enantiomer, or epimer. The formulae of the compounds disclosed herein should be interpreted as encompassing salts, esters, amides, or solvates thereof of the compounds.

In certain embodiments, provided herein are compounds that are similar to a compound of formula (I) disclosed herein. Similarity between small molecule compounds can be determined using methods well known in the art, such as for example, deriving the Tanimoto index, Dice index, Cosine coefficient or Soergel distance etc. Such methods are described in Bajusz et al. (Bajusz et al., Journal of Cheminformatics, 7: Article number 20 (2015)), which together with the references cited therein, are incorporated herein in their entirety. In some embodiments, provided herein are compounds which have a Tanimoto index of at least 0.70, 0.75, 0.80, 0.85, 0.90, 0.95, 0.96, 0.97, 0.98, 0.99, or 1.00 compared to a compound of formula (I) disclosed herein.

Compounds of formula (I) disclosed herein can be developed and produced by any number of methods known to a person who is skilled in the art (see below for specific methods).

### Targets, Target Regions, Target Segments, and Nucleobase Sequences

In particular embodiments, a compound of formula (I) disclosed herein may target a nucleic acid, wherein the target nucleic acid is an RNA encoding MYC, or part thereof. RNA-targeting small molecules are useful as they combine the advantages of small molecule drugs (see Background) with the advantages of targeting RNA, for instance, allowing for an expanded target scope beyond protein-coding genes to include non-coding regulatory RNAs, as well as allowing for the targeting of “undruggable” proteins that lack deep binding pockets. In certain embodiments, target nucleic acids may comprise nucleobase sequences encoding MYC, including but not limited to RefSeq Accession No. NC_000008.11 truncated from nucleotides 127735434 to 127742951 (incorporated herein as SEQ ID NO: 1), RefSeq Accession No. NM_002467.6 (incorporated herein as SEQ ID NO: 2), and RefSeq Accession No. NM_001354870.1 (incorporated herein as SEQ ID NO: 3), or part thereof, wherein adenine bases (A) are interchangeable with uracil bases (U). In some embodiments, a compound of formula (I) disclosed herein may also target certain other RNA transcript variants encoding MYC, or part thereof.

In certain embodiments, a compound of formula (I) disclosed herein may target at least one target region within a target nucleic acid. A target region is a structurally or functionally defined region of the target nucleic acid. Examples of a target region include but are not limited to an exon, an intron, an exon-intron junction, an intron-exon junction, an exon-exon junction, a 3′ untranslated region (3′ UTR), a 5′ untranslated region (5′ UTR), a translation initiation region, a translation termination region, a 5′ donor splice site, a 3′ acceptor splice site, a start codon, an upstream open reading frame (ORF), a repeat region, a hexanucleotide repeat expansion, a splice enhancer region, an exonic splicing enhancer (ESE), a splice suppressor region, an exonic splicing silencer (ESS), an intronic splicing enhancer (ISE), an intronic splicing silencer (ISS), a RNA destabilization motif, a RNA stabilization motif, a miRNA binding site, a RNA-binding protein (RBP) site, or other defined nucleic acid region. Information on such structurally or functionally defined target regions in RNA, such as MYC RNA, may in part be obtained by accession number from databases such as NCBI, GENBANK, and ENSEMBL, as well as references therein, and such information is incorporated herein in their entirety.

A target region can contain one or more overlapping or non-overlapping target segments. In certain embodiments, target segments within a target region are less than 10000, 5000, 2500, 1000, 500, 250, 100, or 50 nucleotides in length. In some embodiments, a compound of formula (T) disclosed herein may target one target segment within a target region. In some embodiments, a compound of formula (I) disclosed herein may target two or more overlapping or non-overlapping target segments within the same target region. In other embodiments, a compound of formula (I) disclosed herein may target two or more target segments originating from different target regions.

A suitable target segment can specifically include a certain structurally or functionally defined target region, such as, for example, a start codon or a stop codon. The determination of suitable target segments can include a comparison of the nucleobase sequence of the target segment to other sequences throughout the transcriptome. For example, the BLAST algorithm can be used to identify regions of similarity amongst different sequences. This comparison can enable the selection of target segments that are unique in order to develop compounds that specifically target a target nucleic acid with minimal non-specific targeting of other nucleic acids (i.e., off-targets).

In some embodiments, targeting includes determination of at least one target segment within a target nucleic acid to which a compound can interact in order to produce a desired effect. The desired effect can be a decrease in stability of the target nucleic acid, leading to a decrease in RNA level and a corresponding reduction in its encoded protein levels. The desired effect can be an increase in stability of the target nucleic acid, leading to an increase in RNA level and a corresponding increase in its encoded protein levels. The desired effect can also be a change in translation efficiency from the target nucleic acid, leading to no changes in its RNA level, but an increase or decrease in its encoded protein levels. The desired effect can also be a phenotypic change associated with a change in RNA levels of a target nucleic acid or change in protein levels encoded by the target nucleic acid. In certain embodiments, the desired effect of using a compound of formula (I) disclosed herein to target at least one target segment within a target nucleic acid encoding MYC, is a reduction in MYC RNA levels. In other embodiments, the desired effect of using a compound of formula (I) disclosed herein to target at least one target segment within a target nucleic acid encoding MYC, is a reduction in MYC protein levels. In yet other embodiments, the desired effect of using a compound of formula (I) disclosed herein to target at least one target segment within a target nucleic acid encoding MYC is a phenotypic change associated with the reduction of MYC RNA or protein levels.

Thus, some embodiments provide for a compound of formula (I) disclosed herein that can inhibit or reduce the expression or activity of MYC. In one embodiment, a compound of formula (T) disclosed herein may target a nucleic acid encoding MYC and inhibit its transcription. In one embodiment, the compound may target a nucleic acid encoding MYC, and degrade or destabilize it. In one embodiment, the compound may target a nucleic acid encoding MYC and inhibit its translation. In yet another embodiment, the compound may target a nucleic acid encoding MYC and modulate its splicing, thereby decreasing the expression or activity of MYC.

In certain embodiments, the inhibition or reduction of MYC RNA or MYC protein may be achieved by targeting MYC-associated genes or pathways. Thus, in certain embodiments, provided herein is a compound of formula (I) that may reduce or inhibit the expression, activity, or signaling of MYC, by targeting and inhibiting at least one gene or pathway that positively regulates or increases the expression, activity, or signaling of MYC. In other embodiments, provided herein is a compound of formula (I) that may reduce or inhibit the expression, activity, or signaling of MYC, by targeting and increasing the expression or activity of at least one gene or pathway that negatively regulates or inhibits the expression of MYC.

### Conjugates and Complexes

In certain embodiments, a compound of formula (I) disclosed herein is conjugated or bonded to, or complexed with, at least one other molecule, such as a peptide or polypeptide, antibody, lipid, sugar, nucleotide or oligonucleotide, other polymer, cleavage agent, transport agent, intercalating agent, molecular beacon, hybridization-triggered crosslinking agent, lipophilic agent, or hydrophilic agent. These conjugates or complexes can provide a number of benefits to the compound, including but not limited to, increased effectiveness or activity, improved delivery to specific tissues or cells, increased oral bioavailability or absorption, enhanced cellular uptake, lowered toxicity, resistance to nuclease degradation, increased half-life or residence time, enhanced pharmacodynamic or pharmacokinetic properties, and/or improved selectivity for a particular target. Certain embodiments can include a combination of one or more of the conjugates or complexes described herein.

In some embodiments, a compound of formula (I) disclosed herein is conjugated to, or complexed with, a protein or other polyamide, amine, or similar molecule. In other embodiments, a compound of formula (I) disclosed herein is conjugated to, or complexed with, a lipid, phospholipid, cholesterol or thiocholesterol, cholic acid, aliphatic chain, hexylamino-carbonyl-oxycholesterol, or another similar molecule. In yet other embodiments, a compound of formula (I) disclosed herein is conjugated to, or complexed with, another organic molecule, such as an ether or thioether, steroid, biotin, phenazine, folate, phenanthridine, anthraquinone, acridine, adamantine acetic acid, palmityl, fluorescein, rhodamine, coumarin, dye or other marker molecule, or other polymer, such as polyethylene glycol. In other embodiments, a compound of formula (I) disclosed herein is conjugated to, or complexed with, another compound with special electromagnetic or optical properties, such as a photo-labile protecting group.

In some embodiments, a compound of formula (I) disclosed herein is conjugated to, or complexed with, another drug or pharmaceutical agent described herein that is used to treat, prevent or ameliorate cancer. In other embodiments, a compound of formula (I) disclosed herein is conjugated to, or complexed with, another drug, such as for example, another drug alleviating pain or other symptoms or improving uptake or delivery, such as blood thinners (e.g., aspirin, warfarin), anti-inflammatory and pain relief drugs (e.g., COX inhibitors, phenylbutazone, ibuprofen, suprofen, fenbufen, ketoprofen, pranoprofen, carprofen, indomethacin, folinic acid, taiprofenic acid, diclofenac, niflumic acid, diazepines or benzodiazepines, barbiturate); or antibacterial, antiviral, antibiotic, or other drug that promotes at least one benefit in a therapeutic setting, including treatment efficacy, symptom alleviation, drug tolerance, or side effect mediation, etc.

In some embodiments, a compound of formula (I) disclosed herein is conjugated to, or complexed with, an antibody or monobody, which recognizes specific antigens that are characteristic of specific cell type(s) (e.g., cancer cells) or tissues to enable delivery of a compound of formula (I) to the specific cell type(s) or tissues. In some embodiments, a compound of formula (I) disclosed herein is conjugated to, or complexed with, a nanobody or nanocarrier, which enables targeted delivery of a compound of formula (I) to specific cell type(s) or tissues. In other embodiments, a compound of formula (I) disclosed herein is conjugated to, or complexed with, another agent promoting transport across cell membranes, or another agent promoting transport across the blood-brain barrier. In some embodiments, a compound of formula (I) disclosed herein is conjugated to, or complexed with, one or more GalNAc residues, which are recognized by the asialoglycoprotein receptor resulting in efficient uptake into cells.

In some embodiments, a conjugate described herein is a pro-drug, wherein it is converted (e.g., by enzymes in the body) after administration into a pharmacologically active drug(s), such as a compound of formula (I) disclosed herein. Pro-drugs are useful, for example, to improve stability, improve solubility, increase permeability, increase drug absorption, extend duration of action, or achieve selective transport of pharmacologically active drug(s), etc. At present, about 10% of drugs approved worldwide are administered as pro-drugs. Pro-drugs are described by Hajnal et al. (Hajnal et al., Acta Medica Marisiensis, 62(3): 356-362 (2016)), Rautio et al. (Rautio et al., Nature Reviews Drug Discovery, 17: 559-587 (2018)), and Rautio et al. (Rautio et al., Wiley-VCH Verlag GmbH & Co. KGaA, Prodrugs and Targeted Delivery: Towards Better ADME Properties, Volume 47 (2011)), which together with references cited therein, are incorporated herein in their entirety.

In some embodiments, a compound of formula (I) disclosed herein may be conjugated to a ribonuclease, such as RNase L, to induce degradation of a target RNA. Such bivalent compounds are known as RIBOTACs (ribonuclease-targeting chimeras) and are described by Dey et al. (Dey et al., Cell Chemical Biology, 26(8): 1047-1049 (2019)), which together with references cited therein, are incorporated herein in their entirety. In other embodiments, a compound of formula (I) disclosed herein may be conjugated to a ligand for E3 ubiquitin ligase to induce ubiquitination of a target protein and its subsequent degradation by the proteasome. Such bivalent compounds are known as PROTACS (proteolysis-targeting chimeras) and are described by Toure et al. (Toure et al., Angew. Chem. Int. Ed., 55: 1966-1973 (2016)), which together with references cited therein, are incorporated herein in their entirety.

### Pharmaceutical Compositions and Methods of Delivery

Some embodiments of the invention provide for pharmaceutical compositions or medicaments comprising a compound of formula (I) disclosed herein, together with a pharmaceutically acceptable carrier, diluent, and/or other substance. Another embodiment relates to methods of using a compound of formula (I) disclosed herein, to prepare such pharmaceutical compositions or medicaments. The carrier, diluent, and/or other substance of the pharmaceutical composition must be “acceptable” in the sense of being compatible with the other ingredients of the composition and not deleterious to the recipients thereof. In one embodiment, the carrier, diluent, and/or other substance is therapeutically inert. The terms “pharmaceutical compositions” and “medicaments” are used herein interchangeably.

Pharmaceutical compositions or medicaments are formulated, dosed, and administered according to good medical practice. A list of common practices is described by Wu & Chen (US 2018/0112272 A¹), which along with references cited therein, are incorporated herein in its entirety. In some embodiments, a pharmaceutical composition or medicament can comprise a formulation prepared by mixing at least one compound of formula (I) disclosed herein with a carrier or excipient. Suitable carriers and excipients are described in more detail in Ansel et al. (Ansel et al., Ansel's Pharmaceutical Dosage Forms and Drug Delivery Systems (2004) Lippincott, Williams & Wilkins, Philadelphia), Gennaro et al. (Gennaro et al., Remington: The Science and Practice of Pharmacy (2000) Lippincott, Williams & Wilkins, Philadelphia), and Rowe et al. (Rowe et al., Handbook of Pharmaceutical Excipients (2005) Pharmaceutical Press, Chicago), which along with references cited therein, are incorporated herein in their entirety.

In some embodiments, the pharmaceutical composition or medicament can comprise at least one compound of formula (I) disclosed herein together with one or more substances common in pharmaceutical preparations such as, for example, adjuvants, anesthetics, antioxidants, buffers, carriers, colorants, diluents, emulsifiers, flavoring agents, glidants, lubricating agents, masking agents, opaquing agents, perfuming agents, pH modifiers, preservatives, processing aids, salts for varying the osmotic pressure, solubilizers, stabilizing agents, surfactants, suspending agents, sweeteners, and wetting agents, etc. The pharmaceutical composition or medicament may also include one or more substances to improve the presentation, and/or to aid in the manufacturing of the pharmaceutical product. Pharmaceutical compositions or medicaments may also comprise additional therapeutically valuable substances. For example, pharmaceutical compositions or medicaments may comprise a mix of two or more compounds that are each therapeutically beneficial, at least one of which is a compound of formula (I) disclosed herein.

In some embodiments, the aforementioned pharmaceutical compositions or medicaments are prepared in unit dosage form for ease of administration and uniformity of dosage. Unit dosage form as used herein refers to physically discrete units suitable as unitary dosages, each unit containing a predetermined quantity of active ingredient (i.e., a compound of formula (I) disclosed in the present invention) calculated to produce a desired therapeutic effect in association with the carrier and/or other substances in the pharmaceutical composition. Examples of such unit dosage forms are tablets (including scored or coated tablets), capsules, pills, powder packets, wafers, suppositories, injectable solutions or suspensions and the like, and segregated multiples thereof.

The dosages at which a compound of formula (I) disclosed herein is administered can vary within wide limits and need to be tailored to the individual requirements of each case. For example, in some embodiments, the daily dosage can range from about 0.01 mg to about 1000 mg per person in the case of orally administered compounds, although the upper and lower limits can be exceeded if necessary.

In certain embodiments, the pharmaceutical compositions or medicaments may be administered by any suitable means, including enteric, epidural, inhalation, intraarterial, intracerebral, intracerebroventricular, intradermal, intralesional (if local treatment is desired), intramuscular, intranasal, intraocular, intraperitoneal, intrapulmonary, intrathecal, intravenous, intravitreal, oral, parenteral, percutaneous, rectal, subcutaneous, topical (including buccal and sublingual), transdermal, transmucosal, and vaginal administration, etc.

The pharmaceutical compositions or medicaments, in some embodiments, may be in any convenient form for administration such as, for example, capsules, conjugates, creams, crystals, dispersions, elixirs, emulsions, nanoparticles, ointments, gels, patches, pills, powders, solutions, sprays, suppositories, suspensions, syrups, or tablets, etc.

The following are examples of pharmaceutical compositions suitable for different routes of administration, and their methods of preparation. In certain embodiments, pharmaceutical compositions for oral administration in liquid forms such as suspensions, syrups, elixirs, emulsions and solutions, may comprise liquid carriers or diluents such as, for example, water, glycols, oils, alcohols and the like. In other embodiments, pharmaceutical compositions for oral administration in solid forms such as powders, pills, capsules and tablets, may comprise solid carriers such as sugars (e.g., mannitol, lactose, sucrose, glucose, sodium saccharine, etc.), magnesium, carbonate, kaolin, diluents, lubricants (e.g., silicon dioxide, stearic acids, magnesium stearate, talcum, etc.), binders, disintegrating agents to facilitate the dissolution of solids (e.g., starches, clays, celluloses, aligns, gums, and polymers, etc.), and the like. Because of their ease of administration, tablets and capsules represent the most advantageous oral dosage unit forms, in which case solid pharmaceutical carriers are obviously employed.

In some embodiments, pharmaceutical compositions that are administered parenterally, for example, in the form of injectable solutions or suspensions, may comprise carriers such as sterile water, though other ingredients may be included, for example, to aid solubility. Injectable solutions may be prepared in which the carrier comprises saline solution, glucose solution or a mixture of saline and glucose solution. Injectable solutions may be formulated in oil for prolonged action. Appropriate oils for this purpose are, for example, peanut oil, sesame oil, cottonseed oil, corn oil, soybean oil, synthetic glycerol esters of long chain fatty acids and mixtures of these and other oils. Injectable suspensions may be prepared using appropriate liquid carriers, suspending agents, and the like. In certain embodiments, pharmaceutical compositions for parenteral administration can be sterile solutions, emulsions or suspensions that are prepared from a solid or lyophilized form prior to administration. In other embodiments, the pharmaceutical composition may contain certain adjuvants, anesthetics, buffering agents, or wetting agents that promote more effective distribution of the composition, facilitate ease of administration of the composition, or improve patient response or wellbeing.

Certain embodiments provide for pharmaceutical compositions for percutaneous administration (e.g., transdermal, or topical), wherein the carrier optionally comprises a penetration enhancing agent and/or a suitable wetting agent, optionally combined with suitable additives of any nature in minor proportions, which additives do not introduce a significant deleterious effect on the skin. Said additives may facilitate the administration via the skin and/or may be helpful for preparing the desired composition. The pharmaceutical composition may be administered in various ways, e.g., as a transdermal patch, as a spot-on, cream, gel or ointment, etc.

Some embodiments provide for pharmaceutical compositions that can be administered transmucosally, such as in the form of a spray or a suppository. Other embodiments provide for pharmaceutical compositions that can be administered by nasal administration (e.g., an inhalant), and/or that can be administered via an aerosol delivery device, such as an atomizer, nebulizer, or vaporizer. The aerosol delivery devices mentioned herein, and other aerosol delivery devices are well known to a person of ordinary skill in the art and are included in various embodiments herein.

Certain embodiments of the invention provide for methods of delivery of a compound of formula (I) disclosed herein, or pharmaceutical compositions thereof, to a cell, a tissue, an animal, or a human subject. In some embodiments, the compounds of formula (I) disclosed herein, or pharmaceutical compositions thereof, are delivered using one of the routes of administration described above, or other routes of administration known to a person of ordinary skill in the art. In some embodiments, a compound of formula (I) disclosed herein, or pharmaceutical compositions thereof, are delivered via a targeted method that introduces or directs the compound or pharmaceutical composition directly to specific cells or tissues involved in a disease, such as for example, cancerous cells or a tumor. Manish and Vimukta (Manish and Vimukta, Research Journal of Chemical Sciences, 1(2): 135-138 (2011)) describe some methods for targeted drug delivery, which along with references cited therein, are incorporated herein by reference in its entirety.

In some embodiments, the methods of delivery of a compound of formula (I) disclosed herein, or pharmaceutical compositions thereof, include naked delivery into a cell, an animal, or a human subject. In some embodiments, the method of delivery is electroporation or permeabilization. In other embodiments, the method of delivery includes the use of a liposomal-based or amine-based transfection reagent. In other embodiments, the method of delivery includes other forms of lipid-mediated transport. In another embodiment, the method of delivery involves the use of membrane fusion. In another embodiment, the method of delivery includes the use of colloids containing polymeric particles or solutions of nanoparticles. Nanoparticles can include certain properties that assist in targeting certain areas for delivery or otherwise promote delivery, such as electromagnetic properties. In another embodiment, the method of delivery includes the use of chemical-mediated transport, including the use of calcium phosphate. In another embodiment, the method of delivery includes peptide-mediated transport, including the use of polylysine. In another embodiment, the method of delivery includes the use of endocytosis. In other embodiments, the method of delivery can include microinjections directly into cells. These methods of delivery can be used with or without aforementioned complexes or conjugates of the compound of formula (I) disclosed herein. Certain complexes or conjugates can improve the rate of delivery or stability of the compound and are included in these embodiments. Other methods of delivery involve standard practices that are known to a person who is skilled in the art and are incorporated in certain embodiments herein.

### Analysis of Activity of Compounds and Pharmaceutical Compositions

A compound of formula (I) disclosed herein, or pharmaceutical compositions thereof, can have variable activity, for example, as defined by percent reduction of a target RNA level, percent reduction of a target protein level, or percent reduction of the activity of a target protein. In some embodiments, the target is MYC. In certain embodiments, a decrease in MYC RNA levels, including but not limited to, RNA from the transcription of MYC genes, and RNA that is translated into MYC protein, are indicative of reduction or inhibition of MYC expression. In particular embodiments, a decrease in levels of one or more MYC transcripts disclosed by SEQ ID NOs: 1-3, is indicative of reduction or inhibition of MYC expression. In some embodiments, a decrease in levels of MYC protein is indicative of reduction or inhibition of MYC expression. In particular embodiments, a decrease in levels of one or more MYC proteins that are translation products of one or more MYC transcripts disclosed by SEQ ID NOs: 1-3 herein, is indicative of reduction or inhibition of MYC expression.

In other embodiments, a decrease in activity of MYC proteins that are translation products of one or more MYC transcripts disclosed by SEQ ID NOs: 1-3 herein is indicative of reduction or inhibition of activity of MYC. Activity of MYC refers to one or more activities that are normally carried out by MYC transcripts or proteins described herein such as, for example, regulation of cell proliferation, cell cycle, cell growth, differentiation, angiogenesis, apoptosis, immunity, stress response, or oncogenesis etc. In certain embodiments, a compound of formula (I) disclosed herein, or pharmaceutical compositions thereof, may selectively target and decrease the levels or activity of one or more particular MYC transcript variants and the proteins encoded by them, such decrease in levels or activity of one or more MYC transcript variants or proteins being indicative of a reduction of MYC expression or activity.

In yet other embodiments, certain phenotypic changes produced as a result of administration of a compound of formula (I) disclosed herein, or pharmaceutical compositions thereof, to cells, animals, or human subjects, can be indicative of a reduction or inhibition of MYC expression or activity, such as for example, cell cycle arrest, decreased cancer cell growth or proliferation, decreased cancer cell viability, decreased cancer cell migration, decreased cancer cell invasion, decreased cancer cell metastasis, decreased angiogenesis, decreased or increased apoptosis, etc.

In certain embodiments, changes to other genes, mRNA, proteins, or pathways in the cell that result from reducing or inhibiting the expression or activity of MYC, and which are well known to a person skilled in the art, are incorporated herein as indicative of reduction or inhibition of MYC expression or activity.

### Analysis of RNA Levels

In certain embodiments, the reduction or inhibition of MYC expression by a compound of formula (I) disclosed herein, or pharmaceutical compositions thereof, can be assessed by measuring the decrease in levels of MYC RNA transcripts. RNA analysis can be carried out on poly(A)+ mRNA or total cellular RNA. Methods of RNA isolation are well known in the art and include, for example, using the TRIZOL Reagent (Invitrogen, Carlsbad, CA) according to the manufacturer's recommended protocols, or using an RNA extraction kit (Qiagen) etc. The target RNA levels can be quantified using methods well known in the art and include, for example, Northern blot analysis, competitive polymerase chain reaction (PCR), or reverse transcription followed by quantitative real-time PCR using the ABI PRISM 7600, 7700, or 7900 Sequence Detection System (PE-Applied Biosystems, Foster City, CA), and the like, according to the manufacturer's instructions.

Prior to quantitative real-time PCR, the isolated RNA first undergoes a reverse transcription reaction to produce complementary DNA (cDNA), which is then used as the substrate for the real-time PCR amplification reaction. Reagents for reverse transcription and real-time PCR can be obtained commercially (e.g., Invitrogen, Carlsbad, CA). The reverse transcription reaction and real-time PCR reactions can be performed sequentially in the same sample well or in different sample wells. The levels of a target gene or RNA that are obtained by real-time PCR can be normalized using either total RNA levels quantified by, for example, RIBOGREEN (Invitrogen, Carlsbad, CA), or normalized using the expression level of a gene whose expression in the cell is more or less stable, such as cyclophilin A or beta actin. Methods of RNA quantification using RIBOGREEN are described in Jones et al. (Jones et al., Analytical Biochemistry, 265: 368-374 (1998)), which together with the references cited therein, are incorporated herein in its entirety. A CYTOFLUOR 4000 instrument (PE Applied Biosystems) can be used to measure RIBOGREEN fluorescence. The expression levels of cyclophilin A or beta actin can be quantified by real-time PCR within the same well as that used for quantifying the levels of target RNA (i.e., by performing a multiplex reaction) or by running it in separate wells. Probes and primers that hybridize to a target nucleic acid encoding MYC can be designed using methods that are well known in the art, and can include the use of software, such as, for example, PRIMER EXPRESS Software (Applied Biosystems, Foster City, CA). Additional methods to measure the decrease in levels of MYC RNA are described in Example 2, which are included in the embodiments herein.

### Analysis of Protein Levels

In certain embodiments, the reduction or inhibition of MYC expression by a compound of formula (I) disclosed herein, or pharmaceutical compositions thereof, can be assessed by measuring the decrease in levels of MYC protein. Several methods for quantifying or measuring protein levels of MYC are well known in the art, such as Western blot analysis, enzyme-linked immunosorbent assay (ELISA), immunoprecipitation, immunocytochemistry, fluorescence activated cell sorting (FACS), immunohistochemistry, protein activity assays, quantitative protein assays, bicinchoninic acid assay (BCA assay) also known as the Smith assay, and the like. Antibodies that are specific for a target protein, such as MYC, can be generated using conventional monoclonal or polyclonal antibody generation methods well known in the art, or identified and obtained commercially from a variety of sources, such as the MSRS catalog of antibodies (Aerie Corporation, Birmingham, MI). Antibodies for the detection of mouse, rat, monkey, and human MYC are available commercially. Additional methods to measure the decrease in levels of MYC protein are described in Example 1, which are included in some embodiments herein.

### In Vitro Testing of Compounds

A compound of formula (I) disclosed herein, or pharmaceutical compositions thereof, can be administered to cultured cells in vitro to evaluate their effects on the expression or activity of target gene(s) (e.g., MYC), or one or more phenotypes such as, for example, cell growth or proliferation, cell viability, cell morphology, cell cycle arrest, cell migration, cell invasion, and apoptosis, etc. In some embodiments, administration of a compound of formula (I) disclosed herein, or pharmaceutical compositions thereof, to cultured cells, leads to the reduction or inhibition of expression of MYC protein (e.g., see Example 1). In other embodiments, administration of a compound of formula (I) disclosed herein, or pharmaceutical compositions thereof, to cultured cells, leads to the reduction or inhibition of expression of MYC RNA (e.g., see Example 2). In some embodiments, administration of a compound of formula (I) disclosed herein, or pharmaceutical compositions thereof, to cultured cells, leads to one or more phenotypes such as for example, decreased cancer cell growth or proliferation, decreased cancer cell viability, and decreased apoptosis, etc. (e.g., see Examples 3-5).

In certain embodiments, the cultured cells for in vitro testing of compounds are animal or human cancer-derived cells such as, for example, breast cancer cells (e.g., MCF7, MDA-MB-231, MDA-MB-468, HS 578T, BT-549 and T-47D, etc.), Burkitt's lymphoma cells (e.g., BL-2, CA-46, etc.), colon cancer cells (e.g., COLO 205, HCC-2998, HCT-116, HCT-15, HT29, KM12 and SW-620, etc.), CNS cells (e.g., SF-268, SF-295, SF-539, SNB-19, SNB-75 and U251, etc.), glioblastoma cells (e.g., U-87, etc.), leukemia cells (e.g., CCRF-CEM, HL-60, K-562, MOLT-4, RPMI-8226 and SR, etc.), liver cancer cells (e.g., HepG2, etc.), melanoma cancer cells (e.g., LOX IMVI, MALME-3M, M14, MDA-MB-435, SK-MEL-2, SK-MEL-28, SK-MEL-5, UACC-257 and UACC-62, etc.), multiple myeloma cells (e.g., MM.1S cells), non-small cell lung cancer cells (e.g., A549, EKVX, HOP-62, HOP-92, NCI-H226, NCI-H23, NCI-H322M, NCI-H460 and NCI-H522, etc.), ovarian cancer cells (e.g., IGR-OV1, OVCAR-3, OVCAR-4, OVCAR-5, OVCAR-8, NCI/ADR-RES and SK-OV-3, etc.), prostate cancer cells (e.g., DU-145 and PC-3, etc.), renal cancer cells (e.g., 786-0, A⁴⁹⁸, ACHN, CAKI-1, RXF 393, SN12C, TK-10 and UO-31, etc.), and small-cell lung cancer cells (e.g., H2171, H82 etc.), etc. In particular embodiments, the cultured cells are derived from a MVC-driven cancer. Examples of cancer-derived cells, including MYC-driven cancer cells, are described in the catalogs of commercial vendors, such as, for example, Clonetics Corporation, Walkersville, MD; American Type Culture Collection, Manassas, VA; Zen-Bio, Inc., Research Triangle Park, NC etc., and are incorporated by reference herein. Such cells are cultured according to the vendor's instructions using commercially available reagents (e.g., Invitrogen Life Technologies, Carlsbad, CA). Cells can be cultured and tested in multi-well plates, for example, 6-well, 24-well, 48-well, 96-well, 384-well plates etc.

The one or more phenotypes resulting from administration of a compound of formula (I) disclosed herein, or pharmaceutical compositions thereof, to cultured cells, such as decrease in cell growth and proliferation, decrease in cell viability, or decrease or increase in apoptosis, can be assessed using any method known to a person skilled in the art. Examples of assays to assess cell growth and proliferation, or cell viability, include colorimetric assays that utilize dyes such as MTT, XTT, MTS, and CCK-8, fluorescence-based assays such as ApoTox-Glo, and luminescence-based assays such as CellTiter-Glo, and the like. Examples of assays to assess apoptosis include ApoTox-Glo, Caspase-Glo (3/7, 8 and 9), Annexin V. and the like. In certain embodiments, the disclosed compounds have an of less than about 10 μM, 5 μM, 1 μM, 0.5 μM, 0.25 μM, 0.1 μM, 0.05 μM, 0.01 μM or lower in the selected assay. In other embodiments, the disclosed compounds decrease the growth or viability of cancer cells, such as MYC-driven cancer cells, by at least 10%, 20%, 30%, 40%, 50%, 60%, 70%, 80%, or 90%, at a concentration of less than about 500 μM, 100 μM, 50 μM, 20 μM, 10 μM, 1 μM, 0.1 μM, 0.05 μM, 0.01 μM, or less. In some embodiments, the disclosed compounds decrease the growth or viability of healthy (non-cancer) cells by not more than 50%, 40%, 30%, 20%, 10%, 5%, or less at a concentration of less than about 100 μM, 50 μM, 20 μM, 10 μM, 1 μM, 0.1 μM, 0.05 μM, 0.01 μM, or less.

### In Vivo Testing of Compounds

A compound of formula (I) disclosed herein, or pharmaceutical compositions thereof, can be administered to animals or human subjects in vivo to evaluate their properties such as, for example, pharmacokinetics profile, safety, maximum tolerated dose (MTD), ADME (absorption, distribution, metabolism, and excretion), pharmacodynamics, effects on the expression or activity of target gene(s) (e.g., MYC), or effects on one or more phenotypes such as, for example, survival, cancer cell growth, cancer metastasis, behavior, body weight, metabolism, etc. In some embodiments, administration of a compound of formula (1) disclosed herein, or pharmaceutical compositions thereof, to animals or human subjects, leads to the reduction or inhibition of expression and/or activity of MYC protein and/or RNA. In some embodiments, administration of a compound of formula (I) disclosed herein, or pharmaceutical compositions thereof, to animals or human subjects, leads to one or more phenotypes such as for example, decrease in tumor volume (i.e., tumor regression), decrease in cancer cell growth or proliferation, decrease in cancer metastasis, increase in animal or human subject survival, or other desired outcome with respect to particular phenotypes (e.g., body weight, metabolism, etc.). In some embodiments, testing can be performed in healthy animals or human subjects. In other embodiments, testing can be performed in disease animal models (e.g., cell line-derived xenograft (CDX) models, patient-derived xenograft (PDX) models, syngeneic models, orthotopic models, humanized models, spontaneous or induced cancer models, etc.), or human subjects diagnosed with cancer.

In certain embodiments, a compound of formula (I) disclosed herein is formulated in a pharmaceutically acceptable diluent, such as phosphate-buffered saline, for administration to animals. Administration includes any route of administration described herein such as, for example, oral, intrathecal, intraperitoneal, intravenous, and subcutaneous, etc. Methods to calculate appropriate dosages of compounds disclosed herein and dosing frequency are well known in the art and depend upon factors such as animal body weight and route of administration. Following the administration of a compound disclosed herein, or pharmaceutical compositions thereof, to the animal, the animals are monitored at defined timepoints for the expression levels of target gene(s) such as MYC, and effects on other phenotypes such as, for example, survival, cancer cell growth, cancer metastasis, behavior, body weight, metabolism, etc. The levels of MYC RNA or MYC protein can be measured in different tissues from the animal, such as, for example, the CSF, plasma, brain, spinal cord, lung, liver, kidney etc., using methods known in the art and described herein. Other modifications are known to those skilled in the art and are considered to be included in the embodiments herein.

In certain embodiments, administration of a compound of formula (I) disclosed herein, or pharmaceutical compositions thereof, results in a decrease of MYC RNA by at least 5, 10, 20, 30, 40, 50, 60, 70, 80, or 90%, or a range defined by any two of these values. In certain embodiments, administration of a compound of formula (I) disclosed herein, or pharmaceutical compositions thereof, results in a decrease of MYC protein by at least 5, 10, 20, 30, 40, 50, 60, 70, 80, or 90%, or a range defined by any two of these values. In certain embodiments, administration of a compound of formula (I) disclosed herein, or pharmaceutical compositions thereof, results in a decrease in tumor size by at least 5, 10, 20, 30, 40, 50, 60, 70, 80, or 90%, or a range defined by any two of these values. In certain embodiments, administration of a compound of formula (I) disclosed herein, or pharmaceutical compositions thereof, results in a decrease in tumor metastasis. In certain embodiments, administration of a compound of formula (I) disclosed herein, or pharmaceutical compositions thereof, results in an increase in animal or human subject survival by at least 5, 10, 20, 30, 40, 50, 60, 70, 80, 85, 90, 95, 96, 97, 98, or 99%, or a range defined by any two of these values.

### Methods of Use and Treatment Methods

Some embodiments provide for methods of reducing the expression of MYC in cells or tissues, comprising administering a compound of formula (I) disclosed herein, or pharmaceutical compositions thereof, to a cell, animal, or human, such that expression of MYC is reduced. Other embodiments provide for methods of reducing the activity of MYC in cells or tissues, comprising administering a compound of formula (I) disclosed herein, or pharmaceutical compositions thereof, to a cell, animal, or human, such that activity of MYC is reduced. Other embodiments provide for methods of achieving one or more phenotypic outcomes such as, for example, decrease in cancer cell growth or proliferation, decrease in cancer cell viability, decrease in apoptosis, decrease in tumor volume (i.e., tumor regression), decrease in cancer metastasis, increase in animal or human subject survival, or other desired outcome with respect to particular phenotypes (e.g., body weight, metabolism, etc.), comprising administering a compound of formula (I) disclosed herein, or pharmaceutical compositions thereof, to a cell, animal, or human. Yet other embodiments provide for methods for treating diseases, such as cancers, comprising administering an effective amount of a compound of formula (I) disclosed herein, or pharmaceutical compositions thereof, to an animal or human. In particular embodiments, the cancer is a MYC-driven cancer.

In certain embodiments, provided herein are methods of treatment of a human subject diagnosed with cancer, such as a MYC-driven cancer, comprising administering a compound of formula (I) disclosed herein, or pharmaceutical compositions thereof, to the human individual. In certain embodiments, provided herein are methods for prophylactically reducing MYC expression or activity in a human subject to prevent diseases such as cancer, wherein the human subject is at risk for developing a cancer, such as a MYC-driven cancer. In other embodiments, a compound of formula (I) disclosed herein, or pharmaceutical compositions thereof, is administered to a human subject to treat or ameliorate the symptoms of cancer.

In particular embodiments, the MYC-driven cancer refers to a C-MYC-driven cancer such as, for example, breast cancer, Burkitt's lymphoma, cervical cancer, colorectal cancer (e.g., colon adenocarcinoma, rectum adenocarcinoma), esophageal carcinoma, gastric cancer (e.g., stomach adenocarcinoma), glioblastoma (e.g., glioblastoma multiforme), head and neck squamous cell carcinoma, leukemia (e.g., myeloid leukemia), liver cancer, lung cancer (e.g., non-small cell lung cancer, small cell lung carcinoma, lung squamous cell carcinoma), non-Burkitt's lymphoma, medulloblastoma, melanoma (e.g., skin cutaneous melanoma, uveal melanoma), mesothelioma, multiple myeloma, neuroblastoma, osteosarcoma, ovarian cancer, pancreatic cancer, prostate cancer, renal cancer (e.g., clear cell renal cell carcinoma), and rhabdomyosarcoma, etc. In particular embodiments, the MYC-driven cancer refers to a N-MYC-driven cancer such as, for example, astrocytoma, brain lower grade glioma, breast cancer, glioblastoma, lung cancer (e.g., small cell lung cancer), medullary thyroid carcinoma, medulloblastoma, neuroblastoma, ovarian cancer, pancreatic cancer, pheochromocytoma and paraganglioma, prostate cancer, retinoblastoma, rhabdomyosarcoma (e.g., alveolar rhabdomyosarcoma), and testicular cancer, etc.

Other embodiments provide for use of a compound of formula (I) disclosed herein, or pharmaceutical compositions thereof, for reducing the expression or activity of MYC. Yet other embodiments provide for use of a compound of formula (I) disclosed herein, or pharmaceutical compositions thereof, for achieving one or more phenotypic outcomes such as, for example, decrease in cancer cell growth or proliferation, decrease in cancer cell viability, decrease in apoptosis, decrease in tumor volume (i.e., tumor regression), decrease in cancer metastasis, increase in animal or human subject survival, or other desired outcome with respect to particular phenotypes (e.g., body weight, metabolism, etc.). Yet other embodiments provide for use of a compound of formula (I) disclosed herein, or pharmaceutical compositions thereof, to treat, prevent or ameliorate cancer, such as a MYC-driven cancer.

Certain embodiments provide for a compound of formula (I) disclosed herein, or pharmaceutical compositions thereof, for use in reducing the expression or activity of MYC. Certain other embodiments provide for a compound of formula (I) disclosed herein, or pharmaceutical compositions thereof, for use in achieving one or more phenotypic outcomes such as, for example, decrease in cancer cell growth or proliferation, decrease in cancer cell viability, decrease in apoptosis, decrease in tumor volume (i.e., tumor regression), decrease in cancer metastasis, increase in animal or human subject survival, or other desired outcome with respect to particular phenotypes (e.g., body weight, metabolism, etc.). Certain other embodiments provide for a compound of formula (I) disclosed herein, or pharmaceutical compositions thereof, for use in treating, preventing or ameliorating cancer, such as a MYC-driven cancer.

Some embodiments provide for use of a compound of formula (I) disclosed herein, or pharmaceutical compositions thereof, in the manufacture of a medicament for reducing the expression or activity of MYC. In certain embodiments, a compound of formula (I) disclosed herein, or pharmaceutical compositions thereof, is used in the manufacture of a medicament to achieve one or more phenotypic outcomes such as, for example, decrease in cancer cell growth or proliferation, decrease in cancer cell viability, decrease in apoptosis, decrease in tumor volume (i.e., tumor regression), decrease in cancer metastasis, increase in animal or human subject survival, or other desired outcome with respect to particular phenotypes (e.g., body weight, metabolism, etc.). In certain embodiments, a compound of formula (I) disclosed herein, or pharmaceutical compositions thereof, is used in the manufacture of a medicament to treat, prevent or ameliorate cancer in a patient diagnosed with or susceptible to cancer, such as a MYC-driven cancer.

In other embodiments, provided herein are methods of preventing, treating, or ameliorating other diseases that are not cancer, comprising administration of a compound of formula (I) disclosed herein, or pharmaceutical compositions thereof, to a cell, tissue, animal or human subject. In particular embodiments, such diseases are associated with MYC expression or activity, wherein targeting MYC to reduce or inhibit its expression or activity can offer therapeutic benefits. Examples of such diseases include inflammatory diseases, autoimmune diseases, metabolic diseases, age-associated diseases, etc. Particular examples include asthma, renal cystic diseases (e.g., polycystic kidney disease), Crohn's disease, lipid metabolism diseases, autoimmune encephalomyelitis, etc. Madden et al. describes examples of such diseases (Madden et al., Mol. Cancer, 20, Article 3 (2021)), the disclosures of which, along with its references, are incorporated herein in their entirely.

In certain embodiments, provided herein are methods of treatment of a human subject in need thereof by administering to the human individual a therapeutically effective amount of a compound of formula (I) disclosed herein, or pharmaceutical compositions thereof, which may target one or more MYC nucleic acids disclosed herein, or part thereof. In some embodiments, administration of a therapeutically effective amount of a compound of formula (I) disclosed herein, or pharmaceutical composition thereof, is accompanied by monitoring of MYC RNA levels, protein levels, or activity, in the individual, to determine the individual's response to administration of the compound or pharmaceutical composition. A human subject's response to administration of the compound can be used by a physician to determine the dose, schedule, and duration of therapeutic intervention.

Methods of treatment comprising administration of a compound of formula (I) disclosed herein, or pharmaceutical compositions or medicaments thereof, to a cell, a tissue, an animal or a human subject can vary in terms of composition, quantity of doses, and scheduling of doses. A unit dose is a pre-determined therapeutically effective amount of a compound of formula (I) disclosed herein, or pharmaceutical compositions and medicaments thereof, that is administered. Unit doses can vary depending on various factors, including but not limited to, weight, age, gender, severity of symptoms, medical history, and aggressiveness of treatment. A schedule is the frequency of administration of unit doses. The size of a unit dose and the schedule of administration of the compounds, or pharmaceutical compositions and medicaments thereof, can be determined by a person of ordinary skill in the art and are incorporated in certain embodiments herein.

In certain embodiments, a compound of formula (I) disclosed herein, or pharmaceutical compositions or medicaments thereof, are co-administered with one or more other pharmaceutical agents. The compounds, pharmaceutical compositions, or medicaments described herein can be prepared with the one or more other pharmaceutical agents together in a single formulation or prepared separately. In some embodiments, the one or more other pharmaceutical agents are designed to treat a different disease, disorder, symptom, or condition compared to the compounds, pharmaceutical compositions, or medicaments described herein. In other embodiments, the one or more other pharmaceutical agents are designed to treat the same disease, disorder, symptom, or condition as the compounds, pharmaceutical compositions, or medicaments described herein. In some embodiments, the one or more other pharmaceutical agents are co-administered with compounds, pharmaceutical compositions, or medicaments described herein to produce an additive effect. In certain embodiments, the one or more other pharmaceutical agents are co-administered with compounds, pharmaceutical compositions or medicaments described herein to produce a synergistic or supra-additive effect, wherein the co-administration of both produces an effect that is greater than the sum of the effects of administering each alone. In other embodiments, the one or more other pharmaceutical agents are co-administered with compounds, pharmaceutical compositions, or medicaments described herein to treat undesired side effects of the compounds, pharmaceutical compositions, or medicaments described herein. In certain embodiments, compounds, pharmaceutical compositions, or medicaments described herein are co-administered with one or more other pharmaceutical agents to prevent or delay the onset of symptoms, slow disease progression, reduce side effects, improve therapeutic efficacy, or to otherwise improve patient outcomes. Co-administration, as used herein, can refer to cases where the one or more other pharmaceutical agents are administered at the same time as the compounds, pharmaceutical compositions, or medicaments described herein, as well as cases where the one or more other pharmaceutical agents are administered (e.g., a few hours, a few days, or a few weeks, etc.) prior to or after the compounds, pharmaceutical compositions, or medicaments described herein are administered.

In certain embodiments, the one or more other pharmaceutical agents that are co-administered with the compounds, pharmaceutical compositions, or medicaments described herein, can include agents comprising, for example, antisense oligonucleotides, other oligonucleotides (e.g., siRNAs, ribozymes, deoxyribozymes, or aptamers), antibodies, peptides, small molecule compounds, nucleic acid vectors, cell therapy (e.g., CAR-T), or gene therapy agents, etc. In certain embodiments, the one or more pharmaceutical agents that are co-administered with the compounds, pharmaceutical compositions, or medicaments described herein, include drugs typically administered to treat or ameliorate symptoms in cancer such as, for example, alkylating agents (e.g., altretamine, busulfan, carboplatin, chlorambucil, cisplatin, cyclophosphamide, dacarbazine, ifosfamide, melphalan, temozolomide, and trabectedin, etc.), anti-metabolites (e.g., azacitidine, capecitabine, clofarabine, cytarabine, floxuridine, fludarabine, 5-fluorouracil, gemcitabine, 6-mercaptopurine, methotrexate, pemetrexed, pentostatin, pralatrexate, trifluridine and tipiracil, etc.), anti-tumor antibiotics (e.g., bleomycin, daunorubicin, doxorubicin, doxorubicin liposomal, epirubicin, idarubicin, mitoxantrone, and valrubicin, etc.), biological response modifiers that strengthen the bodies' immune system to fight the growth of cancer or disrupt processes needed for cancer to grow or spread (e.g., Avastin, Erbitux, Herceptin, and Rituxan, etc.), corticosteroids hormones (e.g., dexamethasone, and prednisone, etc.), nitrosoureas (e.g., carmustine, and lomustine, etc.), plant alkaloids and natural products (e.g., docetaxel, etoposide, irinotecan, paclitaxel, teniposide, topotecan, vinblastine, vincristine, and vinorelbine, etc.), and sex hormones (e.g., leuprolide, and tamoxifen, etc.). In certain embodiments, the one or more pharmaceutical agents that are co-administered with the compounds, pharmaceutical compositions, or medicaments described herein, include drugs that alleviate pain, inflammation or other symptoms (e.g., COX inhibitors, phenylbutazone, ibuprofen, suprofen, fenbufen, ketoprofen, pranoprofen, carprofen, indomethacin, folinic acid, tiaprofenic acid, diclofenac, niflumic acid, diazepines or benzodiazepines (e.g., Diazepam), and barbiturate, etc.), drugs that improve uptake or delivery such as blood thinners (e.g., aspirin, and warfarin, etc.), antibacterial, antiviral, antibiotic, or other drug that provides at least one benefit, including treatment efficacy, symptom alleviation, drug tolerance, or side effect mediation, in a therapeutic setting.

In certain embodiments, one or more pharmaceutical agents that can be co-administered with compounds, pharmaceutical compositions, or medicaments described herein include an additional modulator that can reduce the expression or activity of MYC. In certain embodiments, the dose of a co-administered pharmaceutical agent is lower than the dose that would be administered if the co-administered pharmaceutical agent was administered alone.

### Methods of Development

In certain embodiments, provided herein are methods of development of small molecule compounds that are capable of targeting MYC, or its associated genes or pathways, thereby reducing the expression or activity of MYC. In certain embodiments, the methods of development of the compounds are entirely computational. In some embodiments, the methods of development of the compounds involve biochemical or cell-based methods, such as screens and selections. In some embodiments, the methods of development of the compounds involve a combination of computational methods and biochemical or cell-based methods. Such methods of development involve standard practices that are known to a person who is skilled in the art and are incorporated in certain embodiments herein.

In some embodiments, computational methods can involve artificial intelligence or machine learning software, rely on large molecular databases, utilize high throughput analysis, or any combination thereof. Computational methods described by Manigrasso et al. (Manigrasso et al., Chem, 7(11): 2965-2988 (2021)), Mendez-Lucio et al. (Mendez-Lucio et al., Nature Communications, 11, 10 (2020)), Dallakyan and Olson (Dallakyan and Olson, Hempel et al. (ed.) Chemical Biology: Methods and Protocols, Chapter 19, Methods in Molecular Biology pg 243-250 (2015)), Zoete et al. (Zoete et al., Journal of Chemical Information and Modeling, 56: 1399-1404 (2016)), and Merk et al. (Merk et al., Molecular Informatics, 37: 1700153 (2018)) are representative of some of the computational methods available, which together with references cited therein, are incorporated herein in their entirety. In some embodiments, the structure of a target RNA, such as MYC, or part thereof, is computationally predicted. Thereafter, a computational library of small molecule compounds is computationally docked individually to the target, and a computational method is used to determine the binding energy of each small molecule compound to the target. Small molecule compounds that are predicted to have favorable binding energies to the target RNA are prioritized for further analysis and development. In some embodiments, new small molecule compounds are created for computational screening from an amalgamation of existing molecules or atoms from one or more databases. Other computational methods are well known to a person of ordinary skill in the art and are included in various embodiments herein.

In certain embodiments, methods of development of small molecule compounds include high throughput biochemical or cell-based screening methods, which are known to a person of ordinary skill in the art. Physical libraries of small molecules are built or obtained from commercially available sources. These libraries are screened against a target molecule of choice, such as MYC, by introducing the small molecules to the target molecule of choice and then implementing a washing or separating method to determine binding affinity and/or specificity. An example of such a method is affinity-selection mass spectrometry (ALIS) as described by Rizvi et al. (Rizvi et al., Methods, 167: 28-38 (2019)), which along with references cited therein, are incorporated by reference in its entirety. In some embodiments, the binding of small molecule compounds to a target molecule can be detected and measured using a biophysical method such as, for example, NMR, X-ray crystallography, small-angle X-ray scattering, microscale thermophoresis, surface plasmon resonance, fluorescence-based methods, isothermal titration calorimetry, etc. In other embodiments, cell-based methods involving treatment of cells with a library of small molecule compounds, followed by detecting the levels of a target RNA (e.g., using RT-qPCR, or RNA-seq, etc.) and/or protein (e.g., using Western blot, ELISA, Alphascreen, or homogenous time-resolved fluorescence (HTRF), etc.), are used to identify compounds capable of modulating the expression or activity of a target, such as MYC. A biochemical or cell-based assay or a series of biochemical or cell-based assays can be used to determine structural and chemical properties of the small molecules or molecular complexes that are formed between the small molecule and the target molecule of choice. Controls or negative selection steps can be used to screen out small molecules with off-target binding activity to other molecules that are not the target molecule of choice. Further, screening at different concentrations of the small molecule against the target molecule of choice is used to determine other properties such as IC₅₀, EC50, potency, and the like. Further description of methods and procedures for developing small molecule compounds are described by Falese et al. (Falese et al., Chem. Soc. Rev., 50: 2224 (2021)), and Cronk (Cronk, Drug Discovery and Development (Second Edition) Chapter 8, pp. 95-117 (2013)), which along with references cited therein, are incorporated by reference in their entirety. Other biochemical or cell-based screening methods are well known to a person of ordinary skill in the art and are included in various embodiments herein.

In certain embodiments, methods of development of small molecule modulators include fragment-based discovery techniques that are known to a person of ordinary skill in the art. These methods involve screening of a library of small molecular fragments that contain one or more binding epitopes for binding affinity and/or specificity to a target molecule, such as MYC. Typically, the small molecular fragments have a molecular mass of around 120-250 Daltons. In certain cases, these fragment-based discovery methods are combined with computational methods, some of which are described below. Examples of fragment-based discovery techniques include lead identification by fragment evolution, lead identification by fragment linking, lead identification by fragment self-assembly, and lead progression by fragment optimization, which are described herein. Often, assessment of target molecule binding sites, the development of fragment complexes, and subsequent determination of efficacy or specificity of binding is informed by structural, morphological, and chemical data acquired from assessment tools such as nuclear magnetic resonance spectroscopy, mass spectrometry, or X-ray crystallography.

In lead identification by fragment evolution, a library of fragments is applied to the target molecule of choice, and the strength and specificity of binding is determined. The fragments with higher binding specificity are then reacted or evolved with various other fragments to form fragment complexes that are screened for having even higher binding specificity.

In lead identification by fragment linking, fragment libraries are screened through multiple binding sites of a target molecule of choice for binding specificity. Two or more fragments with high binding specificity to two or more nearby binding sites on a target molecule of choice are chemically linked together.

In lead identification by fragment self-assembly, also termed combinatorial chemistry, a library of fragments capable of self-assembly is introduced to a target molecule of choice. The fragments are allowed to bind to the target molecule of choice in a manner that produces a complex that inhibits the expression or activity of the target molecule of choice. The various fragments are capable of assembling together while bound to the target molecule of choice via complementary reactive groups. Once assembled, these fragment complexes can then be isolated to assess their chemical and structural properties.

In lead progression by fragment optimization, a library of fragments is used to modify the properties of an existing modulator or fragment complex. Typically, this method is used to address the optimization of certain properties, such as selectivity, solubility, stability, or efficacy.

Examples and further discussion of methods and procedures for the development of small molecule modulators via fragment-based discovery are described by Rees et al. (Rees et al., Nature Reviews Drug Discovery, 3(8): 660 (2004)), Erlanson et al. (Erlanson et al., Journal of Medicinal Chemistry, 47(14): 3463-3482 (2004)), and Congreve et al. (Congreve et al., Journal of Medicinal Chemistry, 51(13): 3661-3680 (2008)), all of which along with references cited therein, are incorporated by reference in their entirety. Other methods are well known to a skilled artisan and are included in various embodiments herein.

### Diagnostics and Kits

In some embodiments, measuring and detecting an increase in expression or activity of MYC, or an increase in signaling through a pathway associated with MYC, can be used to diagnose, or to determine an increased risk for or increased susceptibility to cancers, such as MYC-driven cancers disclosed herein. In some embodiments, measuring and detecting a decrease in expression or activity of MYC, or a decrease in signaling through a pathway associated with MYC, can be used to determine a decreased risk for or decreased susceptibility to cancers, such as MYC-driven cancers disclosed herein. In some embodiments, a method for determining a subject's susceptibility to cancers, such as MYC-driven cancers disclosed herein, comprises obtaining a sample, including from tissues, fluids, or other biological sample from a subject, detecting the expression levels of at least one MYC RNA transcript that is associated with a sequence described by SEQ ID NOs: 1-3, wherein different expression levels of the transcript(s) are associated with different susceptibilities to the disease, and determining a susceptibility to the disease. In certain embodiments, the methods of detecting the expression of MYC, activity of MYC, or signaling through a pathway associated with MYC, involves the use of a compound of formula (I) disclosed herein or conjugates thereof.

In certain embodiments, the methods of determining risk or susceptibility to a disease, or methods of diagnosis of a disease stated above, can be applied to predict prognosis of a human individual diagnosed with, or experiencing symptoms associated with, cancer, such as a MYC-driven cancer. In other embodiments, the methods of determining risk or susceptibility to a disease, or methods of diagnosis of a disease stated above, can be used to assess a human individual for a probability of a response to a therapeutic method and/or modulator used to treat, prevent or ameliorate symptoms associated with cancer, such as a MYC-driven cancer. In one embodiment, such methods can be used to select a therapeutic method or modulator used in treating a subject diagnosed with the disease.

Some embodiments also relate to kits and apparatuses for determining susceptibility of a human individual to a disease; or for diagnosing the disease; or predicting prognosis of a human individual diagnosed with, or experiencing symptoms associated with a disease; or assessing a human individual for a probability of a response to a therapeutic method and/or modulator used to treat, prevent or ameliorate symptoms associated with a disease. In some embodiments, the disease is cancer, such as a MYC-driven cancer described herein.

Kits that are useful in any of the methods described herein can comprise of any component that is useful in any of the methods described herein, including but not limited to probes (e.g., hybridization probes, allele-specific oligonucleotides), enzymes (e.g., for RFLP analysis, activity assays), reagents for amplification of nucleic acids, reagents for direct analysis of at least one allele of at least one polymorphic marker within or associated with MYC, reagents for indirect analysis of at least one allele of at least one polymorphic marker within or associated with MYC, reagents for detecting the expression or activity of MYC, reagents for detecting signaling through a pathway associated with MYC, etc. In one embodiment, the kit can include necessary buffers. In another embodiment, the kit can additionally provide reagents for other disease-specific diagnostic methods known in the art to be carried out in conjunction with the methods described herein.

In certain embodiments, the reagents in the kits include at least one compound of formula (I) disclosed herein, or complexes or conjugates thereof, which is capable of interacting with MYC, or otherwise to detect the expression of MYC, activity of MYC, or signaling through a pathway associated with MYC. In another embodiment, the kit comprises at least one or more labeled compounds of formula (I) disclosed herein, and reagents for detection of the label. Suitable labels can include but are not limited to a radioisotope, a fluorescent label, an enzyme label, an enzyme co-factor label, a magnetic label, a spin label, or an epitope label, etc.

In certain embodiments, the kits and apparatuses include a look-up table comprising correlation data between the expression levels of at least one transcript associated with a sequence described by SEQ ID NOs: 1-3 that is selectively assessed by the kit, and susceptibility to a disease, or prognosis for the disease, or response to at least one therapy for the disease. Another set of embodiments relates to methods of use of a compound of formula (I) disclosed herein in the manufacture of a reagent for diagnosing or assessing the susceptibility to a disease, or prognosis for the disease, or response to a therapy for the disease, in a human individual. In one embodiment, the kit further comprises a set of instructions for using the reagents comprising the kit. In another embodiment, the kit comprises a set of instructions or guidelines for interpreting the results of a test using the reagents comprising the kit.

A further set of embodiments provides for a kit (also referred to as a pharmaceutical pack and are used interchangeably) comprising a compound of formula (I) disclosed herein, or pharmaceutical compositions and medicaments thereof, and a set of instructions for administration of the compound, or pharmaceutical compositions and medicaments thereof, to a human. In some embodiments, an individual identified as a carrier of at least one allele of at least one polymorphic marker associated with MYC-driven cancer, is instructed to take a prescribed dose of a compound of formula (I) disclosed herein, or pharmaceutical compositions and medicaments thereof. In other embodiments, an individual with increased risk for or increased susceptibility to cancers, such as MYC-driven cancers, is instructed to take a prescribed dose of a compound of formula (I) disclosed herein, or pharmaceutical compositions and medicaments thereof. In other embodiments, an individual diagnosed with a cancer, such as a MYC-driven cancer, is instructed to take a prescribed dose of a compound of formula (I) disclosed herein, or pharmaceutical compositions and medicaments thereof.

### Computers-Readable Medium and Apparatuses

The compositions, methods, and kits described herein can be implemented, in all or in part, as computer executable instructions on computer-readable media. As understood by a person skilled in the art, the various steps of the compositions, methods and kits described herein can be implemented as various blocks, operations, routines, tools, modules and techniques, which in turn can be implemented in hardware, firmware, software, or any combination of hardware, firmware, and/or software. In certain embodiments, hardware implementations can include but are not limited to a custom integrated circuit (IC), an application specific integrated circuit (ASIC), a field programmable logic array (FPGA), a programmable logic array (PLA), etc. In other embodiments, when implemented as software, the software can be stored in any computer readable medium known in the art, including but not limited to a solid-state disk, a magnetic disk, an optical disk, or other storage medium, in a RAM or ROM or flash memory of a computer, processor, hard disk drive, thumb drive, optical disk drive, tape drive, etc. In one embodiment, the software can be delivered to a user or a computing system via any delivery method known in the art, including but not limited to over a communication channel such as the internet, a wireless connection, a satellite connection, a telephone line, a computer readable disk or other transportable computer storage mechanism.

One set of embodiments provides for a suitable computing system environment known in the art to implement the compositions, methods and kits described herein, including but not limited to mobile phones, laptops, personal computers, server computers, multiprocessor systems, microprocessor-based systems, set top boxes, programmable consumer electronics, network PCs, minicomputers, mainframe computers, cloud computing environments, and distributed computing environments that include any of the above systems or devices, etc. In some embodiments, the steps of the compositions, methods or kits described herein are implemented via computer-executable instructions such as program modules, including but not limited to routines, programs, objects, components, data structures, etc. that perform particular tasks or implement particular abstract data types. In one embodiment, the methods and apparatuses are practiced in a distributed computing environment, where tasks are performed by remote processing devices that are linked through a communications network. In one embodiment, the methods and apparatuses are practiced in an integrated computing environment. In both integrated and distributed computing environments, program modules can be located in both local and/or remote computer storage media, including memory storage devices.

Thus, one set of embodiments provides a computer-readable medium having computer executable instructions for determining the effect of administering a compound of formula (I) disclosed herein or pharmaceutical compositions and medicaments thereof, to a cell an animal, or a human subject, the computer-readable medium comprising data indicative of the level of at least one protein, RNA, biomarker, or other phenotype, and a routine stored on the computer readable medium and adapted to be executed by a processor to determine the effect of administering the compound or compositions thereof from the data. In certain embodiments, the effect being determined is a change in levels of MYC RNA, or a change in levels of MYC protein, or a change in one or more phenotypes such as, for example, cell growth or proliferation, cell viability, cell morphology, cell cycle arrest, cell migration, cell invasion, apoptosis, survival, cancer cell growth, cancer metastasis, behavior, body weight, metabolism, etc. In one embodiment, the computer-readable medium is used to determine progression of a disease such as cancer, and its response to administration of a compound of formula (I) disclosed herein or pharmaceutical compositions and medicaments thereof to a human subject.

Another set of embodiments provides for a computer-readable medium having computer executable instructions for developing a compound of formula (I) disclosed herein, using at least one computational method described herein, or other computational methods that are known to those skilled in the art, which are also included in the embodiments herein. The computer-readable medium can comprise data associated with a particular nucleotide sequence, SEQ ID NO, or portion thereof disclosed herein, as well as any resulting polypeptide sequences due to transcription and translation of said nucleotide sequences. The computer-readable medium can also be adapted to be executed by a processor to develop a compound of formula (I) disclosed herein from said data.

Many modifications and variations can be made in the compositions, methods, and kits described herein without departing from the spirit and scope of the invention. Accordingly, it should be understood that the compositions, methods, and kits described herein are illustrative only and are not limiting upon the scope of the invention.

### EXAMPLES

The following examples are put forth so as to provide those of ordinary skill in the art with a complete disclosure and description of how to make and use the present invention, and are not intended to limit the scope of what the inventors regard as their invention, nor are the examples intended to represent or imply that the experiments below are all of or the only experiments performed. It will be appreciated by persons skilled in the art that numerous variations and/or modifications can be made to the invention as shown in the specific embodiments without departing from the spirit or scope of the invention as broadly described. It should also be appreciated that the examples provide enabling guidance on the use of the combined features of the disclosure to apply such compositions, methods and systems to other uses. The present embodiments are, therefore, to be considered in all respects as illustrative and not restrictive.

The examples can be implemented in certain embodiments by computers or other processing devices incorporating and/or running software, where the methods and features, software, and processors utilize specialized methods to analyze data.

Efforts have been made to ensure accuracy with respect to numbers used (e.g., amounts, intensity, temperature, etc.) but some experimental errors and deviations should be accounted for.

### Example 1: Modulation of MYC Protein Expression by Test Compounds

The MYC homogenous time-resolved fluorescence (HTRF) assay kit (CisBio/Perkin Elmer 63ADK053PEG) is used to quantify the level of MYC protein in human cell lines (e.g., A549, HepG2) following treatment with test compounds, according to the following protocol. Human cells cultured in DMEM-10% FBS media are seeded at 20,000 cells per well in a 96-well plate. After 1 day, the cells are treated with test compounds at a final concentration of about 20 μM. After a 2-day incubation period, the supernatant is removed from the wells and the HTRF assay is carried out according to the kit manufacturer's protocol. In brief, lysis buffer #4 is added to create cell lysates. 10 μL of cell lysate is added to 10 μL of premixed antibody solution (anti-human My c-Eu cryptate antibody and anti-human Myc-d2 antibody) prepared in detection buffer #3 in a 384-well low-volume white-walled plated. After 3 h of incubation, fluorescence readings are taken at the emission wavelengths of donor and acceptor fluorophores (620 nm and 665 nm respectively) using a HTRF-compatible plate reader. The ratio of acceptor and donor fluorescence emission signals for each sample is calculated by dividing the fluorescence signal at 665 nm by the signal at 620 nm, and further normalized by dividing with the concentration of total protein in that sample as determined by the BCA assay. The values so obtained are presented as a percentage of that of the no-compound, solvent only (DMSO)-treated samples. A lower percentage of MYC protein in a sample with respect to the DMSO-treated sample indicates greater percentage reduction in MYC protein level due to the test compound, and vice versa.

Table 1 provides the percentage reduction in MYC protein levels due to particular test compounds when tested using the HTRF assay protocol described above. Particular compounds of formula (I) disclosed herein reduce MYC protein levels by more than 10%, 20%, 30%, 40%, 50%, 60%, or 70%, compared to the solvent only (DMSO)-treated sample.

The level of reduction of MYC protein following treatment with test compounds is also assessed by Western blot according to the following protocol. The human cancer cell lines (e.g., A549 or HepG2) are seeded at 20,000 cells per well in a 96-well plate. After 1 day, the cells are treated with test compounds at a final concentration of about 1 μM. After a 2-day incubation period, the supernatant is removed from the wells and RIPA buffer is added to create cell lysates. The lysates are analyzed by Western blot and probed with a MYC-specific antibody (e.g., Proteintech 10828-1-AP) together with a GAPDH-specific antibody (e.g., Proteintech 10494-1-AP) to serve as loading control. The intensities of Western blot bands are quantified by FIJI (Image J). The relative expression value of MYC following treatment with a test compound is calculated by dividing the intensity of the MYC band of the sample with the intensity of its own GAPDH band. The value so obtained is presented as a percentage of that of the no-compound, solvent only (DMSO)-treated sample. A lower percentage of MYC protein in a sample with respect to the DMSO-treated sample indicates greater percentage reduction in MYC protein level due to the test compound, and vice versa.

Table 2 provides the percentage reduction in MYC protein levels due to particular test compounds when tested using the Western blot assay described above. Particular compounds of formula (I) disclosed herein reduce MYC protein levels by more than 10%, 20%, 30%, 40%, 50%, or 60%, compared to the solvent only (DMSO)-treated sample.

### Example 2: Modulation of MYC RNA Expression by Test Compounds

The level of reduction of MYC RNA following treatment with test compounds is assessed using RT-qPCR according to the following protocol. The human cancer cell lines (e.g., A549, HepG2) are seeded at 20,000 cells per well in a 96-well plate. After 1 day, the cells are treated with test compounds at a final concentration of about 1 μM. After a 1-day incubation period, the supernatant is removed from the wells and lysis solution from the Cells-to-CT 1-Step Power SYBR Green Kit (Invitrogen A25599) is added to create cell lysates, according to the kit manufacturer's protocol. The kit manufacturer's protocol is then followed to perform RT-qPCR for each sample using a pair of MYC-specific primers (e.g., SEQ ID NO: 4 and SEQ ID NO: 5) to detect MYC RNA, as well as a pair of beta-actin-specific primers (e.g., SEQ ID NO: 6 and SEQ ID NO: 7) to detect beta-actin RNA, which serves as a loading control. The relative expression levels of MYC and beta-actin RNAs are calculated using a standard curve generated from a dilution series of untreated cell lysates. The normalized level of MYC RNA following treatment with a test compound is calculated by dividing the relative expression level of MYC in the sample by the relative expression level of beta actin in the same sample. The value so obtained is presented as a percentage of that of the no-compound, solvent only (DMSO)-treated sample. A lower percentage of MYC RNA in a sample with respect to the DMSO-treated sample indicates greater percentage reduction in MYC RNA levels due to the test compound, and vice versa.

Table 3 provides the percentage reduction in MYC RNA levels due to particular test compounds when tested using the RT-qPCR assay described above. Particular compounds of formula (I) disclosed herein reduce MYC RNA levels by more than 10%, 20%, 30%, or 40% compared to the solvent only (DMSO)-treated sample.

### Example 3: Effects of Test Compounds on Cell Viability Determined Using CCK-8 Assay

The effect of test compounds on cell viability is determined using the CCK-8 assay kit (Dojindo CK04-1000) according to the following protocol. The human cancer cell lines (e.g., A549, HepG2, JeKo-1, MDA-MB-231, etc.) and non-cancer human cell lines (e.g., HEK-293, HaCaT, etc.), are seeded at 20,000 cells per well in a 96-well plate. After 1 day, the cells are treated with test compounds at various final concentrations (e.g., 10 nM, 31.6 nM, 100 nM, 316 nM, 1 μM, 3.16 μM, 10 μM, 20 μM, etc.). After a 2-day or 3-day incubation period, 10 μL of CCK-8 solution, which contains the WST-8 dye, is added to each well according to the kit manufacturer's protocol. After incubation for 1-4 hours, the absorbance at 450 nm is measured using a microplate reader. The absorbance at 450 nm measures the amount of WST-8 that is reduced to WST-8 formazan by cellular dehydrogenases, which is directly proportional to the number of living cells. The cytotoxic effect of test compounds at different concentrations is calculated by subtracting from 1 the ratio of absorbance of the compound-treated sample at 450 nm to that of the no-compound, solvent only (DMSO)-treated sample. A higher number indicates a greater cytotoxic effect of the test compound on cells (corresponding to a greater reduction in cellular dehydrogenase activity), and vice versa. The IC₅₀ values for the cytotoxic effect of each test compound in A549, HepG2, JeKo-1, and/or MDA-MB-231 cells are determined by plotting a dose-response curve to calculate the concentration of test compound that produces half of the maximum cytotoxic effect. The cytotoxic effect and IC₅₀ of test compounds are also measured in a non-cancer human cell line (e.g., HEK-293, and/or HaCaT) as a proxy for healthy cells to determine the therapeutic window of the test compounds (i.e., the concentration range at which the test compounds selectively kill cancer cells but not healthy cells). The therapeutic index of a test compound is defined herein as its IC₅₀ in HEK-293 cells divided by its IC₅₀ in HepG2 cells. The higher the therapeutic index, the safer and more useful the test compounds are as cancer drugs, as they exhibit greater selectively in killing cancer cells but not healthy cells.

Table 4 provides the IC₅₀ of particular test compounds when tested using the CCK-8 assay described above. Particular compounds of formula (I) exhibit an IC₅₀ of less than 10 μM, less than 1 μM, or less than 0.5 μM when tested using the CCK-8 assay described above. Particular compounds of formula (I) exhibit a therapeutic index of more than 1, more than 10, or more than 100, when tested using the CCK-8 assay described above. As a benchmark, one of the best-known inhibitors of MYC, sAJM589, which acts to inhibit the MYC-MAX protein-protein interaction, exhibits an IC₅₀ of 28.4 μM, 20.5 μM and 34.2 μM (therapeutic index of 1.67) when tested in A549, HepG2, and HEK-293 cells respectively. Thus, the CCK-8 assay results show that one or more compounds of formula (I) disclosed herein are more potent at killing cancer cells compared to sAJM589. In addition, one or more compounds of formula (I) disclosed herein have a higher therapeutic index (and thus would be a safer drug) compared to sAJM589.

### Example 4: Effects of Test Compounds on Cell Viability Determined Using CellTiter-Glo 2.0 Assay

The effect of test compounds on cell viability is also determined using the CellTiter-Glo 2.0 assay kit (Promega G9241) according to the following protocol. The human cancer cell line (e.g., HepG2) and a non-cancer human cell line (e.g., HEK-293), are seeded at 20,000 cells per well in a 96-well plate. After 1 day, the cells are treated with test compounds at various concentrations (e.g., 10 nM, 31.6 nM, 100 nM, 316 nM, 1 μM, 3.16 μM, 10 μM, 20 μM, etc.). After a 2-day incubation period, the CellTiter-Glo 2.0 assay is performed according to the kit manufacturer's protocol. In brief, an equal volume of CellTiter-Glo 2.0 Reagent is added to each well (e.g., 100 μL of CellTiter-Glo 2.0 Reagent is added to 100 μL of medium containing cells). The contents are mixed for 2 minutes on an orbital shaker to induce cell lysis, and incubated for 10 minutes at room temperature, following which the luminescent signal is measured using a microplate reader. The intensity of the luminescent signal is directly proportional to the amount of ATP present, which in turn is directly proportional to the number of metabolically active cells. The cytotoxic effect of test compounds at different concentrations is calculated by subtracting from 1 the ratio of the luminescence signal of the compound-treated sample to that of the no-compound, solvent only (DMSO)-treated sample. A higher value indicates a greater cytotoxic effect of the test compound on cells (corresponding to a greater reduction in metabolically active cells), and vice versa. The IC₅₀ values for the cytotoxic effect of each test compound in HepG2 cells are determined by plotting a dose-response curve to calculate the concentration of test compound that produces half of the maximum cytotoxic effect. The cytotoxic effect and IC₅₀ of test compounds are also measured in a non-cancer human cell line (e.g., HEK-293) as a proxy for healthy cells to determine the therapeutic window of the test compounds (i.e., the concentration range at which the test compounds selectively kill cancer cells but not healthy cells). The therapeutic index of a test compound is defined herein as its IC₅₀ in HEK-293 cells divided by its IC₅₀ in HepG2 cells. The higher the therapeutic index, the safer and more useful the test compounds are as cancer drugs in selectively killing cancer cells yet minimizing side effects on healthy cells.

Table 5 provides the IC₅₀ and therapeutic index of particular test compounds when tested using the CellTiter-Glo 2.0 assay described above. Particular compounds of formula (I) exhibit an IC₅₀ of less than 10 μM, less than 1 μM, or less than 0.2 μM when tested using the CellTiter-Glo 2.0 assay described above. Particular compounds of formula (I) exhibit a therapeutic index of more than 1, more than 10, or more than 100, when tested using the CellTiter-Glo 2.0 assay described above. In comparison, one of the best-known inhibitors of MYC, sAJM589, which acts to inhibit the MYC-MAX protein-protein interaction, exhibits an IC₅₀ of 13 μM and 12.2 μM (therapeutic index of 0.94) when tested in HepG2 and HEK-293 cells respectively. In addition, one of the best-known cancer drugs, paclitaxel (Taxol), which kills dividing cells by interfering with the normal function of microtubule growth, exhibits an IC₅₀ of 7.5 μM and 27 μM (therapeutic index of 3.6) when tested in HepG2 and HEK-293 cells respectively. Thus, the CellTiter-Glo 2.0 assay shows that one or more compounds of formula (I) disclosed herein are more potent at killing cancer cells compared to sAJM589. In addition, one or more compounds of formula (I) disclosed herein have a higher therapeutic index (and thus would be a safer drug) compared to both sAJM589 and Taxol.

### Example 5: Effects of Test Compounds on Cell Viability and Apoptosis Determined Using ApoTox-Glo Triplex Assay

The effects of test compounds on cell viability and apoptosis are determined using the ApoTox-Glo Triplex assay kit (Promega G6320) according to the following protocol. The human cancer cell line (e.g., HepG2) and a non-cancer human cell line (e.g., HEK-293), are seeded at 20,000 cells per well in a 96-well plate. After 1 day, the cells are treated with test compounds at various final concentrations (e.g., 10 nM, 31.6 nM, 100 nM, 316 nM, 1 μM, 3.16 μM, 10 μM, 20 μM, etc.). After a 2-day incubation period, the ApoTox-Glo Triplex assay is performed according to the kit manufacturer's protocol. In brief, 20 μL of viability/cytotoxicity reagent containing both GF-AFC substrate (cell-permeant substrate to measure live-cell protease activity) and bis-AAF-R110 substrate (cell-impermeant substrate to measure dead-cell protease activity) is added to each sample, and briefly mixed by orbital shaking (300-500 rpm for ˜30 seconds). After incubation for 30 minutes at 37° C., the fluorescence signal is measured at two wavelength sets (400_{Ex}/505_{Em}) and (485_{Ex}/520_{Em}) to assess live-cell protease activity and dead-cell protease activity respectively. Following the measurement of the live-cell and dead-cell protease activities, 25 μl of Caspase-Glo 3/7 Reagent containing a luminogenic DEVD-peptide substrate for caspase-3/7 and thermostable luciferase is added to each sample, and briefly mixed by orbital shaking (1300-1500 rpm for ˜30 seconds). After incubation for 30 minutes at room temperature, the luminescent signal is measured using a microplate reader. The luminescent signal directly correlates with caspase-3/7 activity, which is a key indicator of apoptosis.

The cytotoxic effect of test compounds according to the ApoTox-Glo Triplex assay is calculated as follows. The live-dead ratio is calculated by dividing the fluorescence signal for live-cell protease activity with the fluorescence signal for dead-cell protease activity. The cytotoxic effect of test compounds at different concentrations is calculated by dividing the live-dead ratio of the compound-treated sample with that of the no-compound, solvent only (DMSO)-treated sample, and subtracting the result from 1. A higher value indicates a greater cytotoxic effect of the test compound on cells (corresponding to a lower live-dead ratio as assessed by live-cell and dead-cell protease activity), and vice versa. The IC₅₀ values for the cytotoxic effect of each test compound in HepG2 cells are determined by plotting a dose-response curve to calculate the concentration of test compound that produces half of the maximum cytotoxic effect. The cytotoxic effect and IC₅₀ of test compounds are also measured in a non-cancer human cell line (e.g., HEK-293) as a proxy for healthy cells to determine the therapeutic window of the test compounds (i.e., the concentration range at which the test compounds selectively kill cancer cells but not healthy cells). The therapeutic index of a test compound is defined herein as its IC₅₀ in HEK-293 cells divided by its IC₅₀ in HepG2 cells. The higher the therapeutic index, the safer and more useful the test compounds are as cancer drugs in selectively killing cancer cells yet minimizing side effects on healthy cells.

Table 6 provides the IC₅₀ and therapeutic index of particular test compounds when tested using the ApoTox-Glo Triplex assay described above. Particular compounds of formula (I) exhibit an IC₅₀ of less than 10 μM, less than 1 μM, or less than 0.2 μM when tested using the ApoTox-Glo Triplex assay described above. Particular compounds of formula (I) exhibit a therapeutic index of more than 1, more than 10, or more than 100, when tested using the ApoTox-Glo Triplex assay described above. In comparison, one of the best-known inhibitors of MYC, sAJM589, which acts to inhibit the MYC-MAX protein-protein interaction, exhibits an IC₅₀ of 3 μM and 13.6 μM (therapeutic index of 4.53) when tested in HepG2 and HEK-293 cells respectively. In addition, one of the best-known cancer drugs, paclitaxel (Taxol), which kills dividing cells by interfering with the normal function of microtubule growth, exhibits an IC₅₀ of 2.6 μM and 6.9 μM (therapeutic index of 2.65) when tested in HepG2 and HEK-293 cells respectively. Thus, the ApoTox-Glo Triplex assay shows that one or more compounds of formula (T) disclosed herein are more potent at killing cancer cells compared to sAJM589. In addition, one or more compounds of formula (I) disclosed herein have a higher therapeutic index (and thus would be a safer drug) compared to both sAJM589 and Taxol.

The results of the ApoTox-Glo Triplex assay show an increase in fold-change of caspase 3/7 activation in HepG2 cells with increasing concentrations of sAJM589 and Taxol, indicating that the cytotoxic effects of sAJM589 and Taxol are mediated via the apoptotic pathway. In contrast, increasing concentrations of a compound of formula (I) disclosed herein leads to a decrease in fold-change of caspase 3/7 activation in HepG2 cells, indicating that the cytotoxic effect of the compound is mediated through a non-apoptotic pathway. These results are consistent with the effect of compounds of formula (I) disclosed herein in reducing the level of MYC protein (see Example 1), as MYC is known to upregulate caspase activity and MYC inhibition has been shown to lead to non-apoptotic cancer cell death and growth arrest (Fornari et al., Biochem. Pharmacol., 51(7): 931-940 (1996)). The fold-change of caspase 3/7 activation is calculated by dividing the luminescence signal of the compound-treated sample with that of the no-compound, solvent only (DMSO)-treated sample. The fold-change of caspase 3/7 activation following treatment of HepG2 cells with different concentrations of test compounds are reported in Table 7.

### Example 6: In Vivo Efficacy of Test Compounds on Cancer Cells Determined Using the Mouse Hollow Fiber Model

The in vivo efficacy of test compounds (e.g., 6-(2,4-dimethylthiazol-5-yl)-2-((1-(pyrido[2,3-d]pyrimidin-4-yl)piperidin-4-yl)methyl)pyridazin-3(2H)-one) on cancer cells is determined using the mouse hollow fiber model established at the National Cancer Institute (NCI). Triple negative breast cancer (TNBC) (MDA-MB-231), pancreatic cancer (Mia-PaCa-2), and liver cancer (HepG2) cells are added to differently colored hollow fibers (KrosoFlo@Implant membrane, Green: Lot No.: 20051617, Blue: Lot No.: 20008858, White: Lot No.: 20051615) and sealed at both ends. Three hollow fibers each containing a different cancer line are surgically implanted subcutaneously into both the left and right flanks of mice (total of six hollow fibers per mouse). Drug treatment is initiated on Day 0, three days post implantation of fibers. Each treatment group has 2 mice (4 hollow fibers per cancer type): Group 1: Vehicle control; Group 2: Gemcitabine control dosed intraperitoneally (i.p.) at 50 mg/kg once every three days; Group 3: Test compound dosed orally at 50 mg/kg once daily for five days. All the animals are euthanized at the end of treatment on Day 5 with an overdose of CO₂. The fibers are excised from the mice and a modified MTT assay is used to determine cytotoxicity of the compounds on the cells in the hollow fibers. Mean % net cell growth is calculated using the formula [(mean OD(day5)−mean OD(day0))/(mean OD(day0))×100%]. Mean % inhibition of cell growth due to treatment is calculated relative to the mean % net cell growth of the Day 5 vehicle control for the particular cancer line.

The results show that oral administration of 50 mg/kg of test compound once daily for five days effectively inhibits the growth of MYC-driven cancers such as TNBC, pancreatic and liver cancer in vivo. The test compound showed the highest efficacy for growth inhibition of TNBC (88.2%), followed by pancreatic cancer (63.0%) and liver cancer (28.1%). This study shows that the test compound is effective in vivo against metastatic TNBC against which standard-of-care drugs are ineffective (MDA-MB-231 is derived from a patient with metastatic TNBC and commonly used to model late-stage breast cancer). In follow-up studies motivated by the large difference in HepG2 efficacy in vitro vs in vivo, it was found that the efficacy of the test compound against the 3 cancers in the hollow fiber model is underestimated as the test compound interacts with the MTT assay used to assess efficacy in the hollow fiber study. In direct comparison with orthogonal cell viability assays (CCK-8, Trypan Blue, and CellTiter-Glo), MTT underestimates the potency of the test compound against MDA-MB-231, Mia PaCa-2 and HepG2 by 2×, 3× and 21× respectively. Thus, the true efficacy of the test compound for growth inhibition of TNBC, pancreatic and liver cancer using the mouse hollow fiber model is projected to be >90% for all 3 cancers.

Once daily oral administration of 50 mg/kg of test compound produces no significant changes in body weight versus the vehicle-treated group, nor any signs of toxicity in treated mice according to gastrointestinal (e.g., distension of abdomen, diarrhea, soft stool, bloody stool, constipation) and general clinical signs (e.g., lethargy, emaciation, salivation, hunched back, restlessness, dehydration, head tilt, recumbency). This provides early data confirming the safety of the test compound when administered at therapeutically effective doses once daily for 5 days.

### Example 7: In Vivo Efficacy of Test Compounds on Cancer Cells Determined Using a Mouse Xenograft Model of TNBC

The in vivo efficacy of test compounds (e.g., 6-(2,4-dimethylthiazol-5-yl)-2-((1-(pyrido[2,3-d]pyrimidin-4-yl)piperidin-4-yl)methyl)pyridazin-3(2H)-one) on cancer cells is determined using a mouse xenograft model of TNBC (MDA-MB-231). 5×10⁶ MDA-MB-231 cells (in 0.2 ml of FBS-free medium containing 50% Matrigel) are injected into the subcutaneous tissue of each mouse. Treatment is initiated (Day 0) when the average tumor volume reaches 100 mm³. Tumor bearing mice are treated with vehicle and different doses of test compound (15 mg/kg, 35 mg/kg, 50 mg/kg, and 100 mg/kg) orally, once daily, for 3 weeks. Tumor volumes (mm³) are measured post-implantation of cells once every 2-3 days throughout the study. At the end of the treatment period, a portion of the tumor tissue is excised and homogenized to analyze the levels of MYC biomarker using ELISA. Blood samples are collected from mice treated with test compound 8 hours after the final dose.

The results show that treatment with the test compound leads to dose-dependent decreases in mean tumor volumes when compared to the vehicle control on day 21 (Table 8). This correlates with dose-dependent increases in concentrations of test compound in the plasma and dose-dependent decreases in MYC biomarker levels in the tumor following treatment with the test compound (Table 9). Histopathology of tumors shows that treatment with the test compound leads to a dose-dependent decrease in tumor cells, increase in necrosis and decrease in blood vessel formation compared to the vehicle. The reduction in tumor cell population and blood vessels in the treatment groups compared with vehicle control is suggestive of tumor regression. Body weight and clinical signs of animals are apparently normal throughout the study, confirming the safety of the test compound when administered at therapeutically effective doses once daily for 3 weeks.

### Tables

TABLE 1
Percentage reduction of MYC protein
by test compounds using HTRF assay

| | % reduction |
|---|---|
| | of MYC |
| Compound name | protein |
| 6-(2,4-dimethylthiazol-5-yl)-2-((1-(pyrido[2,3-d]pyrimidin- | 72 |
| 4-yl)piperidin-4-yl)methyl)pyridazin-3(2H)-one | |
| 2-((1-(7-fluoroquinazolin-4-yl)piperidin-4-yl)methyl)-6- | 74 |
| (1H-pyrazol-1-yl)pyridazin-3(2H)-one | |
| 6-(2,4-dimethylthiazol-5-yl)-2-((1-(6-methylpyrimidin-4- | 19 |
| yl)piperidin-4-yl)methyl)pyridazin-3(2H)-one | |
| 6-(2,4-dimethylthiazol-5-yl)-2-((1-(2-methylpyrido[3,4- | 16 |
| d]pyrimidin-4-yl)piperidin-4-yl)methyl)pyridazin-3(2H)-one | |
| 2-((1-(9H-purin-6-yl)piperidin-4-yl)methyl)-6-(2,4- | 17 |
| dimethylthiazol-5-yl)pyridazin-3(2H)-one | |

TABLE 2
Percentage reduction of MYC protein
by test compounds using Western blot

| | % reduction |
|---|---|
| | of MYC |
| Compound name | protein |
| 6-(2,4-dimethylthiazol-5-yl)-2-((1-(pyrido[2,3-d]pyrimidin- | 67 |
| 4-yl)piperidin-4-yl)methyl)pyridazin-3(2H)-one | |
| 2-(1-(1,6-dimethyl-1H-pyrazolo[3,4-d]pyrimidin-4- | 19 |
| yl)azetidin-3-yl)-6-(pyridin-4-yl)pyridazin-3(2H)-one | |
| 6-(1H-pyrazol-1-yl)-2-((1-(3-(trifluoromethyl)- | 33 |
| [1,2,4]triazolo[4,3-b]pyridazin-6-yl)piperidin-4- | |
| yl)methyl)pyridazin-3(2H)-one | |
| 6-(1H-1,2,4-triazol-1-yl)-2-((1-(3-(trifluoromethyl)- | 29 |
| [1,2,4]triazolo[4,3-b]pyridazin-6-yl)piperidin-4- | |
| yl)methyl)pyridazin-3(2H)-one | |
| 6-(3,5-dimethyl-1H-pyrazol-1-yl)-2-((1-(3-methyl- | 48 |
| [1,2,4]triazolo[4,3-b]pyridazin-6-yl)azetidin-3- | |
| yl)methyl)pyridazin-3(2H)-one | |
| 6-(3,5-dimethyl-1H-pyrazol-1-yl)-2-(1-(3-methyl-3H- | 24 |
| imidazo[4,5-b]pyridin-2-yl)piperidin-4-yl)pyridazin- | |
| 3(2H)-one | |

TABLE 3
Percentage reduction of MYC RNA
by test compounds using RT-qPCR

| | % reduction |
|---|---|
| | of MYC |
| Compound name | RNA |
| 6-(2,4-dimethylthiazol-5-yl)-2-((1-(pyrido[2,3-d]pyrimidin- | 45 |
| 4-yl)piperidin-4-yl)mcthyl)pyridazin-3(2H)-onc | |
| 2-((1-(7-fluoroquinazolin-4-yl)piperidin-4-yl)methyl)-6- | 12 |
| (1H-pyrazol-1-yl)pyridazin-3(2H)-one | |
| 6-(2,4-dimethylthiazol-5-yl)-2-((1-(6-methylpyrimidin-4- | 19 |
| yl)piperidin-4-yl)methyl)pyridazin-3(2H)-one | |
| 6-(2,4-dimethylthiazol-5-yl)-2-((1-(2-methylpyrido[3,4- | 5 |
| d]pyrimidin-4-yl)piperidin-4-yl)methyl)pyridazin-3(2H)-one | |
| 2-((1-(9H-purin-6-yl)piperidin-4-yl)methyl)-6-(2,4- | 1 |
| dimethylthiazol-5-yl)pyridazin-3(2H)-one | |
| 6-(1H-pyrazol-1-yl)-2-((1-(3-(trifluoromethyl)- | 19 |
| [1,2,4]triazolo[4,3-b]pyridazin-6-yl)piperidin-4- | |
| yl)methyl)pyridazin-3(2H)-one | |
| 6-(1H-1,2,4-triazol-1-yl)-2-((1-(3-(trifluoromethyl)- | 20 |
| [1,2,4]triazolo[4,3-b]pyridazin-6-yl)piperidin-4- | |
| yl)methyl)pyridazin-3(2H)-one | |
| 6-(3,5-dimethyl-1H-pyrazol-1-yl)-2-((1-(3-methyl- | 4 |
| [1,2,4]triazolo[4,3-b]pyridazin-6-yl)azetidin-3- | |
| yl)methyl)pyridazin-3(2H)-one | |

TABLE 4
IC₅₀ and therapeutic index of test compounds using the CCK-8 assay

| | Cell | IC₅₀ | Therapeutic |
|---|---|---|---|
| Compound name | line | (μM) | Index |
| 6-(2,4-dimethylthiazol-5-yl)-2-((1- | A549 | 1.3 | Very high (as |
| (pyrido[2,3-d]pyrimidin-4-yl)piperidin-4- | HepG2 | 0.3 | compound is |
| yl)methyl)pyridazin-3(2H)-one | HEK-293 | Not toxic at | not toxic to |
| | | concentrations | HEK-293 at |
| | | tested | concentrations |
| | JeKo-1 | 0.4 | tested) |
| | MDA-MB-231 | 0.8 | |
| 6-(1H-pyrazol-1-yl)-2-((1-(3- | HepG2 | 18.5 | Not |
| (trifluoromethyl)-[1,2,4]triazolo[4,3- | | | determined |
| b]pyridazin-6-yl)piperidin-4- | | | |
| yl)methyl)pyridazin-3(2H)-one | | | |
| 6-(1H-1,2,4-triazol-1-yl)-2-((1-(3- | HepG2 | 5.7 | Not |
| (trifluoromethyl)-[1,2,4]triazolo[4,3- | | | determined |
| b]pyridazin-6-yl)piperidin-4- | | | |
| yl)methyl)pyridazin-3(2H)-one | | | |
| 2-((1-(9H-purin-6-yl)azetidin-3- | HepG2 | 6.0 | Not |
| yl)methyl)-6-(pyridin-4-yl)pyridazin- | | | determined |
| 3(2H)-one | | | |
| 2-((1-(6-methylpyrazin-2-yl)piperidin-4- | HepG2 | 11.2 | Not |
| yl)methyl)-6-(1H-1,2,4-triazol-1- | | | determined |
| yl)pyridazin-3(2H)-one | | | |
| 6-(3,5-dimethyl-1H-pyrazol-1-yl)-2-(1-(3- | HepG2 | 5.3 | Not |
| methyl-3H-imidazo[4,5-b]pyridin-2- | | | determined |
| yl)piperidin-4-yl)pyridazin-3(2H)-one | | | |
| 6-(2,4-dimethyl-1,3-thiazol-5-yl)-2-(3,3- | A549 | 1.8 | Not |
| difluoro-1-pyrido[2,3-d]pyrimidin-4- | HepG2 | 0.3 | determined |
| ylpiperidin-4-yl)methylpyridazin-3-one | JeKo-1 | 0.2 | |
| | MDA-MB-231 | 1.0 | |
| 6-(2,4-dimethyl-1,3-thiazol-5-yl)-2-(3- | HepG2 | 0.5 | Not |
| fluoro-1-pyrido[2,3-d]pyrimidin-4- | JeKo-1 | 0.3 | determined |
| ylpiperidin-4-yl)methylpyridazin-3-one | MDA-MB-231 | 1.1 | |

TABLE 5
IC₅₀ and therapeutic index of test compounds
using the CellTiter-Glo 2.0 assay

| | Cell | IC₅₀ | Therapeutic |
|---|---|---|---|
| Compound name | line | (μM) | Index |
| 6-(2,4-dimethylthiazol-5-yl)-2-((1- | HepG2 | 0.1 | 501 |
| (pyrido[2,3-d]pyrimidin-4-yl)piperidin- | HEK-293 | 72.9 | |
| 4-yl)methyl)pyridazin-3(2H)-one | | | |

TABLE 6
IC₅₀ and therapeutic index of test compounds
using the ApoTox-Glo Triplex assay

| | Cell | IC₅₀ | Therapeutic |
|---|---|---|---|
| Compound name | line | (μM) | Index |
| 6-(2,4-dimethylthiazol-5-yl)-2-((1- | HepG2 | 0.1 | 396 |
| (pyrido[2,3-d]pyrimidin-4-yl)piperidin- | HEK-293 | 35.9 | |
| 4-yl)methyl)pyridazin-3(2H)-one | | | |

TABLE 7
Fold change of caspase 3/7 activation in HepG2 determined using ApoTox-Glo Triplex assay

| 6-(2,4-dimethylthiazol-5-yl)-2- | | | | | |
|---|---|---|---|---|---|
| ((1-(pyrido[2,3-d]pyrimidin-4- | | | | | |
| yl)piperidin-4- | | | | | |
| yl)methyl)pyridazin-3(2H)-one | | sAJM589 | | Taxol | |
| Compound | Fold-change of | Compound | Fold-change of | Compound | Fold-change of |
| concentration | caspase 3/7 | concentration | caspase 3/7 | concentration | caspase 3/7 |
| (nM) | activation | (nM) | activation | (nM) | activation |
| 10 | 1.245 | 100 | 1.215 | 0.1 | 1.149 |
| 31.6 | 1.204 | 1000 | 1.300 | 1 | 1.308 |
| 100 | 0.853 | 3160 | 1.127 | 3.16 | 1.436 |
| 316 | 0.586 | 10000 | 1.842 | 10 | 4.213 |
| 1000 | 0.420 | 40000 | 4.012 | 31.6 | 4.444 |
| 3160 | 0.371 | | | 100 | 5.021 |
| 10000 | 0.308 | | | 1000 | 4.920 |
| 20000 | 0.280 | | | | |

TABLE 8
Tumor volume over time following treatment with
vehicle and different doses of test compound

| | Tumor volume (mm³) | | | | | | | | | |
|---|---|---|---|---|---|---|---|---|---|---|
| | | | Test compound | | | | | | | |
| | Vehicle | | 15 mg/kg | | 35 mg/kg | | 50 mg/kg | | 100 mg/kg | |
| Day | Mean | SD | Mean | SD | Mean | SD | Mean | SD | Mean | SD |
| 0 | 93 | 63 | 93 | 54 | 92 | 33 | 94 | 45 | 88 | 36 |
| 2 | 140 | 82 | 137 | 62 | 129 | 70 | 123 | 50 | 126 | 56 |
| 5 | 217 | 115 | 212 | 66 | 191 | 75 | 191 | 67 | 186 | 51 |
| 8 | 348 | 169 | 330 | 80 | 307 | 100 | 295 | 78 | 290 | 74 |
| 10 | 467 | 200 | 435 | 133 | 390 | 113 | 368 | 88 | 363 | 98 |
| 12 | 583 | 218 | 534 | 127 | 485 | 135 | 463 | 95 | 458 | 126 |
| 14 | 808 | 279 | 749 | 141 | 739 | 113 | 716 | 124 | 652 | 144 |
| 16 | 1016 | 247 | 921 | 171 | 897 | 140 | 875 | 130 | 782 | 137 |
| 19 | 1267 | 174 | 1157 | 184 | 1101 | 118 | 1078 | 122 | 933 | 150 |
| 21 | 1623 | 154 | 1494 | 186 | 1459 | 111 | 1397 | 128 | 1158 | 114 |

TABLE 9
Concentrations of test compound in the plasma
and MYC biomarker levels in tumors following
treatment with different doses of test compound

| | Test compound | MYC protein |
|---|---|---|
| | concentrations | levels in tumor |
| | in plasma 8 h | homogenate |
| | after last | (ng/mg tumor |
| | dose (ng/ml) | homogenate) |
| Treatment | Mean ± SD | Mean ± SD |
| Vehicle control | Not determined | 41.93 ± 5.38 |
| 15 mg/kg test compound | 10.3 ± 1.7 | 35.70 ± 2.45 |
| 35 mg/kg test compound | 42.6 ± 29.1 | 29.55 ± 2.93 |
| 50 mg/kg test compound | 59.4 ± 21.9 | 29.11 ± 4.66 |
| 100 mg/kg test compound | 230.0 ± 111.0 | 23.53 ± 4.28 |

1. A compound of formula (I): 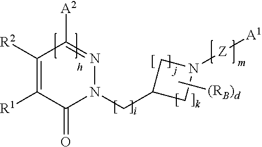
wherein
A¹ is hydrogen, C₁₋₇-alkyl, C₂₋₇-alkenyl, C₃₋₈-cycloalkyl, aryl, or heterocyclyl;
A² is hydrogen, C₁₋₇-alkyl, C₂₋₇-alkenyl, C₃₋₈-cycloalkyl, aryl, or heterocyclyl;
R¹ is hydrogen, halo, C₁₋₇-alkyl, C₂₋₇-alkenyl, C₃₋₈-cycloalkyl, aryl, or heterocyclyl;
R² is hydrogen, halo, C₁₋₇-alkyl, C₂₋₇-alkenyl, C₃₋₈-cycloalkyl, aryl, or heterocyclyl;
or A² and R² together form C₁₋₇-alkylene, or C₂₋₇-alkenylene;
Z is C₁₋₇-alkylene, C₂₋₇-alkenylene, —CH₂—, —(CH₂)₂—, —CH₂(CH)₂—, —C(═O)—, —C(═O)CH₂—, —C(═O)(CH₂)₂—, —C(═O)CH₂O—, —C(═O)(CH₂)₂O—, —C(═O)CH(CH₃)O—, —C(═O)O—, —C(═O)OCH₂—, —C(═O)O(CH₂)₂—, —C(═O)NH—, —C(═O)NHCH₂—, —C(═O)NH(CH₂)₂—, —S(═O)₂—, —S(═O)₂CH₂—, or —S(═O)₂(CH)₂—;
h is 1, 2, or 3;
i is 0, 1 or 2;
j is 0, 1, 2 or 3;
k is 0, 1, 2 or 3;
m is 0 or 1;
R_{B} represents non-hydrogen substituent(s), wherein each R_{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein the R_{B} substituent(s), if any, is covalently attached to any ring atom(s) in place of hydrogen, provided the maximum valency of the ring atom(s) to which R_{B} is attached is not exceeded;
d is 0, 1, 2, 3, 4, 5, 6, 7, 8, 9, 10, 11, 12, or 13;
wherein C₁₋₇-alkyl, C₂₋₇-alkenyl, C₁₋₇-alkylene, C₂₋₇-alkenylene, C₃₋₈-cycloalkyl, aryl, and heterocyclyl, are each independently substituted or unsubstituted;
with the proviso that j and k are not both 0 in the same compound;
and pharmaceutically acceptable salts, tautomers, N-oxides, and solvates thereof.

2. A compound of claim 1, wherein j is 2 and k is 2, which is represented by formula (II): 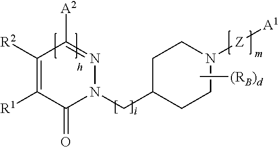
wherein
A¹ is hydrogen, C₁₋₇-alkyl, C₂₋₇-alkenyl, C₃₋₈-cycloalkyl, aryl, or heterocyclyl;
A² is hydrogen, C₁₋₇-alkyl, C₂₋₇-alkenyl, C₃₋₈-cycloalkyl, aryl, or heterocyclyl;
R¹ is hydrogen, halo, C₁₋₇-alkyl, C₂₋₇-alkenyl, C₃₋₈-cycloalkyl, aryl, or heterocyclyl;
R² is hydrogen, halo, C₁₋₇-alkyl, C₂₋₇-alkenyl, C₃₋₈-cycloalkyl, aryl, or heterocyclyl;
or A² and R² together form C₁₋₇-alkylene, or C₂₋₇-alkenylene;
Z is C₁₋₇-alkylene, C₂₋₇-alkenylene, —CH₂—, —(CH₂)₂—, —CH₂(CH)₂—, —C(═O)—, —C(═O)CH₂—, —C(═O)(CH₂)₂—, —C(═O)CH₂O—, —C(═O)(CH₂)₂O—, —C(═O)CH(CH₃)O—, —C(═O)O—, —C(═O)OCH₂—, —C(═O)O(CH₂)₂—, —C(═O)NH—, —C(═O)NHCH₂—, —C(═O)NH(CH₂)₂—, —S(═O)₂—, —S(═O)₂CH₂—, or —S(═O)₂(CH)₂—;
h is 1, 2, or 3;
i is 0, 1 or 2;
m is 0 or 1;
R_{B} represents non-hydrogen substituent(s), wherein each R_{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein the R_{B} substituent(s), if any, is covalently attached to any ring atom(s) in place of hydrogen, provided the maximum valency of the ring atom(s) to which R_{B} is attached is not exceeded;
d is 0, 1, 2, 3, 4, 5, 6, 7, 8, or 9;
wherein C₁₋₇-alkyl, C₂₋₇-alkenyl, C₁₋₇-alkylene, C₂₋₇-alkenylene, C₃₋₈-cycloalkyl, aryl, and heterocyclyl, are each independently substituted or unsubstituted;
and pharmaceutically acceptable salts, tautomers, N-oxides, and solvates thereof.

3. The compound of claim 1, wherein
A¹ is hydrogen, C₁₋₇-alkyl, C₂₋₇-alkenyl, C₃₋₈-cycloalkyl, aryl, or heterocyclyl;
A² is hydrogen, C₁₋₇-alkyl, C₂₋₇-alkenyl, C₃₋₈-cycloalkyl, aryl, or heterocyclyl;
R¹ is hydrogen or C₁₋₇-alkyl;
R² is hydrogen or C₁₋₇-alkyl;
his 1;
i is 1;
m is 0 and the Z moiety bridging A¹ and N in formula (I) or (II) is replaced by a direct single covalent bond between A¹ and N;
R_{B} is bromo, chloro, fluoro, or iodo;
wherein the R_{B} substituent(s), if any, is covalently attached to any ring atom(s) in place of hydrogen, provided the maximum valency of the ring atom(s) to which R_{B} is attached is not exceeded;
d is 0, 1, 2, 3, or 4;
wherein C₁₋₇-alkyl, C₂₋₇-alkenyl, C₁₋₇-alkylene, C₂₋₇-alkenylene, C₃₋₈-cycloalkyl, aryl, and heterocyclyl, are each independently substituted or unsubstituted;
and pharmaceutically acceptable salts, tautomers, N-oxides, and solvates thereof.

4. The compound of claim 2, wherein 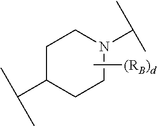
is selected from the group consisting of.

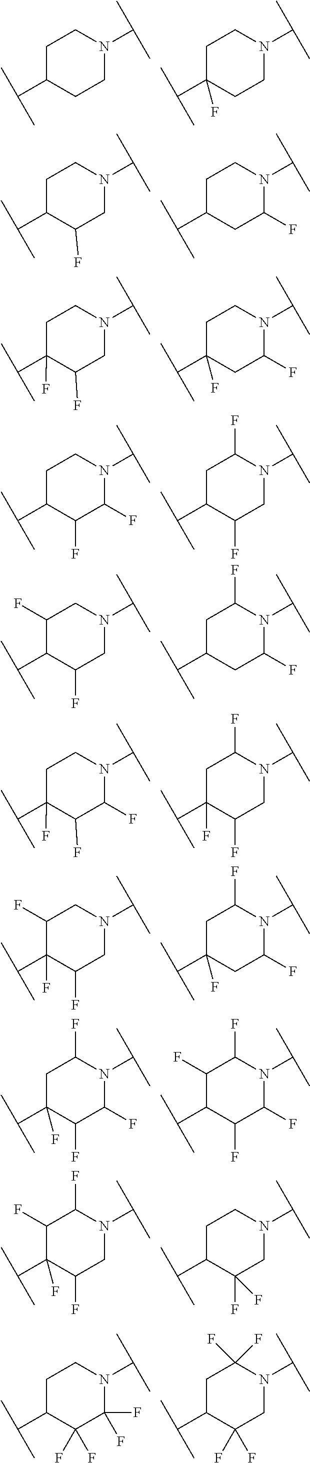

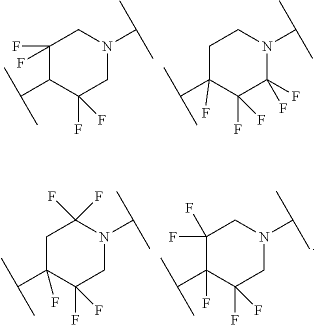

5. The compound of claim 1, wherein A¹ is selected from the group consisting of: 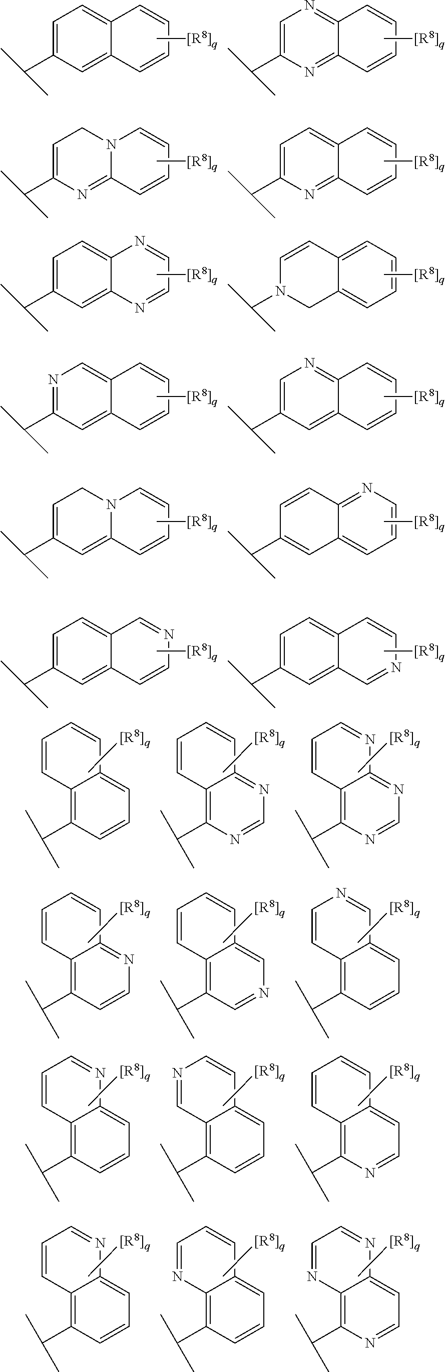 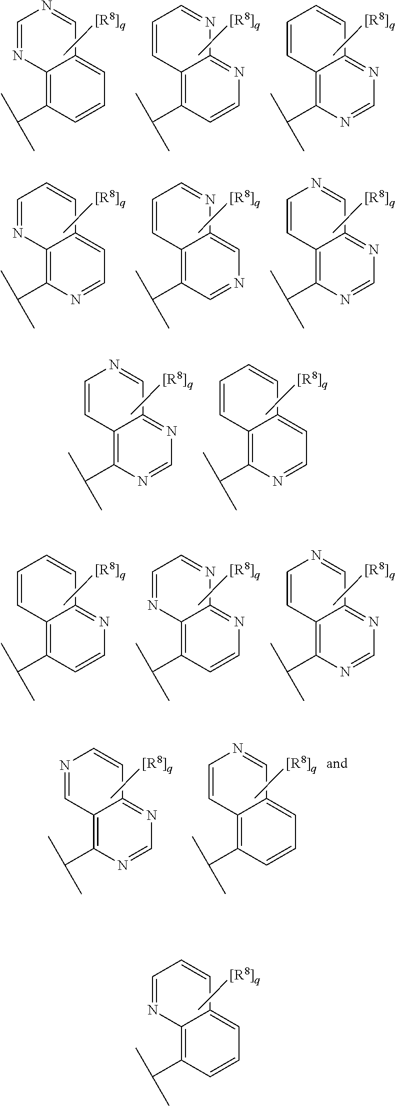
wherein
R_{B} represents non-hydrogen substituent(s), wherein each R_{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A¹ in place of hydrogen, including for either or both of the 6-membered rings, provided the maximum valency of the ring atom(s) to which R^{B} is attached is not exceeded;
q is 0, 1, 2, 3, 4, 5, 6, 7, or 8.

6. The compound of claim 5, wherein A¹ is selected from the group consisting of:

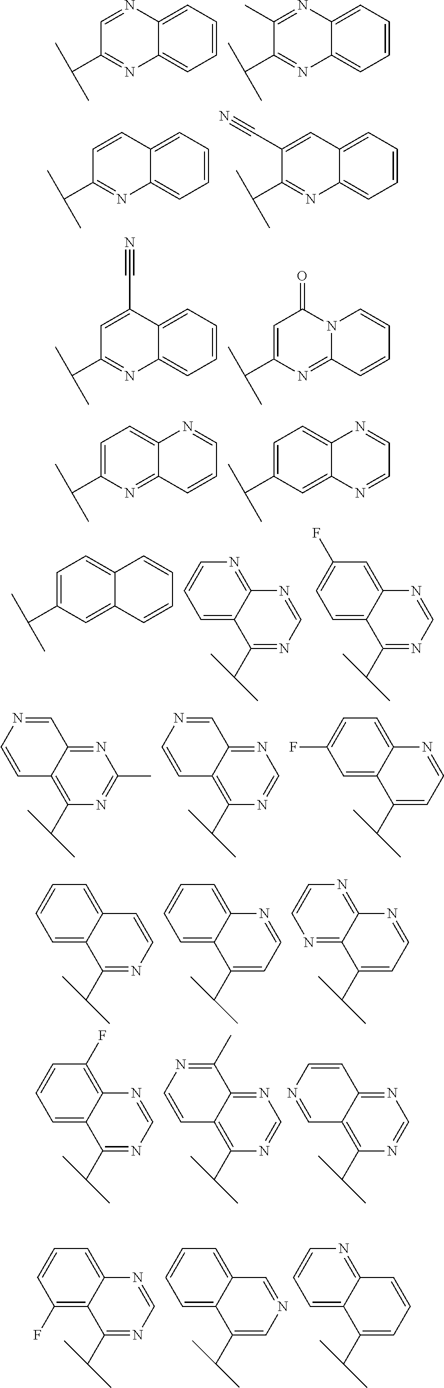

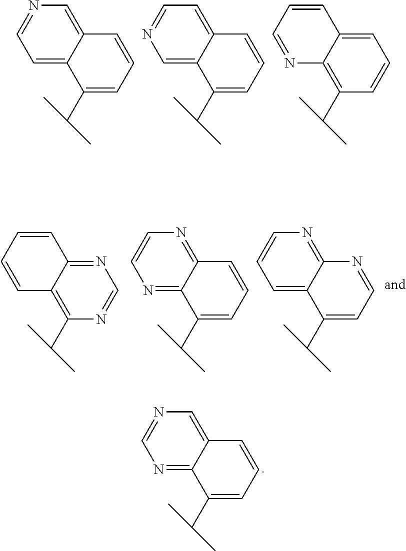

7. The compound of claim 1, wherein A² is selected from the group consisting of: 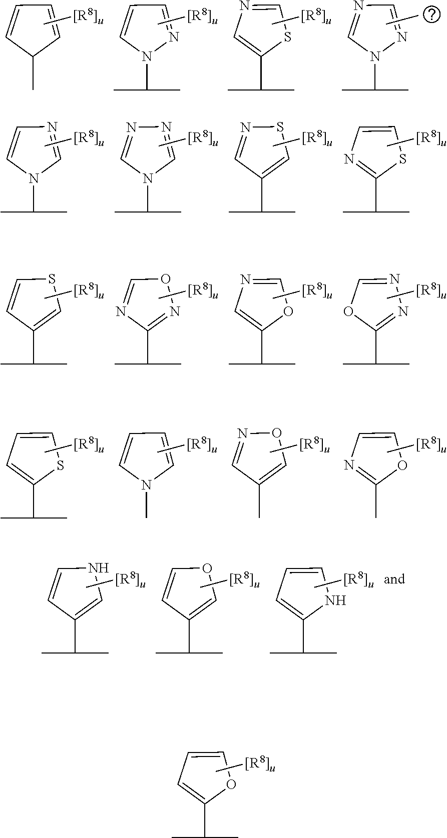
wherein
R^{B} represents non-hydrogen substituent(s), wherein each R^{B} is independently deuterium, C₁₋₇-alkyl (e.g., methyl, ethyl, propyl, isopropyl, etc.), C₃₋₈-cycloalkyl (e.g., cyclopropyl, cyclobutyl, etc.), halo (e.g., bromo, chloro, fluoro, or iodo), haloalkyl (e.g., monofluoromethyl, difluoromethyl, trifluoromethyl, etc.), cyano, methoxy, hydroxyl, formyl, acetyl, 2-hydroxyacetyl, 2-hydroxypropanal, formamidyl, sulfonyl, methylsulfonyl, ethylsulfonyl, 1-methylcarboxamido, N-ethylcarboxamido, or N,N-dimethylamino;
wherein one or more R^{B} substituent(s) can be covalently attached to any ring atom(s) of A² in place of hydrogen, provided the maximum valency of the ring atom(s) to which R^{B} is attached is not exceeded;
u is 0, 1, 2, 3, or 4.

8. The compound of claim 7, wherein A² is selected from the group consisting of:

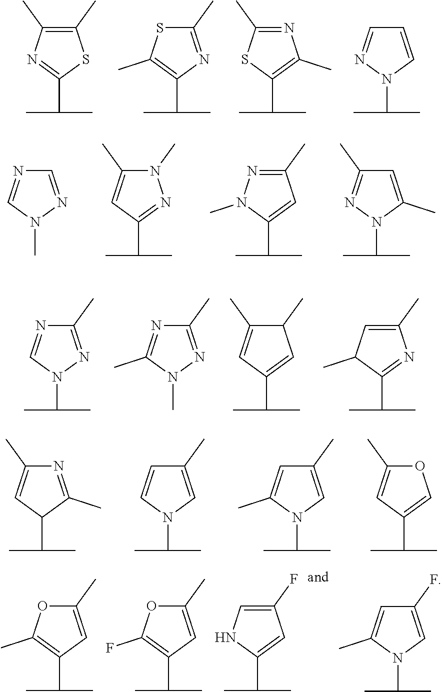

9. A compound selected from the group consisting of:
6-(2,4-dimethylthiazol-5-yl)-2-((1-(pyrido[2,3-d]pyrimidin-4-yl)piperidin-4-yl)methyl)pyridazin-3(2H)-one;
2-((1-(7-fluoroquinazolin-4-yl)piperidin-4-yl)methyl)-6-(1H-pyrazol-1-yl)pyridazin-3(2H)-one;
6-(2,4-dimethylthiazol-5-yl)-2-((1-(6-methylpyrimidin-4-yl)piperidin-4-yl)methyl)pyridazin-3(2H)-one;
6-(2,4-dimethylthiazol-5-yl)-2-((1-(2-methylpyrido[3,4-d]pyrimidin-4-yl)piperidin-4-yl)methyl)pyridazin-3(2H)-one;
2-((1-(9H-purin-6-yl)piperidin-4-yl)methyl)-6-(2,4-dimethylthiazol-5-yl)pyridazin-3(2H)-one;
2-((1-(6-fluoroquinazolin-4-yl)piperidin-4-yl)methyl)-6-(1H-1,2,4-triazol-1-yl)pyridazin-3(2H)-one;
2-((1-(1,6-dimethyl-1H-pyrazolo[3,4-d]pyrimidin-4-yl)azetidin-3-yl)methyl)-6-(pyridin-4-yl)pyridazin-3(2H)-one;
2-(1-(1,6-dimethyl-1H-pyrazolo[3,4-d]pyrimidin-4-yl)azetidin-3-yl)-6-(pyridin-4-yl)pyridazin-3(2H)-one;
6-(1H-pyrazol-1-yl)-2-((1-(3-(trifluoromethyl)-[1,2,4]triazolo[4,3-b]pyridazin-6-yl)piperidin-4-yl)methyl)pyridazin-3(2H)-one;
6-(1H-1,2,4-triazol-1-yl)-2-((1-(3-(trifluoromethyl)-[1,2,4]triazolo[4,3-b]pyridazin-6-yl)piperidin-4-yl)methyl)pyridazin-3(2H)-one;
2-((1-(9H-purin-6-yl)azetidin-3-yl)methyl)-6-(pyridin-4-yl)pyridazin-3(2H)-one;
2-((1-(6-methylpyrazin-2-yl)piperidin-4-yl)methyl)-6-(1H-1,2,4-triazol-1-yl)pyridazin-3(2H)-one;
6-(3,5-dimethyl-1H-pyrazol-1-yl)-2-((1-(3-methyl-[1,2,4]triazolo[4,3-b]pyridazin-6-yl)azetidin-3-yl)methyl)pyridazin-3(2H)-one;
6-(3,5-dimethyl-1H-pyrazol-1-yl)-2-(1-(3-methyl-3H-imidazo[4,5-b]pyridin-2-yl)piperidin-4-yl)pyridazin-3(2H)-one;
6-(2,4-dimethyl-1,3-thiazol-5-yl)-2-(3-fluoro-1-pyrido[2,3-d]pyrimidin-4-ylpiperidin-4-yl)methylpyridazin-3-one;
6-(2,4-dimethyl-1,3-thiazol-5-yl)-2-(3,3-difluoro-1-pyrido[2,3-d]pyrimidin-4-ylpiperidin-4-yl)methylpyridazin-3-one;
6-(2,4-dimethyl-1,3-thiazol-5-yl)-2-(2-fluoro-1-pyrido[2,3-d]pyrimidin-4-ylpiperidin-4-yl)methylpyridazin-3-one;
6-(2,4-dimethyl-1,3-thiazol-5-yl)-2-(2,2-difluoro-1-pyrido[2,3-d]pyrimidin-4-ylpiperidin-4-yl)methylpyridazin-3-one;
6-(2,4-dimethyl-1,3-thiazol-5-yl)-2-(2,3-difluoro-1-pyrido[2,3-d]pyrimidin-4-ylpiperidin-4-yl)methylpyridazin-3-one;
6-(2,4-dimethyl-1,3-thiazol-5-yl)-2-(2,4-difluoro-1-pyrido[2,3-d]pyrimidin-4-ylpiperidin-4-yl)methylpyridazin-3-one;
6-(2,4-dimethyl-1,3-thiazol-5-yl)-2-(2,5-difluoro-1-pyrido[2,3-d]pyrimidin-4-ylpiperidin-4-yl)methylpyridazin-3-one;
6-(2,4-dimethyl-1,3-thiazol-5-yl)-2-(2,6-difluoro-1-pyrido[2,3-d]pyrimidin-4-ylpiperidin-4-yl)methylpyridazin-3-one;
6-(2,4-dimethyl-1,3-thiazol-5-yl)-2-(3,4-difluoro-1-pyrido[2,3-d]pyrimidin-4-ylpiperidin-4-yl)methylpyridazin-3-one;
6-(2,4-dimethyl-1,3-thiazol-5-yl)-2-(3,5-difluoro-1-pyrido[2,3-d]pyrimidin-4-ylpiperidin-4-yl)methylpyridazin-3-one;
2-((1-(5H-pyrazolo[3,4-d]pyrimidin-4-yl)piperidin-4-yl)methyl)-6-(2,4-dimethylthiazol-5-yl)pyridazin-3(2H)-one;
6-(2,4-dimethyl-1,3-thiazol-5-yl)-2-[[1-(7H-purin-6-yl)piperidin-4-yl]methyl]pyridazin-3-one;
6-(2,4-dimethyl-1,3-thiazol-5-yl)-2-[[1-(6-methylpyrimidin-4-yl)piperidin-4-yl]methyl]pyridazin-3-one;
6-(2,4-dimethyl-1,3-thiazol-5-yl)-2-[[1-(2-methylpyrido[3,4-d]pyrimidin-4-yl)piperidin-4-yl]methyl]pyridazin-3-one;
6-(2,4-dimethyl-1,3-thiazol-5-yl)-2-[(1-pyrazolo[1,5-a]pyrimidin-5-ylpiperidin-4-yl)methyl]pyridazin-3-one;
6-(2,4-dimethyl-1,3-thiazol-5-yl)-2-[[1-(2-methylpyrimidin-4-yl)piperidin-4-yl]methyl]pyridazin-3-one;
6-(2,4-dimethyl-1,3-thiazol-5-yl)-2-[[1-(2-propan-2-ylpyrimidin-4-yl)piperidin-4-yl]methyl]pyridazin-3-one;
6-(2,4-dimethyl-1,3-thiazol-5-yl)-2-[[1-(9-methylpurin-6-yl)piperidin-4-yl]methyl]pyridazin-3-one;
6-(2,4-dimethylthiazol-5-yl)-2-((1-(6-methyl-3H-pyrrolo[3,2-d]pyrimidin-4-yl)piperidin-4-yl)methyl)pyridazin-3(2H)-one;
6-(2,4-dimethyl-1,3-thiazol-5-yl)-2-[[1-(6-ethyl-5-fluoropyrimidin-4-yl)piperidin-4-yl]methyl]pyridazin-3-one;
6-(2,4-dimethyl-1,3-thiazol-5-yl)-2-[(1-thieno[2,3-d]pyrimidin-4-ylpiperidin-4-yl)methyl]pyridazin-3-one;
6-(2,4-dimethylthiazol-5-yl)-2-((1-(imidazo[1,2-b]pyridazin-6-yl)piperidin-4-yl)methyl)pyridazin-3(2H)-one;
2-[4-[[3-(2,4-dimethyl-1,3-thiazol-5-yl)-6-oxopyridazin-1-yl]methyl]piperidin-1-yl]pyridine-4-carbonitrile;
6-[4-[[3-(2,4-dimethyl-1,3-thiazol-5-yl)-6-oxopyridazin-1-yl]methyl]piperidin-1-yl]pyridine-3-carbonitrile;
6-(2,4-dimethyl-1,3-thiazol-5-yl)-2-[(1-thieno[3,2-d]pyrimidin-4-ylpiperidin-4-yl)methyl]pyridazin-3-one;
2-[4-[[3-(2,4-dimethyl-1,3-thiazol-5-yl)-6-oxopyridazin-1-yl]methyl]piperidin-1-yl]pyridine-3-carbonitrile;
6-(2,4-dimethylthiazol-5-yl)-2-((1-(6-(trifluoromethyl)pyridin-2-yl)piperidin-4-yl)methyl)pyridazin-3(2H)-one;
6-(2,4-dimethylthiazol-5-yl)-2-((1-(4-(trifluoromethyl)pyridin-2-yl)piperidin-4-yl)methyl)pyridazin-3(2H)-one;
6-(2,4-dimethyl-1,3-thiazol-5-yl)-2-[[1-(5-methylpyrimidin-2-yl)piperidin-4-yl]methyl]pyridazin-3-one;
6-(2,4-dimethyl-1,3-thiazol-5-yl)-2-[(1-pyrimidin-2-ylpiperidin-4-yl)methyl]pyridazin-3-one;
6-(2,4-dimethylthiazol-5-yl)-2-((1-(5-(trifluoromethyl)pyridin-2-yl)piperidin-4-yl)methyl)pyridazin-3(2H)-one;
6-(2,4-dimethyl-1,3-thiazol-5-yl)-2-[[1-(3-methyl-[1,2,4]triazolo[4,3-b]pyridazin-6-yl)piperidin-4-yl]methyl]pyridazin-3-one;
6-(2,4-dimethyl-1,3-thiazol-5-yl)-2-[(1-pyrazolo[1,5-a]pyrazin-4-ylpiperidin-4-yl)methyl]pyridazin-3-one;
6-(2,4-dimethylthiazol-5-yl)-2-((1-(3-(trifluoromethyl)pyridin-2-yl)piperidin-4-yl)methyl)pyridazin-3(2H)-one;
2-[[1-(5-chloropyrimidin-2-yl)piperidin-4-yl]methyl]-6-(2,4-dimethyl-1,3-thiazol-5-yl)pyridazin-3-one;
6-(2,4-dimethyl-1,3-thiazol-5-yl)-2-[[1-(4-methylpyrimidin-2-yl)piperidin-4-yl]methyl]pyridazin-3-one;
6-(2,4-dimethyl-1,3-thiazol-5-yl)-2-[(1-pyrido[2,3-d]pyrimidin-4-ylpiperidin-4-yl)methyl]pyridazin-3-one;
2-[[1-(4,6-dimethylpyrimidin-2-yl)piperidin-4-yl]methyl]-6-(2,4-dimethyl-1,3-thiazol-5-yl)pyridazin-3-one;
6-(2,4-dimethyl-1,3-thiazol-5-yl)-2-[[1-[(5-methyl-1,2-oxazol-3-yl)methyl]piperidin-4-yl]methyl]pyridazin-3-one;
6-(2,4-dimethylthiazol-5-yl)-2-((1-(5,6,7,8-tetrahydroquinazolin-4-yl)piperidin-4-yl)methyl)pyridazin-3(2H)-one;
6-(2,4-dimethyl-1,3-thiazol-5-yl)-2-[(1-quinoxalin-2-ylpiperidin-4-yl)methyl]pyridazin-3-one;
6-(2,4-dimethyl-1,3-thiazol-5-yl)-2-[[1-(5-fluoropyrimidin-2-yl)piperidin-4-yl]methyl]pyridazin-3-one;
5-[4-[[3-(2,4-dimethyl-1,3-thiazol-5-yl)-6-oxopyridazin-1-yl]methyl]piperidin-1-yl]pyridine-2-carbonitrile;
6-(2,4-dimethyl-1,3-thiazol-5-yl)-2-[[1-(2-methylpyrazolo[1,5-a]pyrazin-4-yl)piperidin-4-yl]methyl]pyridazin-3-one;
2-[4-[[3-(2,4-dimethyl-1,3-thiazol-5-yl)-6-oxopyridazin-1-yl]methyl]piperidin-1-yl]pyrimidine-4-carbonitrile;
6-(2,4-dimethyl-1,3-thiazol-5-yl)-2-[[1-(4-methoxypyrimidin-2-yl)piperidin-4-yl]methyl]pyridazin-3-one;
6-(2,4-dimethyl-1,3-thiazol-5-yl)-2-(piperidin-4-ylmethyl)pyridazin-3-one;
N-[2-(7-cyclohexyl-6-imino-13-methyl-2-oxo-1,7,9-triazatricyclo[8.4.0.03,8]tetradeca-3(8),4,9,11,13-pentaen-5-yl)-4-phenyl-1,3-thiazol-5-yl]-4-methoxybenzamide;
3-(4-((3-(2,4-dimethylthiazol-5-yl)-6-oxopyridazin-1(6H)-yl)methyl)piperidin-1-yl)pyrazine-2-carbonitrile;
4-[[4-[[3-(2,4-dimethyl-1,3-thiazol-5-yl)-6-oxopyridazin-1-yl]methyl]piperidin-1-yl]methyl]benzonitrile;
6-(2,4-dimethylthiazol-5-yl)-2-((1-(2-fluorobenzyl)piperidin-4-yl)methyl)pyridazin-3(2H)-one;
6-(2,4-dimethylthiazol-5-yl)-2-((1-(4-fluorobenzyl)piperidin-4-yl)methyl)pyridazin-3(2H)-one;
6-(2,4-dimethylthiazol-5-yl)-2-((1-(3-fluorobenzyl)piperidin-4-yl)methyl)pyridazin-3(2H)-one;
2-((1-(2-chlorobenzyl)piperidin-4-yl)methyl)-6-(2,4-dimethylthiazol-5-yl)pyridazin-3(2H)-one;
2-((1-(4-chlorobenzyl)piperidin-4-yl)methyl)-6-(2,4-dimethylthiazol-5-yl)pyridazin-3(2H)-one;
2-((1-(3-chlorobenzyl)piperidin-4-yl)methyl)-6-(2,4-dimethylthiazol-5-yl)pyridazin-3(2H)-one;
6-(2,4-dimethyl-1,3-thiazol-5-yl)-2-[[1-(3-methylquinoxalin-2-yl)piperidin-4-yl]methyl]pyridazin-3-one;
6-(2,4-dimethyl-1,3-thiazol-5-yl)-2-[[1-(2-methyl-6,7-dihydro-5H-cyclopenta[d]pyrimidin-4-yl)piperidin-4-yl]methyl]pyridazin-3-one;
2-[[1-[(2,5-difluorophenyl)methyl]piperidin-4-yl]methyl]-6-(2,4-dimethyl-1,3-thiazol-5-yl)pyridazin-3-one;
2-[[1-[(2,4-difluorophenyl)methyl]piperidin-4-yl]methyl]-6-(2,4-dimethyl-1,3-thiazol-5-yl)pyridazin-3-one;
2-[[1-[(3,4-difluorophenyl)methyl]piperidin-4-yl]methyl]-6-(2,4-dimethyl-1,3-thiazol-5-yl)pyridazin-3-one;
2-((1-(3,5-difluorobenzyl)piperidin-4-yl)methyl)-6-(2,4-dimethylthiazol-5-yl)pyridazin-3(2H)-one;
2-[[1-[(2-chloro-6-fluorophenyl)methyl]piperidin-4-yl]methyl]-6-(2,4-dimethyl-1,3-thiazol-5-yl)pyridazin-3-one;
2-[[1-(1,3-benzoxazol-2-yl)piperidin-4-yl]methyl]-6-(2,4-dimethyl-1,3-thiazol-5-yl)pyridazin-3-one;
2-(3-cyclopropyl-6-oxopyridazin-1-yl)-N-[2-[3-(2,4-dimethyl-1,3-thiazol-5-yl)-6-oxopyridazin-1-yl]ethyl]acetamide;
tert-butyl 4-[[3-(2,4-dimethyl-1,3-thiazol-5-yl)-6-oxopyridazin-1-yl]methyl]piperidine-1-carboxylate;
6-(2,4-dimethylthiazol-5-yl)-2-((1-(6-fluorobenzo[d]oxazol-2-yl)piperidin-4-yl)methyl)pyridazin-3(2H)-one;
N-[2-[3-(2,4-dimethyl-1,3-thiazol-5-yl)-6-oxopyridazin-1-yl]ethyl]-2-(4-oxoquinazolin-3-yl)acetamide;
N-[2-[3-(2,4-dimethyl-1,3-thiazol-5-yl)-6-oxopyridazin-1-yl]ethyl]-2-(1-methyltetrazol-5-yl)sulfanylacetamide;
N-[2-[3-(2,4-dimethyl-1,3-thiazol-5-yl)-6-oxopyridazin-1-yl]ethyl]thiophene-2-sulfonamide;
N-[2-[3-(2,4-dimethyl-1,3-thiazol-5-yl)-6-oxopyridazin-1-yl]ethyl]-2,3-dihydro-1,4-benzodioxine-6-sulfonamide;
N-[2-[3-(2,4-dimethyl-1,3-thiazol-5-yl)-6-oxopyridazin-1-yl]ethyl]-3,5-dimethyl-1,2-oxazole-4-sulfonamide;
2-(benzimidazol-1-yl)-N-[2-[3-(2,4-dimethyl-1,3-thiazol-5-yl)-6-oxopyridazin-1-yl]ethyl]acetamide;
N-[2-[3-(2,4-dimethyl-1,3-thiazol-5-yl)-6-oxopyridazin-1-yl]ethyl]-1-methylsulfonylpiperidine-4-carboxamide;
3-(3,5-dimethyl-1,2-oxazol-4-yl)-N-[2-[3-(2,4-dimethyl-1,3-thiazol-5-yl)-6-oxopyridazin-1-yl]ethyl]propanamide;
2-(1,2-benzoxazol-3-yl)-N-[2-[3-(2,4-dimethyl-1,3-thiazol-5-yl)-6-oxopyridazin-1-yl]ethyl]acetamide;
2-(1,3-benzothiazol-2-ylsulfanyl)-N-[2-[3-(2,4-dimethyl-1,3-thiazol-5-yl)-6-oxopyridazin-1-yl]ethyl]acetamide;
N-[2-[3-(2,4-dimethyl-1,3-thiazol-5-yl)-6-oxopyridazin-1-yl]ethyl]-4-methylthiadiazole-5-carboxamide;
N-[2-[3-(2,4-dimethyl-1,3-thiazol-5-yl)-6-oxopyridazin-1-yl]ethyl]-1-thiophen-2-ylcyclopentane-1-carboxamide;
3-(benzenesulfonyl)-N-[2-[3-(2,4-dimethyl-1,3-thiazol-5-yl)-6-oxopyridazin-1-yl]ethyl]propanamide;
1-[2-[3-(2,4-dimethyl-1,3-thiazol-5-yl)-6-oxopyridazin-1-yl]ethyl]-3-(naphthalen-1-ylmethyl)urea;
1-[2-[3-(2,4-dimethyl-1,3-thiazol-5-yl)-6-oxopyridazin-1-yl]ethyl]-3-(2-methoxyphenyl)urea;
N-[2-[3-(2,4-dimethyl-1,3-thiazol-5-yl)-6-oxopyridazin-1-yl]ethyl]furan-2-carboxamide;
or a pharmaceutically acceptable salt thereof.

10. The compound of claim 1, wherein one or more hydrogens is replaced with deuterium.

11. A pharmaceutical composition comprising the compound of claim 1, or a pharmaceutically acceptable salt thereof, and one or more pharmaceutically acceptable carriers, excipients, or diluents.

12. A method of reducing the expression or activity of MYC in cells or tissues comprising administering the compound of claim 1, to a cell, animal, or human such that expression or activity of MYC is reduced.

13. A method to treat, prevent or ameliorate cancer in a subject, comprising administering the compound of claim 1 to the subject.

14. The method of claim 13, wherein the cancer is a MYC-driven cancer.+

15-20. (canceled)

21. The compound of claim 9, wherein one or more hydrogens is replaced with deuterium.

22. A pharmaceutical composition comprising the compound of claim 9, or a pharmaceutically acceptable salt thereof, and one or more pharmaceutically acceptable carriers, excipients, or diluents.

23. A method of reducing the expression or activity of MYC in cells or tissues comprising administering the compound of claim 9 to a cell, animal, or human such that expression or activity of MYC is reduced.

24. A method to treat, prevent or ameliorate cancer in a subject, comprising administering the compound of claim 9 to the subject.

25. The method of claim 24, wherein the cancer is a MYC-driven cancer.


SEQUENCE LISTING (7 sequences)

SEQ ID NO: 1 - DNA, 7518 bp, Homo sapiens
  source 1..7518: mol_type = genomic DNA
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccaggtaagcaccgaagtccacttgccttttaatttatttttttatcactttaatgctgagatgagtcgaatgcctaaatagggtgtcttttctcccattcctgcgctattgacacttttctcagagtagttatggtaactggggctggggtggggggtaatccagaactggatcggggtaaagtgacttgtcaagatgggagaggagaaggcagagggaaaacgggaatggtttttaagactaccctttcgagatttctgccttatgaatatattcacgctgactcccggccggtcggacattcctgctttattgtgttaattgctctctgggttttggggggctgggggttgctttgcggtgggcagaaagccccttgcatcctgagctccttggagtagggaccgcatatcgcctgtgtgagccagatcgctccgcagccgctgacttgtccccgtctccgggagggcatttaaatttcggctcaccgcatttctgacagccggagacggacactgcggcgcgtcccgcccgcctgtccccgcggcgattccaacccgccctgatccttttaagaagttggcatttggctttttaaaaagcaataatacaatttaaaacctgggtctctagaggtgttaggacgtggtgttgggtaggcgcaggcaggggaaaagggaggcgaggatgtgtccgattctcctggaatcgttgacttggaaaaaccagggcgaatctccgcacccagccctgactcccctgccgcggccgccctcgggtgtcctcgcgcccgagatgcggaggaactgcgaggagcggggctctgggcggttccagaacagctgctacccttggtggggtggctccgggggaggtatcgcagcggggtctctggcgcagttgcatctccgtattgagtgcgaagggaggtgcccctattattatttgacaccccccttgtatttatggaggggtgttaaagcccgcggctgagctcgccactccagccggcgagagaaagaagaaaagctggcaaaaggagtgttggacgggggcggtactgggggtggggacgggggcggtggagagggaaggttgggaggggctgcggtgccggcgggggtaggagagcggctagggcgcgagtgggaacagccgcagcggaggggccccggcgcggagcggggttcacgcagccgctagcgcccaggcgcctctcgccttctccttcaggtggcgcaaaactttgtgccttggattttggcaaattgttttcctcaccgccacctcccgcggcttcttaagggcgccagggccgatttcgattcctctgccgctgcggggccgactcccgggctttgcgctccgggctcccgggggagcgggggctcggcgggcaccaagccgctggttcactaagtgcgtctccgagatagcaggggactgtccaaagggggtgaaagggtgctccctttattcccccaccaagaccacccagccgctttaggggatagctctgcaaggggagaggttcgggactgtggcgcgcactgcgcgctgcgccaggtttccgcaccaagacccctttaactcaagactgcctcccgctttgtgtgccccgctccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctggtaagcgaagcccgcccaggcctgtcaaaagtgggcggctggatacctttcccattttcattggcagcttatttaacgggccactcttattaggaaggagagatagcagatctggagagatttgggagctcatcacctctgaaaccttgggctttagcgtttcctcccatcccttccccttagactgcccatgtttgcagcccccctccccgtttgtctcccacccctcaggaatttcatttaggtttttaaaccttctggcttatcttacaactcaatccacttcttcttacctcccgttaacattttaattgccctggggcggggtggcagggagtgtatgaatgaggataagagaggattgatctctgagagtgaatgaattgcttccctcttaacttccgagaagtggtgggatttaatgaactatctacaaaaatgaggggctgtgtttagaggctaggcagggcctgcctgagtgcgggagccagtgaactgcctcaagagtgggtgggctgaggagctgggatcttctcagcctattttgaacactgaaaagcaaatccttgccaaagttggacttttttttttcttttattccttcccccgccctcttggacttttggcaaaactgcaatttttttttttttatttttcatttccagtaaaatagggagttgctaaagtcataccaagcaatttgcagctatcatttgcaacacctgaagtgttcttggtaaagtccctcaaaaataggaggtgcttgggaatgtgctttgctttgggtgtgtccaaagcctcattaagtcttaggtaagaattggcatcaatgtcctatcctgggaagttgcacttttcttgtccatgccataacccagctgtctttccctttatgagactcttaccttcatggtgagaggagtaagggtggctggctagattggttctttttttttttttttccttttttaagacggagtctcactctgtcactaggctggagtgcagtggcgcaatcaacctccaaccccctggttcaagagattctcctgcctcagcctcccaagtagctgggactacaggtgcacaccaccatgccaggctaatttttgtaattttagtagagatggggtttcatcgtgttggccaggatggtctctcctgacctcacgatccgcccacctcggcctcccaaagtgctgggattacaggtgtgagccagggcaccaggcttagatgtggctctttggggagataattttgtccagagacctttctaacgtattcatgccttgtatttgtacagcattaatctggtaattgattattttaatgtaaccttgctaaaggagtgatttctatttcctttcttaaagaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 2 - RNA, 3721 nt, Homo sapiens
  source 1..3721: mol_type = mRNA
aactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 3 - RNA, 4515 nt, Homo sapiens
  source 1..4515: mol_type = mRNA
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 4 - DNA, 20 bp, synthetic construct
  source 1..20: mol_type = other DNA; note = Synthetic oligonucleotide
accaccagcagcgactctga

SEQ ID NO: 5 - DNA, 23 bp, synthetic construct
  source 1..23: mol_type = other DNA; note = Synthetic oligonucleotide
tccagcagaaggtgatccagact

SEQ ID NO: 6 - DNA, 21 bp, synthetic construct
  source 1..21: mol_type = other DNA; note = Synthetic oligonucleotide
aggcatcctcaccctgaagta

SEQ ID NO: 7 - DNA, 20 bp, synthetic construct
  source 1..20: mol_type = other DNA; note = Synthetic oligonucleotide
cacacgcagctcattgtaga